(19) United States
(12) Patent Application Publication
(10) Pub. No.: US 2026/0317268 A1
(43) Pub. Date: Oct. 15, 2026

(54) NOVEL IONIZABLE LIPID DERIVATIZED AT ORTHO POSITION AND CONTAINING AROMATIC ESTER

(71) Applicants: NOF CORPORATION, Tokyo (JP); TOHOKU UNIVERSITY, Sendai-shi (JP)

(72) Inventors: Ryosuke MOTOUSU, Kawasaki-shi (JP); Tokihiro TANAKA, Kawasaki-shi (JP); Masato FUJII, Yokohama-shi (JP); Yuzuka TOMITA, Kawasaki-shi (JP); Hidetaka AKITA, Sendai-shi (JP); Hiroki TANAKA, Sendai-shi (JP)

(73) Assignees: NOF CORPORATION, Tokyo (JP); TOHOKU UNIVERSITY, Sendai-shi (JP)

(21) Appl. No.: 19/577,472

(22) Filed: Mar. 25, 2026

(30) Foreign Application Priority Data
Mar. 27, 2025 (JP) 2025-053856

(51) Int. Cl. C07C 233/38; A61K 9/1272; A61K 47/18; A61K 47/22; A61K 48/00; C07C 219/28; C07C 233/36; C07C 323/41; C07D 207/09; C07D 207/14; C07D 209/44; C07D 211/14; C07D 211/58; C07D 213/40; C07D 215/22; C07D 249/08; C07D 261/08; C07D 277/28; C07D 295/096; C07D 295/13; C07D 295/26; C07D 307/38; C07D 333/20; C07D 401/04; C07D 401/06; C07D 401/12; C07D 405/06; C07D 413/06; C07D 417/12; C07D 453/02; C07D 455/04; C07D 495/04

(52) U.S. Cl. CPC C07C 233/38; A61K 9/1272; A61K 47/18; A61K 47/22; A61K 48/0033; A61K 48/005; C07C 219/28; C07C 233/36; C07C 323/41; C07D 207/09; C07D 207/14; C07D 209/44; C07D 211/14; C07D 211/58; C07D 213/40; C07D 215/22; C07D 249/08; C07D 261/08; C07D 277/28; C07D 295/096; C07D 295/13; C07D 295/26; C07D 307/38; C07D 333/20; C07D 401/04; C07D 401/06; C07D 401/12; C07D 405/06; C07D 413/06; C07D 417/12; C07D 453/02; C07D 455/04; C07D 495/04; C07C 2601/14

(57) ABSTRACT

An ionizable lipid that contains an ester moiety having high degradability and can be used as a nucleic acid delivery carrier having good intracellular pharmacokinetics is provided. The ionizable lipid used for delivering pharmaceuticals, contains at least one linear or cylcic tertiary amino group, at least one phenyl group having an ester bond at an o-position, and an aliphatic hydrocarbon group having 4 or more carbon atoms.

### FIELD OF THE INVENTION

The present invention relates to an ionizable lipid, a lipid membrane structure, a nucleic acid transfer agent, and a pharmaceutical composition containing the ionizable lipid, a method for transferring a nucleic acid into cells or target cells, and a method for producing a cellular medicine.

### BACKGROUND OF THE INVENTION

An effective and safe nucleic acid delivery carrier is being demanded for practically realizing nucleic acid therapeutics. While viral vectors are nucleic acid delivery carriers having a good nucleic acid delivery efficiency, non-viral nucleic acid delivery carriers capable of being used more safely are being developed, and among them, a carrier using an ionizable lipid, such as a ionizable cationic lipid, is a non-viral nucleic acid delivery carrier that is most commonly used at present.

The ionizable cationic lipid is broadly constituted by an amine moiety and a lipid moiety. The ionizable cationic lipid promotes cellular uptake of a nucleic acid and delivers the nucleic acid to the cells by forming a liposome or a lipid membrane structure through electrostatic interaction between the amine moiety showing cationicity and the nucleic acid as a polyanion.

Examples of the known ionizable cationic lipids having been widely used include 1,2-dioleoyloxy-3-trimethylammoniumpropane (DOTAP) and 1,2-dioleoyloxy-3-dimethylaminopropane (DODAP). These known ionizable cationic lipids each are combined with a phospholipid to form a liposome or a lipid membrane structure that is positively charged, which is allowed to interact electrostatically with a nucleic acid, so as to deliver the nucleic acid to the target cells (see, for example, NPL 1).

On the other hand, in order that the lipid membrane structure using a ionizable cationic lipid exerts the practical effect in vivo as the nucleic acid delivery carrier, the lipid membrane structure necessarily shows the particular systemic pharmacokinetics. Specifically, the lipid membrane structure necessarily satisfies such requirements as high stability in the blood. In order to address the problem, it has been known that the lipid membrane structure that has a pKa of the surface of the lipid membrane structure that is regulated to around neutral and has a PEG lipid introduced thereto shows a long lifetime in the blood after intravenous injection, and accumulates at a tumor site. Furthermore, there is an example in which the systemic pharmacokinetics of the lipid membrane structure are improved by regulating the surface pKa thereof.

For example, NPL 2 and NPL 3 show that the systemic pharmacokinetics and the distribution in cells in the liver of the lipid membrane structure can be controlled by regulating the surface pKa thereof. These literatures show that for the endosomal escape, the escape of the lipid membrane structure from endosomes is promoted by regulating the surface pKa of the lipid membrane structure, and a nucleic acid can be efficiently delivered into the cytoplasm.

### CITATION LIST

### Non-Patent Literatures

NPL 1: Biomaterials, 29 (24-25): 3477-96, 2008
NPL 2: Molecular Therapy, 24 (4): 788-795, 2016
NPL 3: Angewandte Chemie International Edition, 51:8529-8533, 2012
NPL 4: Molecular Therapy, 13 (4): 786-794, 2006
NPL 5: Adv. Funct. Mater., 30, 1910575, 2020
NPL 6: Biomater. Sci., 2024, 12, 3027-3044

### SUMMARY OF THE INVENTION

### Technical Problem

While the ionizable cationic lipids improved in systemic pharmacokinetics have been developed as described above, in general, the ionizable cationic lipids are demanded to exert a larger effect with a smaller uptake amount thereof in consideration of the nature of the nucleic acid delivery carrier, i.e., the transfer of foreign substances into the cells. Specifically, in the case where the lipid membrane structure is used as a delivery carrier of expression vectors into the cells, it is demanded that the expression level per unit lipid membrane structure taken up into the cells is increased to enhance the expression efficiency in the cells. More specifically, in order to enhance the expression efficiency in the cells, it is necessary to improve the intracellular pharmacokinetics, such as the uptake to the cells, the escape from endosomes, and the transfer through the nuclear membrane, in addition to the systemic pharmacokinetics (see, for example, NPL 4).

In order to improve the systemic pharmacokinetics of a nucleic acid, there has been a method of changing the electron state of the functional group in the ionizable cationic lipid. NPL 5 compares the delivery efficiency of the structure in which the electron state in the vicinity of the ester bond moiety of the ionizable cationic lipid is changed by introducing an aromatic group to around the ester bond thereof. It is stated that as a result, an ionizable lipid containing an aromatic ester shows a higher nucleic acid delivery efficiency as compared to a normal alkyl ester. It can be understood that the introduction of the functional group is important in the in vivo clearance of the ionizable cationic lipid and the cellular uptake triggered by the self-destruction of lipid nanoparticles (LNP) having the ionizable cationic lipid incorporated therein.

The diseases targeted by nucleic acid therapeutics are diverse, and in order to establish the manifestation of therapeutic effects and the delivery of pharmaceuticals according to each disease, further improvement of the systemic pharmacokinetics and the delivery method directed to arbitrary organs have been investigated. While LNP are generally often used for the liver, the delivery thereof to other organs, such as the spleen and the kidney, is also required, and it is necessary therefor to increase the expression level in the target organ with respect to the expression level in the liver (see NPL 6).

As described above, in order to improve the nucleic acid delivery efficiency and to develop an ionizable lipid capable of being delivered to arbitrary organs, it is necessary to identify a tertiary amino group, a linker (i.e., a moiety that links the tertiary amino group and the lipid moiety), and the lipid moiety useful for these requirements, but such identification has not yet been achieved. Accordingly, the comprehensive and rapid screening of the aforementioned moieties is required, but the methods for synthesis thereof and for introducing the linker are still limited.

In view of the aforementioned problem, an object of the present invention is to provide an ionizable lipid that contains an ester moiety having high degradability and can be used as a nucleic acid delivery carrier having good intracellular pharmacokinetics. Another object thereof is to provide a lipid membrane structure, a nucleic acid transfer agent, and a pharmaceutical composition containing the ionizable lipid, a method for transferring a nucleic acid into cells or target cells, and a method for producing cellular medicine.

### Solution to Problem

As a result of earnest investigations by the present inventors, it has been found that the problem can be solved by an ionizable lipid used for delivering pharmaceuticals, containing at least one linear or cyclic tertiary amino group, at least one phenyl group having an ester bond at an o-position, and an aliphatic hydrocarbon group having 4 or more carbon atoms, and thus the present invention has been completed.

Specifically, the present invention provides the following items [1] to [14].

[1] An ionizable lipid used for delivering pharmaceuticals, containing at least one linear or cyclic tertiary amino group, at least one phenyl group having an ester bond at an o-position, and an aliphatic hydrocarbon group having 4 or more carbon atoms.

[2] The ionizable lipid according to the item [1], in which the ionizable lipid is selected from the group consisting of compounds represented by the following formulae (1) and (2):

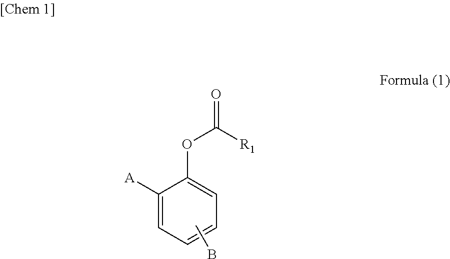

in which, in the formula (1),
A represents an aliphatic compound having 15 to 45 carbon atoms, having at least one linear or cyclic tertiary amino group, which may have at least any structure selected from the group consisting of a hydroxy group, an ether group, an ester group, a nitrile group, a amide group, a nitro group, a thioamide group, a sulfide group, a urea bond, a thioester bond, a thiocarbamate bond, a thiourea bond, a sulfonamide bond, a sulfonyl group, and a heterocyclic ring having at least any atom selected from nitrogen, oxygen, and sulfur, and may be bonded to an aromatic compound,
B represents (i) a hydrogen atom, (ii) an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur, or (iii) R₁′—CO—O—R₁₄—*, in which * represents a bonding position, R₁′ represents an aliphatic hydrocarbon group having 10 to 30 carbon atoms, and R₁₄ represents an aliphatic hydrocarbon group having 0 to 8 carbon atoms,
the group represented by B may be substituted at any one substitution position of four substitution positions of the aromatic ring shown in formula (1), or may be substituted at two or more substitution positions thereof, and in the case where the groups represented by B are substituted at two or more substitution positions, the multiple groups may be the same as or different from each other, and the groups at the adjacent two substitution positions may form a ring, and
R₁ represents an aliphatic hydrocarbon group having 10 to 30 carbon atoms,

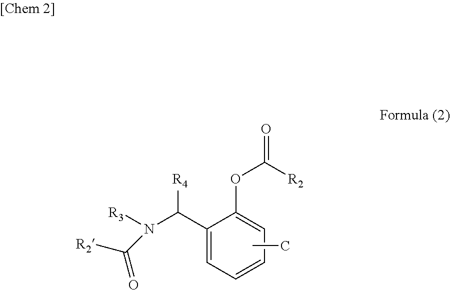

in which, in the formula (2),
R₂ and R₂′ each independently represent an aliphatic hydrocarbon group having 10 to 30 carbon atoms,
R₃ represents an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound,
R₄ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound, and
C represents R₁₂-R₁₃—*, in which * represents a bonding position, R₁₂ represents a tertiary amino group having 2 to 6 carbon atoms, and R₁₃ represents an aliphatic compound having 2 to 30 carbon atoms, which may have a hydroxy group, and may have an ester bond.

[3] The ionizable lipid according to the item [1] or [2], in which the ionizable lipid is selected from the group consisting of compounds represented by the following formulae (3) to (6):

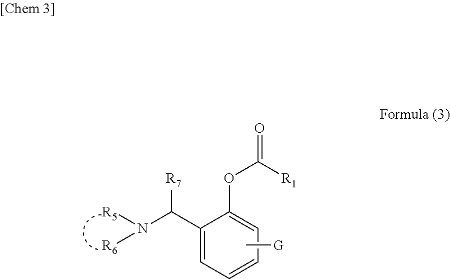

in which, in the formula (3),
R₁ has the same meaning as in the formula (1),
R₅ and R₆ each independently represent an aliphatic compound having 1 to 30 carbon atoms, 0 to 5 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, and a heterocyclic ring having at least any atom selected from nitrogen, oxygen, and sulfur, and R₅ and R₆ each may be independent from each other, or may be connected to each other to form a ring,
R₇ represents an aliphatic compound having 2 to 30 carbon atoms, 2 to 10 oxygen atoms, 0 to 5 nitrogen atoms, and 0 to 2 sulfur atoms, having an ester bond, which may have at least any structure selected from the group consisting of a hydroxy group, an ether group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound,
G represents a hydrogen atom or an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur, and
the group represented by G may be substituted at any one substitution position of four substitution positions of the aromatic ring shown in formula (3), or may be substituted at two or more substitution positions thereof, and in the case where the groups represented by G are substituted at two or more substitution positions, the multiple groups may be the same as or different from each other, and the groups at the adjacent two substitution positions may form a ring,

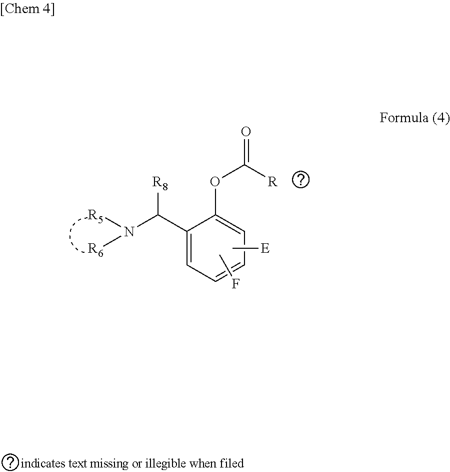

in which in the formula (4),
R₁ has the same meaning as in the formula (1),
R₅ and R₆ have the same meanings as in the formula (3),
R₈ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound,
E represents a hydrogen atom, a nitro group, a methoxy group, a nitrile group, a methyl group, a tert-butyl group, or a 1,1-dimethylpropyl group, and
F represents R₁′—CO—O—R₁₄—*, in which * represents a bonding position, R₁′ has the same meaning as in the formula (1), and R₁₄ represents an aliphatic hydrocarbon group having 0 to 8 carbon atoms,

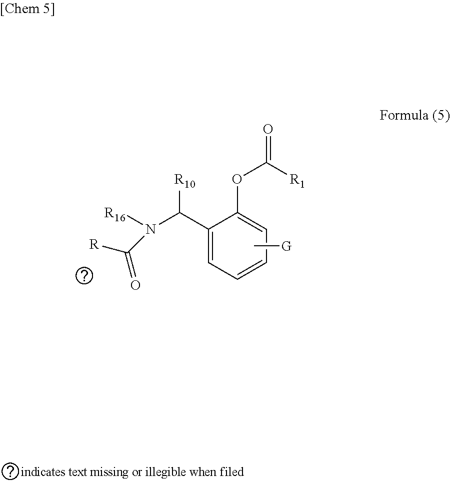

in which, in the formula (5),
R₁ has the same meaning as in the formula (1),
R₉ represents an aliphatic hydrocarbon group having 10 to 30 carbon atoms,
R₁₀ represents an aliphatic compound having at least one linear or cyclic tertiary amine structure, having 2 to 15 carbon atoms, 0 to 10 oxygen atoms, and 1 to 5 nitrogen atoms, which may have at least any functional group selected from the group consisting of an ester group, an amide group, a carbamate group, an ether group, a urea group, a thioamide group, a sulfide group, and a sulfonamide group, and may be bonded to an aromatic compound,
R₁₆ represents an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound, and
G has the same meaning as in the formula (3),

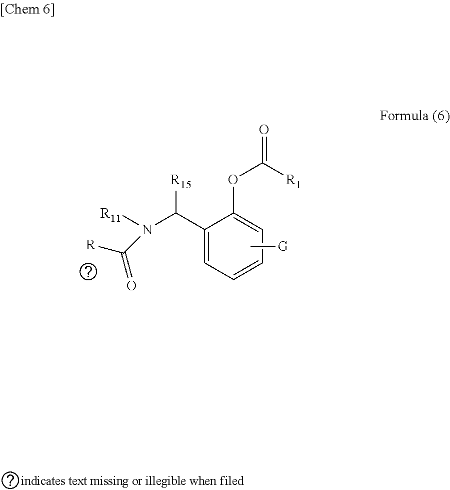

in which, in the formula (6),
R₁ has the same meaning as in the formula (1),
R₉ has the same meaning as in the formula (5),
R₁₁ represents an aliphatic compound having at least one linear or cyclic tertiary amine structure, having 1 to 15 carbon atoms, which may be bonded to an aromatic compound,
R₁₅ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound, and
G has the same meaning as in the formula (3).

[4] The ionizable lipid according to the item [2], in which the compound represented by the formula (2) satisfies the following requirements:
R₂ and R₂′ each independently represent an alkyl group having 10 to 30 carbon atoms, an alkenyl group having 10 to 30 carbon atoms, or an alkynyl group having 10 to 30 carbon atoms,
R₃ represents an aliphatic compound having 3 to 13 carbon atoms, 0 to 4 oxygen atoms, 0 to 1 nitrogen atom, and 0 to 1 sulfur atom, which may have at least any structure selected from the group consisting of an ether group, an ester group, a nitrile group, an amide group, a nitro group, a sulfide group, a sulfonamide group, a sulfonyl group, and a heterocyclic ring having at least any atom selected from nitrogen, oxygen, and sulfur, and may be bonded to an aromatic compound,
R₄ represents an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound, and
C represents any of structures represented by the following formulae (7) to (9):

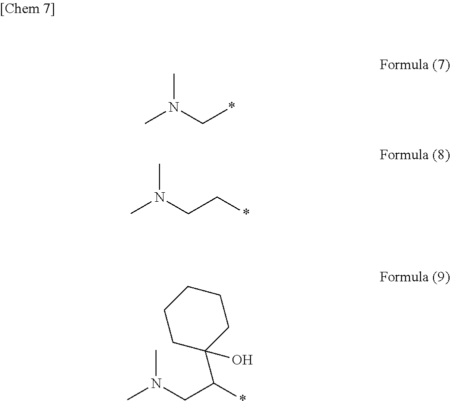

in which, in the formulae (7) to (9), * represents a bonding position.

[5] The ionizable lipid according to the item [3], in which the compounds represented by the formulae (3) to (6) satisfy the following requirements:
R₁ and R₁′ each independently represent an alkyl group having 10 to 30 carbon atoms, an alkenyl group having 10 to 30 carbon atoms, or an alkynyl group having 10 to 30 carbon atoms,
R₅ and R₆ each independently represent an aliphatic compound having 1 to 15 carbon atoms, 0 to 2 oxygen atoms, 0 to 1 nitrogen atom, and 0 to 1 sulfur atom, which may form a ring, may contain an unsaturated bond, may have at least any structure selected from the group consisting of an ether group, an ester group, a nitrile group, an amide group, a nitro group, a sulfide group, a sulfonamide group, and a heterocyclic ring having at least any atom selected from nitrogen, oxygen, and sulfur, and R₅ and R₆ each may be independent from each other, or may be connected to each other to form a ring,
R₇ represents an aliphatic compound having 5 to 30 carbon atoms, 2 to 4 oxygen atoms, 0 to 1 nitrogen atom, and 0 to 1 sulfur atom, having an ester bond, which may contain an unsaturated bond, may have a branch, may form a ring, and may have at least any structure selected from the group consisting of an aromatic ring, an ether group, and a heterocyclic ring having at least any atom selected from nitrogen, oxygen, and sulfur,
R₈ represents an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound,
R₁₀ represents any of structures represented by the following formulae (10) to (14):

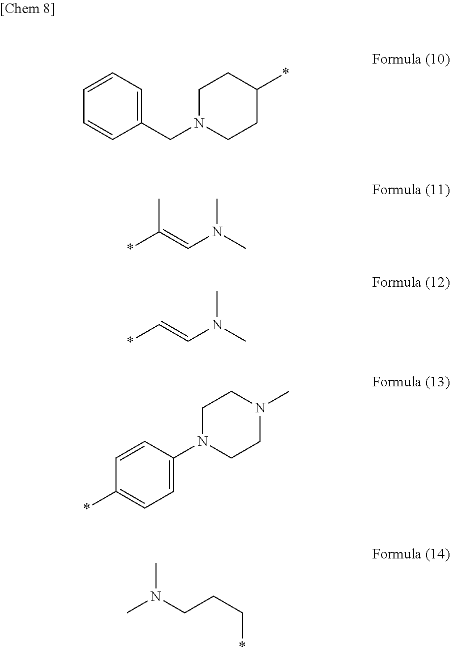

in which, in the formulae (10) to (14), * represents a bonding position,
R₁₄ represents an alkylene group having 0 to 8 carbon atoms, an alkenediyl group having 0 to 8 carbon atoms, or an alkynediyl group having 0 to 8 carbon atoms,
R₁₅ represents an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound, and
R₁₆ represents an aliphatic compound having 3 to 13 carbon atoms, 0 to 4 oxygen atoms, 0 to 1 nitrogen atom, and 0 to 1 sulfur atom, which may have at least any structure selected from the group consisting of an ether group, an ester group, a nitrile group, an amide group, a nitro group, a sulfide group, a sulfonamide group, a sulfonyl group, and a heterocyclic ring having at least any atom selected from nitrogen, oxygen, and sulfur, and may be bonded to an aromatic compound.

[6] A lipid membrane structure containing the ionizable lipid according to any one of the items [1] to [5] as a constitutional lipid of the membrane.

[7] The lipid membrane structure according to the item [6], in which the lipid membrane structure further contains a nucleic acid.

[8] A nucleic acid transfer agent containing the ionizable lipid according to any one of the items [1] to [5].

[9] The nucleic acid transfer agent according to the item [8], in which the nucleic acid transfer agent further contains a nucleic acid.

[10] A pharmaceutical composition containing the ionizable lipid according to any one of the items [1] to [5].

[11] The pharmaceutical composition according to the item [10], in which the pharmaceutical composition further contains a nucleic acid.

[12] A method for transferring a nucleic acid contained in a nucleic acid transfer agent into cells, including bringing the cells into contact with the nucleic acid transfer agent according to the item [8] or [9] in vitro.

[13] A method for transferring a nucleic acid contained in a nucleic acid transfer agent to target cells of a living body, including administrating the nucleic acid transfer agent according to the item [8] or [9] into the living body.

[14] A method for producing a cellular medicine containing cells that express a gene in a nucleic acid, including bringing cells into contact with the nucleic acid transfer agent according to the item [8] or [9] to transfer the nucleic acid contained in the nucleic acid transfer agent into the cells.

### Advantageous Effects of Invention

The present invention relates to an ionizable lipid having a tertiary amino group, an aromatic ester, and a lipid moiety, and a lipid membrane structure containing the same. The ionizable lipid of the present invention can form a lipid membrane structure, and can form a nucleic acid transfer agent containing the ionizable lipid. Furthermore, the ionizable lipid of the present invention contains an aromatic ester having high degradability, and thereby the particles can be efficiently destructed to achieve a high delivery efficiency. Moreover, development of the ionizable lipid that is useful for arbitrary organs can be provided by synthesizing an ionizable lipid capable of comprehensively changing the structure including the tertiary amino group, the linker, and the lipid moiety, and preparing and evaluating the LNP.

### DETAILED DESCRIPTION OF THE INVENTION

In the description herein and the scope of claim (which may be abbreviated as the “description herein”), the “ionizable lipid represented by the formula (1)” may be abbreviated as the ionizable lipid (1). The ionizable lipids and the compounds represented by the other formulae may also be abbreviated as similar to the “ionizable lipid represented by the formula (1)”.

In the description herein, “*” and the like represent a bonding position, but not a carbon atom as described above, and the structure including two or more “*” is in accordance with the description in the table.

The ionizable lipid of the present invention is an ionizable lipid used for delivering pharmaceuticals, containing at least one linear or cyclic tertiary amino group, at least one phenyl group having an ester bond at an o-position, and an aliphatic hydrocarbon group having 4 or more carbon atoms.

The expression “having an ester bond at an o-position” herein means that the ester bond exists at the o-position with respect to the other group bonded to the phenyl group than the group having the ester bond. The “other group than the group having the ester bond” may be the linear or cyclic tertiary amino group, or may be the aliphatic hydrocarbon group having 4 or more carbon atoms.

First, the linear or cyclic tertiary amino group is described.

In the description herein, the linear tertiary amino group means a tertiary amino group having linear chains that are independent from each other.

In the description herein, the cyclic tertiary amino group means a tertiary amino group containing an amine and forming one or more cyclic ring structure.

In the description herein, the aliphatic compound means an alkyl group, an alkenyl group, and an alkynyl group, which may have a hydrocarbon ring group, a hydroxy group, a carbonyl group, a carboxylic acid group, and a halogen atom.

In the description herein, the aliphatic hydrocarbon group means an alkyl group, an alkenyl group, an alkynyl group and an alkylene group that do not contain a hetero atom.

In the description herein, the alkyl group may be either linear or branched. Examples of the alkyl group include a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, a eicosyl group, a heneicosyl group, a docosyl group, a tricosyl group, a tetracosyl group, a pentacosyl group, a hexacosyl group, a heptacosyl group, an octacosyl group, a nonacosyl group, a triacontyl group, a hentriacontyl group, a dotriacontyl group, a tritriacontyl group, a tetratriacontyl group, a pentatriacontyl group, a hexatriacontyl group, a tetracontyl group, a hentetracontyl group, a dotetracontyl group, a tritetracontyl group, and a tetratetracontyl group.

In the description herein, the alkenyl group may be either linear or branched. In the description herein, the number of the olefinic carbon-carbon double bond in the alkenyl group may be only 1, or may be 2 or more. Examples of the alkenyl group include an ethenyl group, a propenyl group, a butenyl group, a pentenyl group, a hexenyl group, a heptenyl group, an octenyl group, a nonenyl group, a decenyl group, an undecenyl group, a dodecenyl group, a tridecenyl group, a tetradecenyl group, a pentadecenyl group, a hexadecenyl group, a heptadecenyl group, an octadecenyl group, a nonadecenyl group, an eicosenyl group, a heneicosenyl group, a docosenyl group, a tricosenyl group, a tetracosenyl group, a pentacosenyl group, a hexacosenyl group, a heptacosenyl group, an octacosenyl group, a nonacosenyl group, a triacontenyl group, a hentriacontenyl group, and a dotriacontenyl group.

In the description herein, the alkynyl group may be either linear or branched. In the description herein, the number of the carbon-carbon triple bond in the alkynyl group may be only 1, or may be 2 or more. Examples of the alkynyl group include an ethynyl group, a propynyl group, a butynyl group, a pentynyl group, a hexynyl group, a heptynyl group, an octynyl group, a nonynyl group, a decynyl group, an undecynyl group, a dodecynyl group, a tridecynyl group, a tetradecynyl group, a pentadecynyl group, a hexadecynyl group, a heptadecynyl group, an octadecynyl group, a nonadecynyl group, an eicosynyl group, a heneicosynyl group, a docosynyl group, a tricosynyl group, a tetracosynyl group, a pentacosynyl group, a hexacosynyl group, a heptacosynyl group, an octacosynyl group, a nonacosynyl group, a triacontynyl group, a hentriacontynyl group, and a dotriacontynyl group.

In the description herein, examples of the halogen atom include a fluorine atom, a chlorine atom, a bromine atom, and an iodine atom.

In the description herein, the alkylene group may be either linear or branched. Examples of the alkylene group include a methylene group, an ethylene group, a trimethylene group (—(CH₂)₃—), a propylene group (—CH(CH₃)CH₂—, —CH₂CH(CH₃)—), a tetramethylene group (—(CH₂)₄—), a butylene group (—CH(C₂H₅)CH₂—, —CH₂CH(C₂H₅)—), a pentamethylene group (—(CH₂)₅—), a hexamethylene group (—(CH₂)₆—), a heptamethylene group (—(CH₂)₇—), an octamethylene group (—(CH₂)₈—), a nonamethylene group (—(CH₂)₉—), and a decamethylene group (—(CH₂)₁₀—). In the formulae above, “—” represents a single bond.

In the description herein, the alkenediyl group means a divalent group having a structure obtained by removing two hydrogen atoms from an alkene. In the description herein, the alkenediyl group may be either linear or branched. In the description herein, the number of the olefinic carbon-carbon double bond in the alkene or the alkenediyl group may be only 1, or may be 2 or more. Examples of the alkenediyl group include an ethenediyl group, a propenediyl group, a butenediyl group, a pentenediyl group, a hexenediyl group, a heptenediyl group, an octenediyl group, a nonenediyl group, and a decenediyl group. In the description herein, the “(compound)+diyl group” (e.g., ethendiyl group) means a divalent group having a structure obtained by removing two hydrogen atoms from the compound.

In the description herein, the alkynediyl group means a divalent group having a structure obtained by removing two hydrogen atoms from an alkyne. In the description herein, the alkynediyl group may be either linear or branched. In the description herein, the number of the carbon-carbon triple bond in the alkyne or the alkynediyl group may be only 1, or may be 2 or more. Examples of the alkynediyl group include an ethynediyl group, a propynediyl group, a butynediyl group, a pentynediyl group, a hexynediyl group, a heptynediyl group, an octynediyl group, a nonynediyl group, and a decynediyl group.

In the description herein, the hydrocarbon ring group means a cyclic group. Examples of the hydrocarbon ring group having 3 to 12 carbon atoms include a cycloalkyl group and an adamantyl group. Examples of the cycloalkyl group include a cyclopropyl group, a cyclobutyl group, a cyclopentyl group, a cyclohexyl group, a cycloheptyl group, and a cyclooctyl group.

In the description herein, the aromatic compound means an aromatic hydrocarbon ring or an aromatic heterocyclic ring. Examples of the aromatic hydrocarbon ring include a benzene ring, a naphthalene ring, and an anthracene ring. Examples of the aromatic heterocyclic ring include an imidazole ring, a pyrazole ring, an oxazole ring, an isoxazole ring, a thiazole ring, an isothiazole ring, a triazine ring, a pyrrole ring, a furan-thiophene ring, a pyrimidine ring, a pyridazine ring, a pyrazine ring, a pyridine ring, a purine ring, a pteridine ring, a benzimidazole ring, an indole ring, a benzofuran ring, a quinazoline ring, a phthalazine ring, a quinoline ring, an isoquinoline ring, a coumarin ring, a chromone ring, a benzodiazepine ring, a phenoxazine ring, a phenothiazine ring, and an acridine ring. These aromatic rings each may be substituted by a substituent, and examples of the substituent include an acyl group having 2 to 4 carbon atoms, an alkoxycarbonyl group having 2 to 4 carbon atoms, a carbamoyl group having 2 to 4 carbon atoms, an acyloxy group having 2 to 4 carbon atoms, an acylamino group having 2 to 4 carbon atoms, an alkoxycarbonylamino group having 2 to 4 carbon atoms, a fluorine atom, a chlorine atom, a bromine atom, an iodine atom, an alkylsulfanyl group having 1 to 4 carbon atoms, an alkylsulfonyl group having 1 to 4 carbon atoms, an arylsulfonyl group having 6 to 10 carbon atoms, a nitro group, a trifluoromethyl group, a cyano group, an alkyl group having 1 to 4 carbon atoms, a ureido group having 1 to 4 carbon atoms, an alkoxy group having 1 to 4 carbon atoms, an aryl group having 6 to 10 carbon atoms, and an aryloxy group having 6 to 10 carbon atoms.

In the description herein, the ester bond means —CO—O— or —O—CO— (in which “—” represents a single bond). The ester bond and the ester group have the same meaning.

In the description herein the amide bond means —CO—NH— or —NH—CO— (in which “—” represents a single bond). The amide bond and the amide group have the same meaning.

In the description herein, the carbamate bond means —O—CO—NH— or —NH—CO—O— (in which “—” represents a single bond). The carbamate bond and the carbamate group have the same meaning.

In the description herein, the ether bond means —O— (in which “—” represents a single bond). The ether bond and the ether group have the same meaning.

In the description herein, the urea bond means —NH—CO—NH— (in which “—” represents a single bond). The urea bond and the urea group have the same meaning.

In the description herein, the carbonate bond means —O—CO—O— (in which “—” represents a single bond). The carbonate bond and the carbonate group have the same meaning.

The ionizable lipid of the present invention is preferably a compound represented by the following formula (1) or (2).

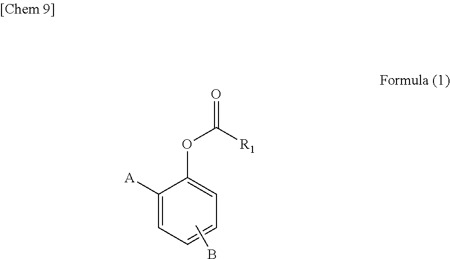

In the formula (1),
A represents an aliphatic compound having 15 to 45 carbon atoms, having at least one linear or cyclic tertiary amino group, which may have at least any structure selected from the group consisting of a hydroxy group, an ether group, an ester group, a nitrile group, a amide group, a nitro group, a thioamide group, a sulfide group, a urea bond, a thioester bond, a thiocarbamate bond, a thiourea bond, a sulfonamide bond, a sulfonyl group, and a heterocyclic ring having at least any atom selected from nitrogen, oxygen, and sulfur, and may be bonded to an aromatic compound,
B represents (i) a hydrogen atom, (ii) an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur, or (iii) R₁′—CO—O—R₁₄—*, in which * represents a bonding position, R₁′ represents an aliphatic hydrocarbon group having 10 to 30 carbon atoms, and R₁₄ represents an aliphatic hydrocarbon group having 0 to 8 carbon atoms,
the group represented by B may be substituted at any one substitution position of four substitution positions of the aromatic ring shown in formula (1), or may be substituted at two or more substitution positions thereof, and in the case where the groups represented by B are substituted at two or more substitution positions, the multiple groups may be the same as or different from each other, and the groups at the adjacent two substitution positions may form a ring, and
R₁ represents an aliphatic hydrocarbon group having 10 to 30 carbon atoms.

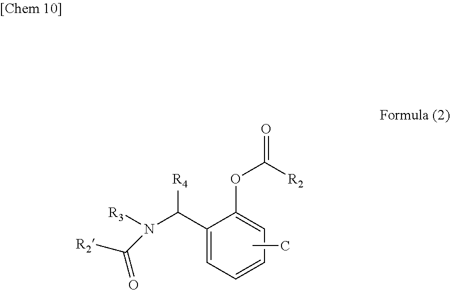

In the formula (2),
R₂ and R₂′ each independently represent an aliphatic hydrocarbon group having 10 to 30 carbon atoms,
R₃ represents an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound,
R₄ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound, and
C represents R₁₂—R₁₃—*, in which * represents a bonding position, R₁₂ represents a tertiary amino group having 2 to 6 carbon atoms, and R₁₃ represents an aliphatic compound having 2 to 30 carbon atoms, which may have a hydroxy group, and may have an ester bond.

The ionizable lipid of the present invention is more preferably a compound represented by any of the following formulae (3) to (6).

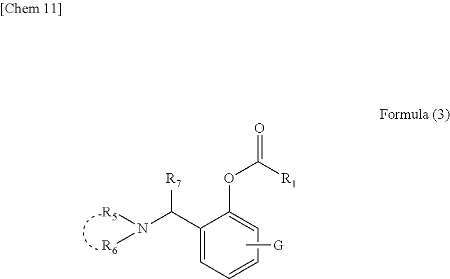

In the formula (3),
R₁ has the same meaning as in the formula (1),
R₅ and R₆ each independently represent an aliphatic compound having 1 to 30 carbon atoms, 0 to 5 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, and a heterocyclic ring having at least any atom selected from nitrogen, oxygen, and sulfur, and R₅ and R₆ each may be independent from each other, or may be connected to each other to form a ring,
R₇ represents an aliphatic compound having 2 to 30 carbon atoms, 2 to 10 oxygen atoms, 0 to 5 nitrogen atoms, and 0 to 2 sulfur atoms, having an ester bond, which may have at least any structure selected from the group consisting of a hydroxy group, an ether group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound,
G represents a hydrogen atom or an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur, and
the group represented by G may be substituted at any one substitution position of four substitution positions of the aromatic ring shown in formula (3), or may be substituted at two or more substitution positions thereof, and in the case where the groups represented by G are substituted at two or more substitution positions, the multiple groups may be the same as or different from each other, and the groups at the adjacent two substitution positions may form a ring.

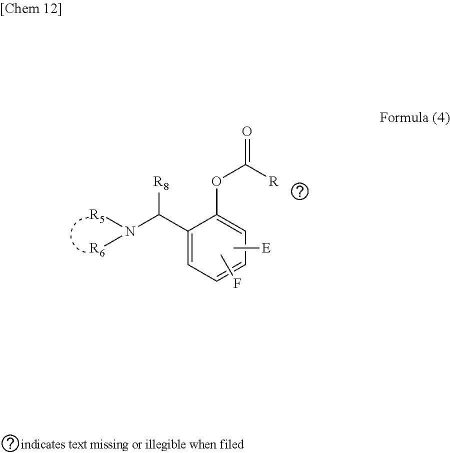

In the formula (4),
R₁ has the same meaning as in the formula (1),
R₅ and R₆ have the same meanings as in the formula (3),
R₈ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound,
E represents a hydrogen atom, a nitro group, a methoxy group, a nitrile group, a methyl group, a tert-butyl group, or a 1,1-dimethylpropyl group, and
F represents R₁′—CO—O—R₁₄—*, in which * represents a bonding position, R₁′ has the same meaning as in the formula (1), and R₁₄ represents an aliphatic hydrocarbon group having 0 to 8 carbon atoms.

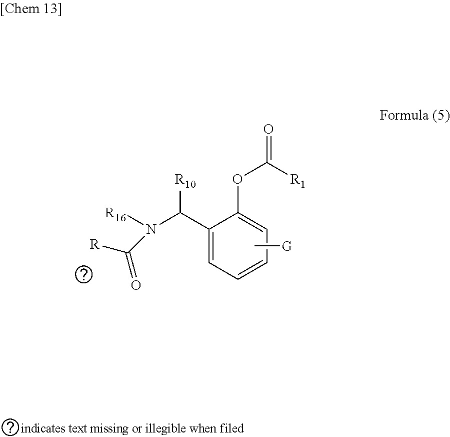

In the formula (5),
R₁ has the same meaning as in the formula (1),
R₉ represents an aliphatic hydrocarbon group having 10 to 30 carbon atoms,
R₁₀ represents an aliphatic compound having at least one linear or cyclic tertiary amine structure, having 2 to 15 carbon atoms, 0 to 10 oxygen atoms, and 1 to 5 nitrogen atoms, which may have at least any functional group selected from the group consisting of an ester group, an amide group, a carbamate group, an ether group, a urea group, a thioamide group, a sulfide group, and a sulfonamide group, and may be bonded to an aromatic compound,
R₁₆ represents an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound, and
G has the same meaning as in the formula (3).

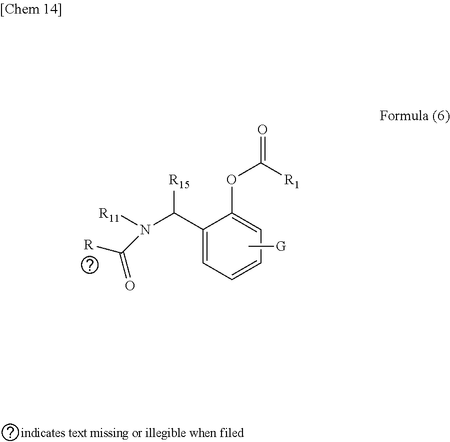

In the formula (6),
R₁ has the same meaning as in the formula (1),
R₉ has the same meaning as in the formula (5),
R₁₁ represents an aliphatic compound having at least one linear or cyclic tertiary amine structure, having 1 to 15 carbon atoms, which may be bonded to an aromatic compound,
R₁₅ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound, and
G has the same meaning as in the formula (3).

The formula (1) and the following formulae are described in order below.

The compound represented by the formula (1) is preferably a compound represented by any of the formulae (3), (4), (5), and (6).

R₂ and R₂′ in the formula (2) each independently represent an aliphatic hydrocarbon group having 10 to 30 carbon atoms, as described above.

R₂ and R₂′ in the formula (2) each preferably independently represent an alkyl group having 10 to 30 carbon atoms, an alkenyl group having 10 to 30 carbon atoms, or an alkynyl group having 10 to 30 carbon atoms, each more preferably independently represent an alkyl group having 10 to 30 carbon atoms or an alkenyl group having 10 to 30 carbon atoms, and each further preferably independently represent an alkyl group having 10 to 20 carbon atoms or an alkenyl group having 10 to 20 carbon atoms.

R₃ in the formula (2) and R₁₆ in the formula (5) each represent an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound.

R₃ in the formula (2) and R₁₆ in the formula (5) each preferably represent an aliphatic compound having 3 to 13 carbon atoms, 0 to 4 oxygen atoms, 0 to 1 nitrogen atom, and 0 to 1 sulfur atom, which may have at least any structure selected from the group consisting of an ether group, an ester group, a nitrile group, an amide group, a nitro group, a sulfide group, a sulfonamide group, a sulfonyl group, and a heterocyclic ring having at least any atom selected from nitrogen, oxygen, and sulfur, and may be bonded to an aromatic compound.

R₃ in the formula (2) and R₁₆ in the formula (5) each are more preferably selected from the structures shown in Tables 1-1 to 1-3 below. In Tables 1-1 to 1-3, * represents a bonding point.

TABLE 1-1

| | |
|---|---|
| 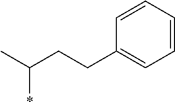 [SMILES: *C(C)CCc1ccccc1] | structure 1 |
| 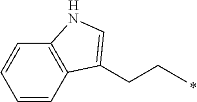 | structure 2 |
| 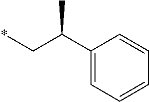 [SMILES: *C[C@@H](C)c1ccccc1] | structure 3 |
| 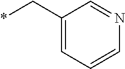 [SMILES: *Cc1cccnc1] | structure 4 |
| 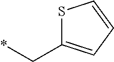 [SMILES: *Cc1cccs1] | structure 5 |
| 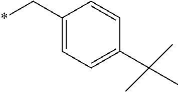 [SMILES: *Cc1ccc(C(C)(C)C)cc1] | structure 6 |
| 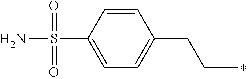 | structure 7 |
| 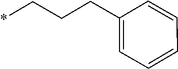 [SMILES: *CCCc1ccccc1] | structure 8 |
| 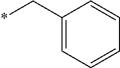 [SMILES: *Cc1ccccc1] | structure 9 |
| 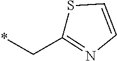 [SMILES: *Cc1nccs1] | structure 10 |
| 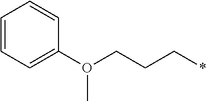 | structure 11 |
| 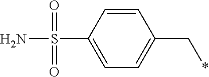 [SMILES: *Cc1ccc(S(N)(=O)=O)cc1] | structure 12 |
| 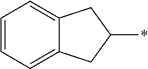 [SMILES: *C1Cc2ccccc2C1] | structure 13 |
| 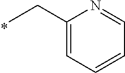 | structure 14 |
| 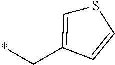 [SMILES: *Cc1ccsc1] | structure 15 |
| 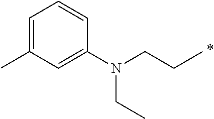 | structure 16 |
| 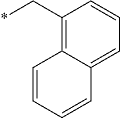 [SMILES: *Cc1cccc2ccccc12] | structure 17 |
| 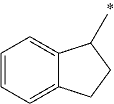 | structure 18 |
| 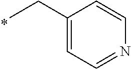 | structure 19 |
| 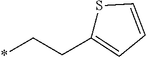 [SMILES: *CCc1cccs1] | structure 20 |
| 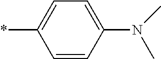 [SMILES: *c1ccc(N(C)C)cc1] | structure 21 |
| 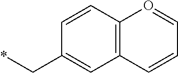 | structure 22 |
| 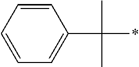 [SMILES: *C(C)(C)c1ccccc1] | structure 23 |
| 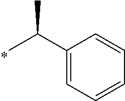 | structure 24 |
| 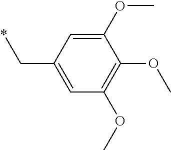 [SMILES: *Cc1cc(OC)c(OC)c(OC)c1] | structure 25 |
| 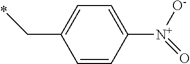 [SMILES: *Cc1ccc([N+](=O)[O-])cc1] | structure 26 |
| 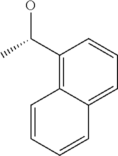 | structure 27 |
| 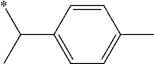 [SMILES: *C(C)c1ccc(C)cc1] | structure 28 |
| 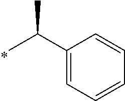 | structure 29 |
| 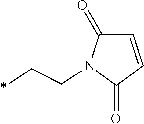 [SMILES: *CCN1C(=O)C=CC1=O] | structure 30 |
| 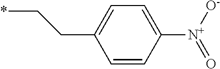 [SMILES: *CCc1ccc([N+](=O)[O-])cc1] | structure 31 |
| 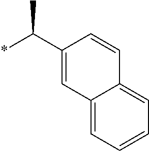 [SMILES: *[C@@H](C)c1ccc2ccccc2c1] | structure 32 |
| 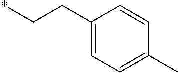 [SMILES: *CCc1ccc(C)cc1] | structure 33 |
| 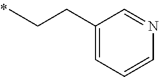 | structure 34 |
| 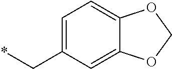 [SMILES: *Cc1ccc2c(c1)OCO2] | structure 35 |
| 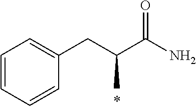 | structure 36 |
| 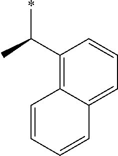 [SMILES: *[C@H](C)c1cccc2ccccc12] | structure 37 |
| 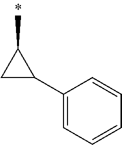 | structure 38 |
| 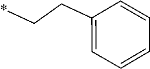 [SMILES: *CCc1ccccc1] | structure 39 |
| 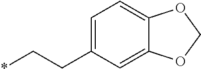 [SMILES: *CCc1ccc2c(c1)OCO2] | structure 40 |
| 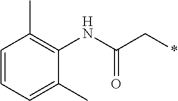 | structure 41 |
| 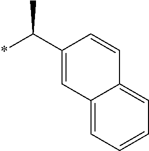 [SMILES: *[C@@H](C)c1ccc2ccccc2c1] | structure 42 |
| 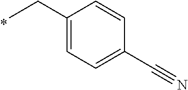 | structure 43 |
| 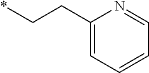 [SMILES: *CCc1ccccn1] | structure 44 |
| 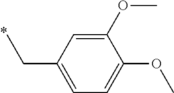 | structure 45 |
| 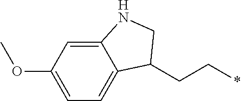 | structure 46 |
| 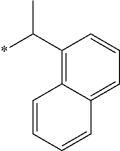 [SMILES: *C(C)c1cccc2ccccc12] | structure 47 |
| 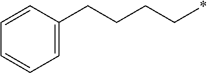 [SMILES: *CCCCc1ccccc1] | structure 48 |
| 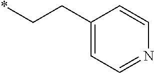 [SMILES: *CCc1ccncc1] | structure 49 |
| 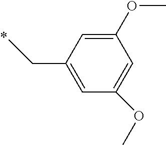 | structure 50 |
| 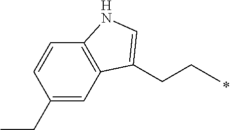 | structure 51 |
| 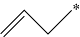 | structure 52 |
| 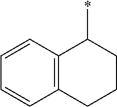 [SMILES: *C1CCCc2ccccc21] | structure 53 |
| 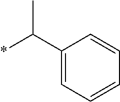 [SMILES: *C(C)c1ccccc1] | structure 54 |
| 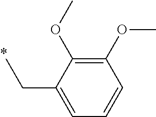 | structure 55 |
| 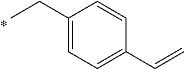 | structure 56 |
| 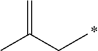 | structure 57 |
| 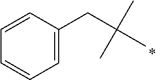 | structure 58 |
| 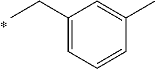 | structure 59 |
| 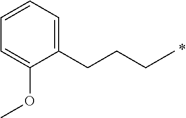 | structure 60 |
| 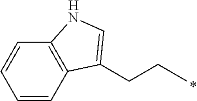 | structure 61 |
| 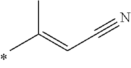 | structure 62 |
| 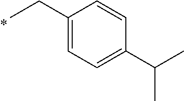 | structure 63 |
| 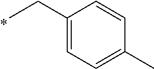 | structure 64 |
| 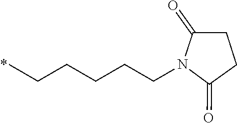 | structure 65 |
| 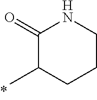 | structure 66 |
| 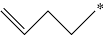 | structure 67 |
| 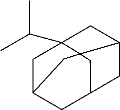 | structure 68 |
| 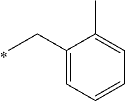 | structure 69 |
| 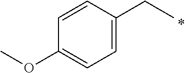 | structure 70 |

TABLE 1-2

| | |
|---|---|
| 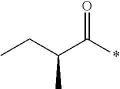 [SMILES: *C(=O)[C@@H](C)CC] | structure 71 |
| 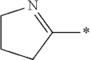 [SMILES: *C1=NCCC1] | structure 72 |
| 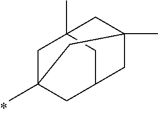 | structure 73 |
| 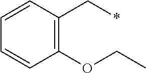 [SMILES: *Cc1ccccc1OCC] | structure 74 |
| 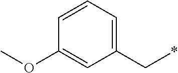 [SMILES: *Cc1cccc(OC)c1] | structure 75 |
| 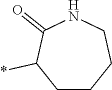 [SMILES: *C1CCCCNC1=O] | structure 76 |
| 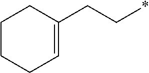 [SMILES: *CCC1=CCCCC1] | structure 77 |
| 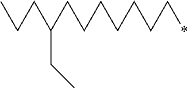 [SMILES: *CCCCCCCC(CC)CCC] | structure 78 |
| 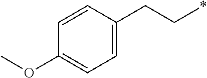 [SMILES: *CCc1ccc(OC)cc1] | structure 79 |
| 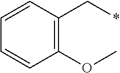 [SMILES: *Cc1ccccc1OC] | structure 80 |
| 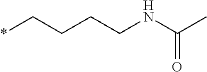 [SMILES: *CCCCNC(C)=O] | structure 81 |
| 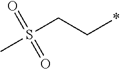 [SMILES: *CCS(C)(=O)=O] | structure 82 |
| 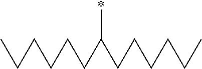 [SMILES: *C(CCCCCC)CCCCCC] | structure 83 |
| 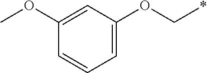 [SMILES: *COc1cccc(OC)c1] | structure 84 |
| 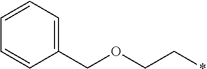 [SMILES: *CCOCc1ccccc1] | structure 85 |
| 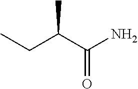 | structure 86 |
| 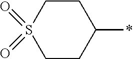 [SMILES: *C1CCS(=O)(=O)CC1] | structure 87 |
| 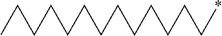 [SMILES: *CCCCCCCCCCCCC] | structure 88 |
| 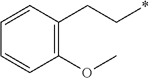 [SMILES: *CCc1ccccc1OC] | structure 89 |
| 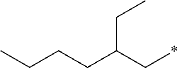 [SMILES: *CC(CC)CCCC] | structure 90 |
| 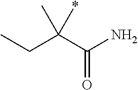 [SMILES: *C(C)(CC)C(N)=O] | structure 91 |
| 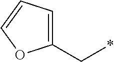 [SMILES: *Cc1ccco1] | structure 92 |
| 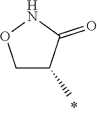 | structure 93 |
| 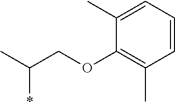 [SMILES: *C(C)COc1c(C)cccc1C] | structure 94 |
| 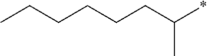 [SMILES: *C(C)CCCCCC] | structure 95 |
| 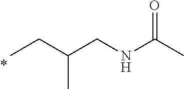 | structure 96 |
| 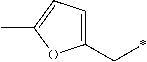 [SMILES: *Cc1ccc(C)o1] | structure 97 |
| 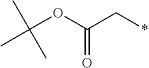 [SMILES: *CC(=O)OC(C)(C)C] | structure 98 |
| 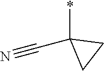 [SMILES: *C1(C#N)CC1] | structure 99 |
| 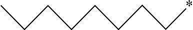 [SMILES: *CCCCCCCC] | structure 100 |
| 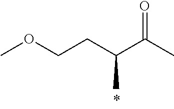 | structure 101 |
| 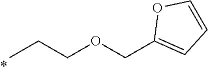 | structure 102 |
| 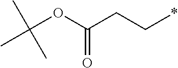 [SMILES: *CCC(=O)OC(C)(C)C] | structure 103 |
| 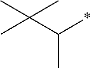 [SMILES: *C(C)C(C)(C)C] | structure 104 |
| 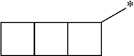 [SMILES: *C1CC2C3CCC3C12] | structure 105 |
| 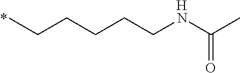 | structure 106 |
| 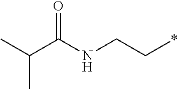 [SMILES: *CCNC(=O)C(C)C] | structure 107 |
| 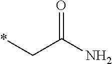 [SMILES: *CC(N)=O] | structure 108 |
| 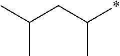 [SMILES: *C(C)CC(C)C] | structure 109 |
| 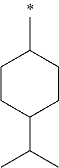 | structure 110 |
| 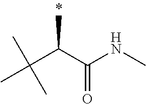 | structure 111 |
| 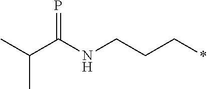 | structure 112 |
| 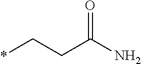 [SMILES: *CCC(N)=O] | structure 113 |
| 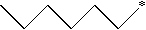 [SMILES: *CCCCCC] | structure 114 |
| 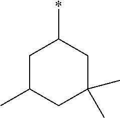 | structure 115 |
| 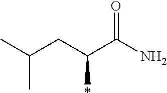 [SMILES: *[C@@H](CC(C)C)C(N)=O] | structure 116 |
| 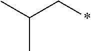 [SMILES: *CC(C)C] | structure 117 |
| 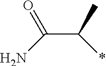 [SMILES: *[C@H](C)C(N)=O] | structure 118 |
| 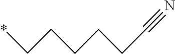 [SMILES: *CCCCCC#N] | structure 119 |
| 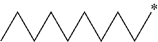 | structure 120 |
| 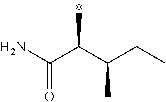 | structure 121 |
| 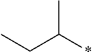 [SMILES: *C(C)CC] | structure 122 |
| 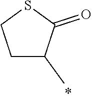 | structure 123 |
| 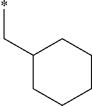 [SMILES: *CC1CCCCC1] | structure 124 |
| 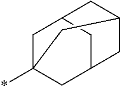 [SMILES: *C12CC3CC(CC(C3)C1)C2] | structure 125 |
| 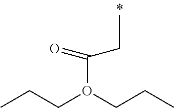 | structure 126 |
| 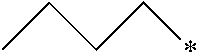 [SMILES: *CCCC] | structure 127 |
| 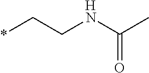 | structure 128 |
| 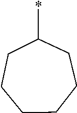 [SMILES: *C1CCCCCC1] | structure 129 |
| 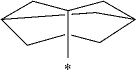 | structure 130 |
| 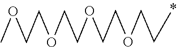 | structure 131 |
| 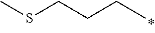 | structure 132 |
| 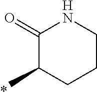 | structure 133 |
| 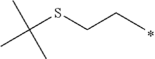 | structure 134 |
| 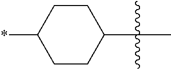 [SMILES: *C1CCC(*)CC1] | structure 135 |
| 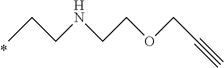 | structure 136 |
| 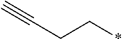 [SMILES: *CCC#C] | structure 137 |
| 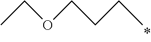 | structure 138 |
| 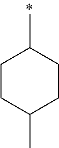 | structure 139 |
| 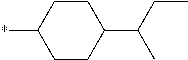 [SMILES: *C1CCC(C(C)CC)CC1] | structure 140 |

TABLE 1-3

| | |
|---|---|
| 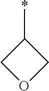 [SMILES: *C1COC1] | structure 141 |
| 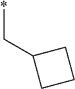 [SMILES: *CC1CCC1] | structure 142 |
| 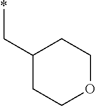 [SMILES: *CC1CCOCC1] | structure 143 |
| 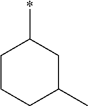 [SMILES: *C1CCCC(C)C1] | structure 144 |
| 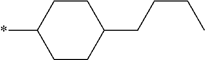 [SMILES: *C1CCC(CCCC)CC1] | structure 145 |
| 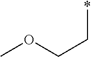 [SMILES: *CCOC] | structure 146 |
| 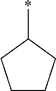 [SMILES: *C1CCCC1] | structure 147 |
| 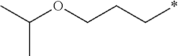 [SMILES: *CCCOC(C)C] | structure 148 |
| 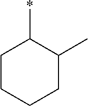 [SMILES: *C1CCCCC1C] | structure 149 |
| 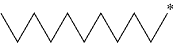 | structure 150 |
| 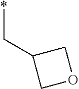 [SMILES: *CC1COC1] | structure 151 |
| 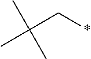 [SMILES: *CC(C)(C)C] | structure 152 |
| 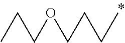 | structure 153 |
| 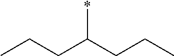 [SMILES: *C(CCC)CCC] | structure 154 |
| 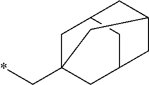 [SMILES: *CC12CC3CC(CC(C3)C1)C2] | structure 155 |
| 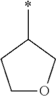 [SMILES: *C1CCOC1] | structure 156 |
| 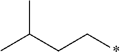 [SMILES: *CCC(C)C] | structure 157 |
| 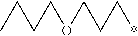 [SMILES: *CCCOCCCC] | structure 158 |
| 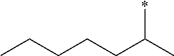 [SMILES: *C(C)CCCCC] | structure 159 |
| 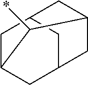 [SMILES: *C1C2CC3CC(C2)CC1C3] | structure 160 |
| 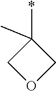 [SMILES: *C1(C)COC1] | structure 161 |
| 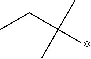 [SMILES: *C(C)(C)CC] | structure 162 |
| 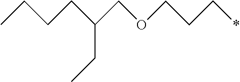 [SMILES: *CCCOCC(CC)CCCC] | structure 163 |
| 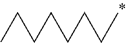 | structure 164 |
| 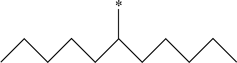 [SMILES: *C(CCCCC)CCCCC] | structure 165 |
| 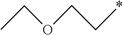 [SMILES: *CCOCC] | structure 166 |
| 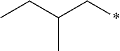 [SMILES: *CC(C)CC] | structure 167 |
| 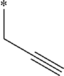 [SMILES: *CC#C] | structure 168 |
| 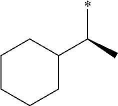 [SMILES: *[C@@H](C)C1CCCCC1] | structure 169 |
| 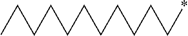 [SMILES: *CCCCCCCCCCC] | structure 170 |
| 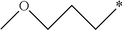 [SMILES: *CCCOC] | structure 171 |
| 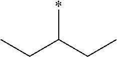 [SMILES: *C(CC)CC] | structure 172 |
| 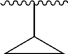 [SMILES: *C1CC1] | structure 173 |
| 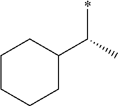 [SMILES: *[C@H](C)C1CCCCC1] | structure 174 |
| 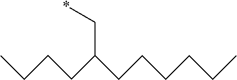 [SMILES: *CC(CCCC)CCCCCC] | structure 175 |
| 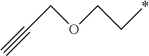 [SMILES: *CCOCC#C] | structure 176 |
| 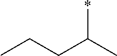 [SMILES: *C(C)CCC] | structure 177 |
| 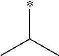 [SMILES: *C(C)C] | structure 178 |
| 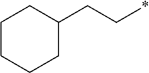 [SMILES: *CCC1CCCCC1] | structure 179 |
| 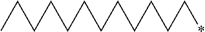 [SMILES: *CCCCCCCCCCCC] | structure 180 |
| 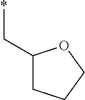 [SMILES: *CC1CCCO1] | structure 181 |
| 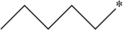 [SMILES: *CCCCC] | structure 182 |
| 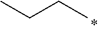 | structure 183 |
| 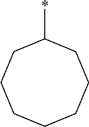 [SMILES: *C1CCCCCCC1] | structure 184 |
| 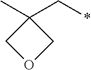 [SMILES: *CC1(C)COC1] | structure 185 |
| 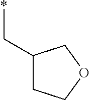 [SMILES: *CC1CCOC1] | structure 186 |
| 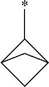 [SMILES: *C1C2CC1C2] | structure 187 |
| 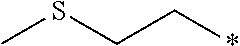 [SMILES: *CCSC] | structure 188 |
| 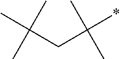 [SMILES: *C(C)(C)CC(C)(C)C] | structure 189 |
| 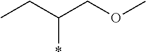 [SMILES: *C(CC)COC] | structure 190 |
| 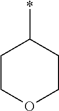 [SMILES: *C1CCOCC1] | structure 191 |
| 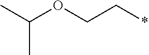 [SMILES: *CCOC(C)C] | structure 192 |
| 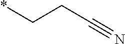 [SMILES: *CCC#N] | structure 193 |
| 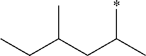 [SMILES: *C(C)CC(C)CC] | structure 194 |
| 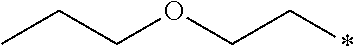 [SMILES: *CCOCCC] | structure 195 |
| 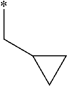 [SMILES: *CC1CC1] | structure 196 |
| 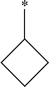 [SMILES: *C1CCC1] | structure 197 |
| 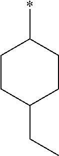 [SMILES: *C1CCC(CC)CC1] | structure 198 |
| 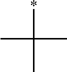 [SMILES: *C(C)(C)C] | structure 199 |
| 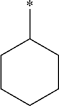 [SMILES: *C1CCCCC1] | structure 200 |

R₃ in the formula (2) and R₁₆ in the formula (5) each are most preferably selected from the structures shown in the Examples described hereinafter.

R₄ in the formula (2), R₈ in the formula (4), and R₁₅ in the formula (6) each represent an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound.

R₄ in the formula (2), R₈ in the formula (4), and R₁₅ in the formula (6) each preferably represent an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound.

R₄ in the formula (2), R₈ in the formula (4), and R₁₅ in the formula (6) each more preferably selected from the structures shown in Tables 2-1 to 2-4 below. In Tables 2-1 to 2-4, * represents a bonding point, and in the case where two bonding points exist, a condensed ring structure is formed at the adjacent two substitution positions.

TABLE 2-1

| | |
|---|---|
| 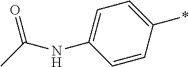 [SMILES: *c1ccc(NC(C)=O)cc1] | structure 1 |
| 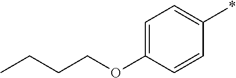 [SMILES: *c1ccc(OCCCC)cc1] | structure 2 |
| 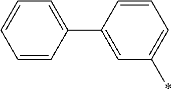 [SMILES: *c1cccc(-c2ccccc2)c1] | structure 3 |
| 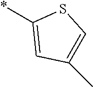 [SMILES: *c1cc(C)cs1] | structure 4 |
| 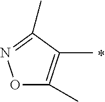 | structure 5 |
| 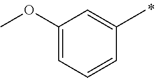 | structure 6 |
| 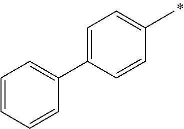 | structure 7 |
| 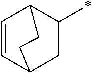 [SMILES: *C1CC2C=CC1CC2] | structure 8 |
| 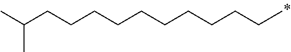 | structure 9 |
| 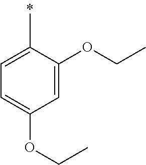 | structure 10 |
| 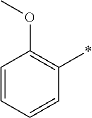 [SMILES: *c1ccccc1OC] | structure 11 |
| 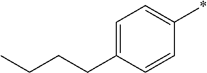 [SMILES: *c1ccc(CCCC)cc1] | structure 12 |
| 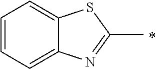 | structure 13 |
| 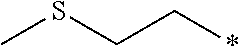 [SMILES: *CCSC] | structure 14 |
| 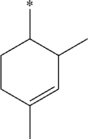 [SMILES: *C1CCC(C)=CC1C] | structure 15 |
| 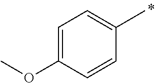 | structure 16 |
| 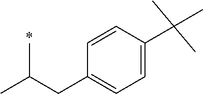 | structure 17 |
| 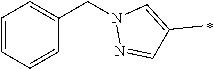 | structure 18 |
| 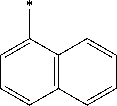 [SMILES: *c1cccc2ccccc12] | structure 19 |
| 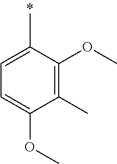 | structure 20 |
| 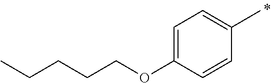 | structure 21 |
| 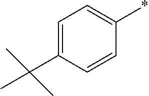 [SMILES: *c1ccc(C(C)(C)C)cc1] | structure 22 |
| 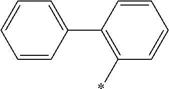 [SMILES: *c1ccccc1-c1ccccc1] | structure 23 |
| 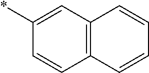 [SMILES: *c1ccc2ccccc2c1] | structure 24 |
| 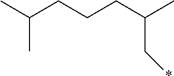 [SMILES: *CC(C)CCCC(C)C] | structure 25 |
| 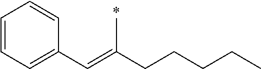 | structure 26 |
| 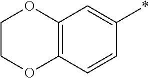 [SMILES: *c1ccc2c(c1)OCCO2] | structure 27 |
| 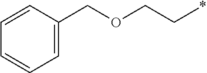 [SMILES: *CCOCc1ccccc1] | structure 28 |
| 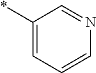 [SMILES: *c1cccnc1] | structure 29 |
| 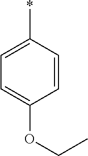 [SMILES: *c1ccc(OCC)cc1] | structure 30 |
| 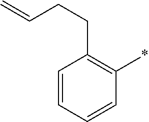 [SMILES: *c1ccccc1CCC=C] | structure 31 |
| 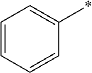 [SMILES: *c1ccccc1] | structure 32 |
| 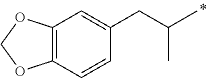 | structure 33 |
| 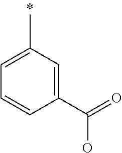 | structure 34 |
| 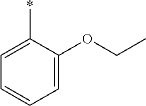 [SMILES: *c1ccccc1OCC] | structure 35 |
| 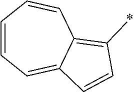 [SMILES: *c1ccc2cccccc1-2] | structure 36 |
| 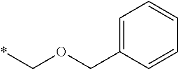 [SMILES: *COCc1ccccc1] | structure 37 |
| 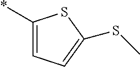 [SMILES: *c1ccc(SC)s1] | structure 38 |
| 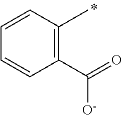 | structure 39 |
| 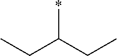 [SMILES: *C(CC)CC] | structure 40 |
| 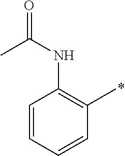 | structure 41 |
| 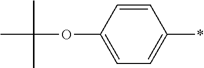 [SMILES: *c1ccc(OC(C)(C)C)cc1] | structure 42 |
| 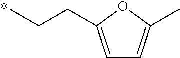 | structure 43 |
| 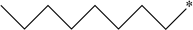 [SMILES: *CCCCCCCC] | structure 44 |
| 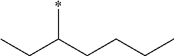 [SMILES: *C(CC)CCCC] | structure 45 |
| 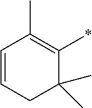 [SMILES: *C1=C(C)C=CCC1(C)C] | structure 46 |
| 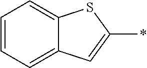 [SMILES: *c1cc2ccccc2s1] | structure 47 |
| 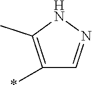 [SMILES: *c1cn[nH]c1C] | structure 48 |
| 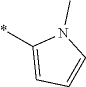 [SMILES: *c1cccn1C] | structure 49 |
| 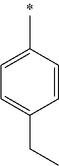 | structure 50 |
| 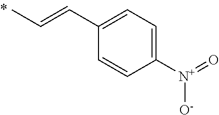 | structure 51 |
| 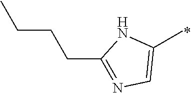 | structure 52 |
| 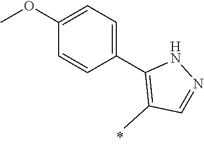 | structure 53 |
| 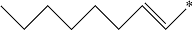 [SMILES: */C=C/CCCCCC] | structure 54 |
| 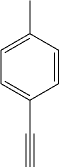 | structure 55 |
| 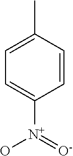 | structure 56 |
| 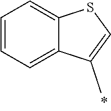 | structure 57 |
| 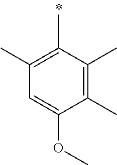 | structure 58 |
| 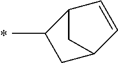 | structure 59 |
| 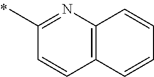 | structure 60 |
| 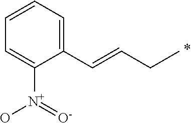 | structure 61 |
| 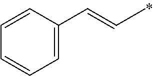 | structure 62 |
| 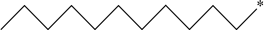 [SMILES: *CCCCCCCCCCC] | structure 63 |
| 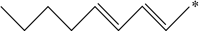 | structure 64 |
| 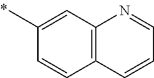 | structure 65 |
| 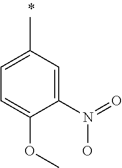 | structure 66 |
| 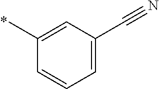 | structure 67 |
| 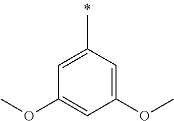 | structure 68 |
| 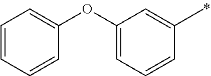 | structure 69 |
| 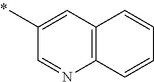 | structure 70 |

TABLE 2-2

| | |
|---|---|
| 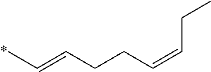 | structure 71 |
| 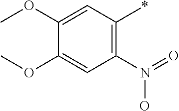 | structure 72 |
| 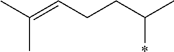 [SMILES: *C(C)CCC=C(C)C] | structure 73 |
| 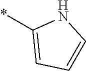 [SMILES: *c1ccc[nH]1] | structure 74 |
| 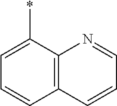 [SMILES: *c1cccc2cccnc12] | structure 75 |
| 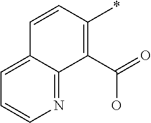 [SMILES: *c1ccc2cccnc2c1C(=O)O] | structure 76 |
| 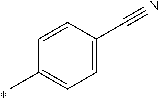 | structure 77 |
| 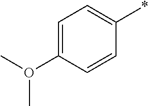 | structure 78 |
| 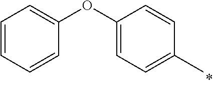 | structure 79 |
| 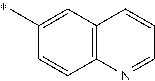 | structure 80 |
| 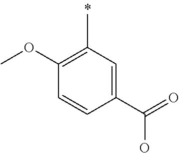 | structure 81 |
| 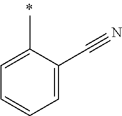 | structure 82 |
| 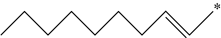 | structure 83 |
| 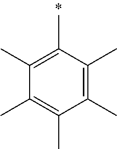 | structure 84 |
| 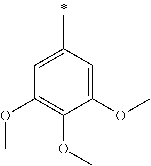 | structure 85 |
| 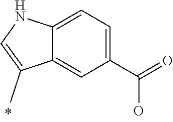 | structure 86 |
| 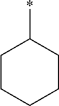 [SMILES: *C1CCCCC1] | structure 87 |
| 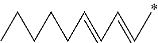 | structure 88 |
| 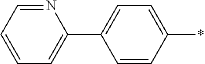 [SMILES: *c1ccc(-c2ccccn2)cc1] | structure 89 |
| 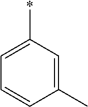 [SMILES: *c1cccc(C)c1] | structure 90 |
| 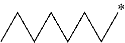 | structure 91 |
| 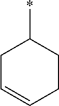 [SMILES: *C1CC=CCC1] | structure 92 |
| 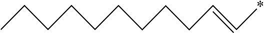 [SMILES: */C=C/CCCCCCCCC] | structure 93 |
| 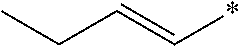 [SMILES: */C=C/CC] | structure 94 |
| 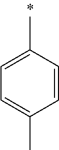 | structure 95 |
| 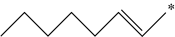 | structure 96 |
| 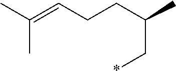 [SMILES: *C[C@H](C)CCC=C(C)C] | structure 97 |
| 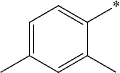 | structure 98 |
| 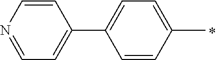 | structure 99 |
| 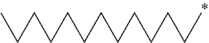 | structure 100 |
| 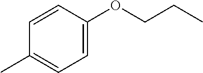 | structure 101 |
| 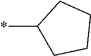 [SMILES: *C1CCCC1] | structure 102 |
| 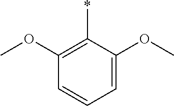 | structure 103 |
| 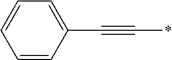 [SMILES: *C#Cc1ccccc1] | structure 104 |
| 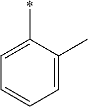 [SMILES: *c1ccccc1C] | structure 105 |
| 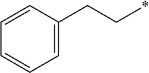 [SMILES: *CCc1ccccc1] | structure 106 |
| 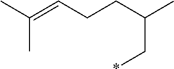 [SMILES: *CC(C)CCC=C(C)C] | structure 107 |
| 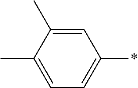 [SMILES: *c1ccc(C)c(C)c1] | structure 108 |
| 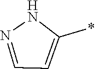 [SMILES: *c1ccn[nH]1] | structure 109 |
| 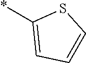 [SMILES: *c1cccs1] | structure 110 |
| 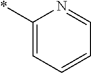 [SMILES: *c1ccccn1] | structure 111 |
| 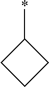 [SMILES: *C1CCC1] | structure 112 |
| 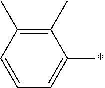 [SMILES: *c1cccc(C)c1C] | structure 113 |
| 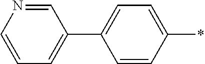 [SMILES: *c1ccc(-c2cccnc2)cc1] | structure 114 |
| 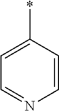 [SMILES: *c1ccncc1] | structure 115 |
| 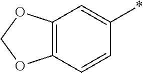 [SMILES: *c1ccc2c(c1)OCO2] | structure 116 |
| 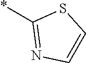 [SMILES: *c1nccs1] | structure 117 |
| 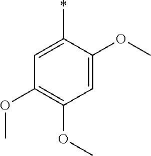 [SMILES: *c1cc(OC)c(OC)cc1OC] | structure 118 |
| 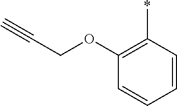 [SMILES: *c1ccccc1OCC#C] | structure 119 |
| 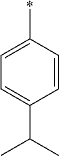 [SMILES: *c1ccc(C(C)C)cc1] | structure 120 |
| 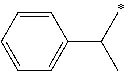 | structure 121 |
| 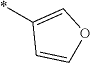 [SMILES: *c1ccoc1] | structure 122 |
| 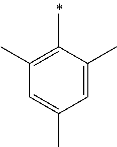 | structure 123 |
| 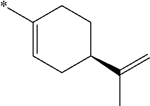 [SMILES: *C1=CC[C@H](C(=C)C)CC1] | structure 124 |
| 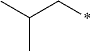 [SMILES: *CC(C)C] | structure 125 |
| 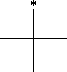 [SMILES: *C(C)(C)C] | structure 126 |
| 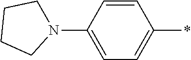 [SMILES: *c1ccc(N2CCCC2)cc1] | structure 127 |
| 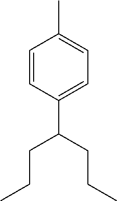 | structure 128 |
| 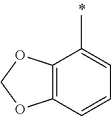 | structure 129 |
| 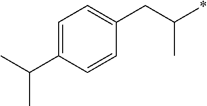 [SMILES: *C(C)Cc1ccc(C(C)C)cc1] | structure 130 |
| 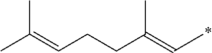 | structure 131 |
| 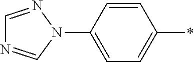 [SMILES: *c1ccc(-n2cncn2)cc1] | structure 132 |
| 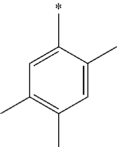 | structure 133 |
| 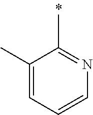 | structure 134 |
| 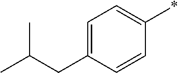 [SMILES: *c1ccc(CC(C)C)cc1] | structure 135 |
| 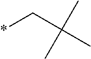 [SMILES: *CC(C)(C)C] | structure 136 |
| 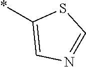 [SMILES: *c1cncs1] | structure 137 |
| 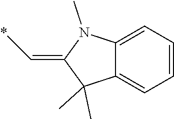 | structure 138 |
| 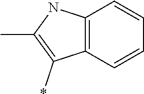 [SMILES: *c1c(C)[nH]c2ccccc12] | structure 139 |
| 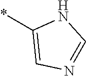 [SMILES: *c1cnc[nH]1] | structure 140 |

TABLE 2-3

| | |
|---|---|
| 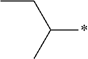 [SMILES: *C(C)CC] | structure 141 |
| 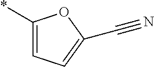 [SMILES: *c1ccc(C#N)o1] | structure 142 |
| 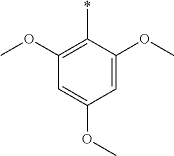 | structure 143 |
| 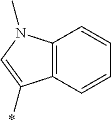 [SMILES: *c1cn(C)c2ccccc12] | structure 144 |
| 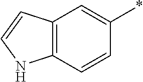 [SMILES: *c1ccc2[nH]ccc2c1] | structure 145 |
| 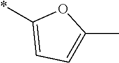 [SMILES: *c1ccc(C)o1] | structure 146 |
| 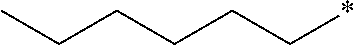 [SMILES: *CCCCCC] | structure 147 |
| 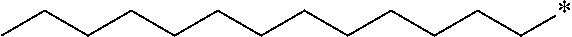 [SMILES: *CCCCCCCCCCCCC] | structure 148 |
| 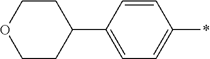 [SMILES: *c1ccc(C2CCOCC2)cc1] | structure 149 |
| 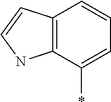 [SMILES: *c1cccc2cc[nH]c12] | structure 150 |
| 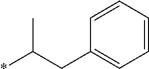 [SMILES: *C(C)Cc1ccccc1] | structure 151 |
| 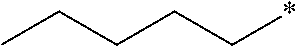 [SMILES: *CCCCC] | structure 152 |
| 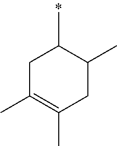 | structure 153 |
| 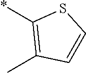 [SMILES: *c1sccc1C] | structure 154 |
| 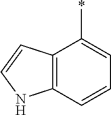 [SMILES: *c1cccc2[nH]ccc12] | structure 155 |
| 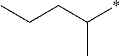 [SMILES: *C(C)CCC] | structure 156 |
| 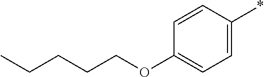 [SMILES: *c1ccc(OCCCCC)cc1] | structure 157 |
| 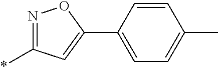 [SMILES: *c1cc(-c2ccc(C)cc2)on1] | structure 158 |
| 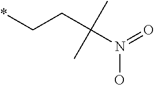 | structure 159 |
| 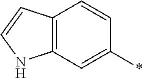 [SMILES: *c1ccc2cc[nH]c2c1] | structure 160 |
| 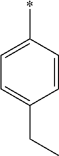 [SMILES: *c1ccc(CC)cc1] | structure 161 |
| 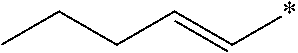 [SMILES: */C=C/CCC] | structure 162 |
| 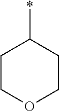 [SMILES: *C1CCOCC1] | structure 163 |
| 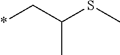 [SMILES: *CC(C)SC] | structure 164 |
| 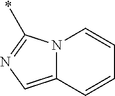 [SMILES: *c1ncc2ccccn12] | structure 165 |
| 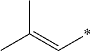 [SMILES: *C=C(C)C] | structure 166 |
| 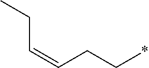 [SMILES: *CC/C=C\CC] | structure 167 |
| 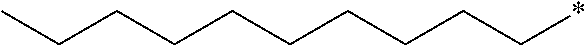 [SMILES: *CCCCCCCCCC] | structure 168 |
| 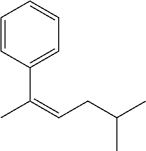 | structure 169 |
| 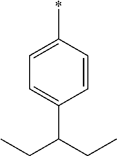 [SMILES: *c1ccc(C(CC)CC)cc1] | structure 170 |
| 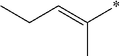 [SMILES: */C(C)=C/CC] | structure 171 |
| 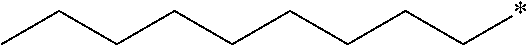 [SMILES: *CCCCCCCCC] | structure 172 |
| 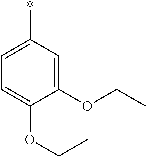 [SMILES: *c1ccc(OCC)c(OCC)c1] | structure 173 |
| 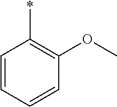 [SMILES: *c1ccccc1OC] | structure 174 |
| 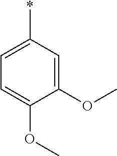 [SMILES: *c1ccc(OC)c(OC)c1] | structure 175 |
| 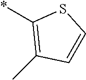 [SMILES: *c1sccc1C] | structure 176 |
| 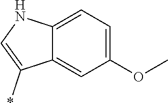 [SMILES: *c1c[nH]c2ccc(OC)cc12] | structure 177 |
| 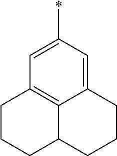 [SMILES: *c1cc2c3c(c1)CCCC3CCC2] | structure 178 |
| 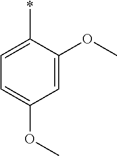 [SMILES: *c1ccc(OC)cc1OC] | structure 179 |
| 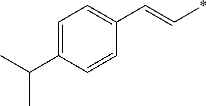 [SMILES: */C=C/c1ccc(C(C)C)cc1] | structure 180 |
| 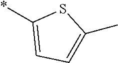 [SMILES: *c1ccc(C)s1] | structure 181 |
| 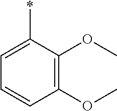 [SMILES: *c1cccc(OC)c1OC] | structure 182 |
| 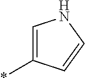 [SMILES: *c1cc[nH]c1] | structure 183 |
| 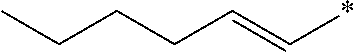 [SMILES: */C=C/CCCC] | structure 184 |
| 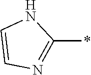 [SMILES: *c1ncc[nH]1] | structure 185 |
| 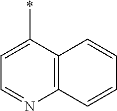 [SMILES: *c1ccnc2ccccc12] | structure 186 |
| 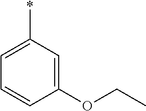 [SMILES: *c1cccc(OCC)c1] | structure 187 |
| 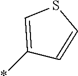 | structure 188 |
| 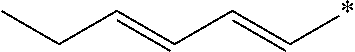 [SMILES: */C=C/C=C/CC] | structure 189 |
| 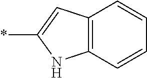 [SMILES: *c1cc2ccccc2[nH]1] | structure 190 |
| 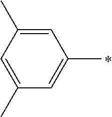 | structure 191 |
| 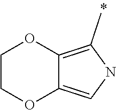 | structure 192 |
| 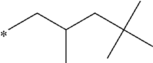 | structure 193 |
| 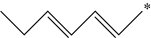 | structure 194 |
| 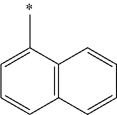 | structure 195 |
| 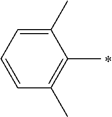 | structure 196 |
| 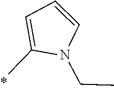 | structure 197 |
| 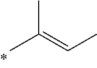 | structure 198 |
| 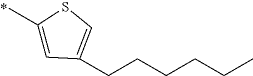 | structure 199 |
| 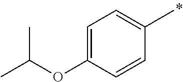 | structure 200 |
| 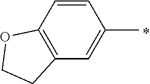 | structure 201 |
| 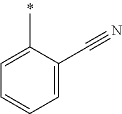 | structure 202 |
| 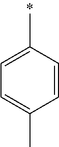 | structure 203 |
| 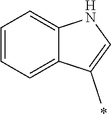 | structure 204 |
| 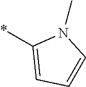 [SMILES: *c1cccn1C] | structure 205 |
| 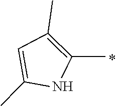 | structure 206 |
| 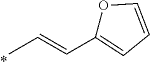 | structure 207 |
| 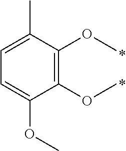 | structure 208 |
| 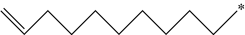 | structure 209 |
| 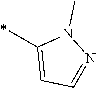 | structure 210 |

TABLE 2-4

| | |
|---|---|
| 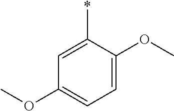 | structure 211 |
| 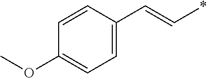 [SMILES: */C=C/c1ccc(OC)cc1] | structure 212 |
| 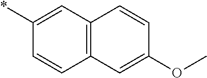 [SMILES: *c1ccc2cc(OC)ccc2c1] | structure 213 |
| 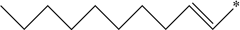 [SMILES: */C=C/CCCCCCCC] | structure 214 |
| 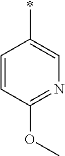 [SMILES: *c1ccc(OC)nc1] | structure 215 |
| 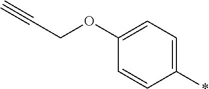 [SMILES: *c1ccc(OCC#C)cc1] | structure 216 |
| 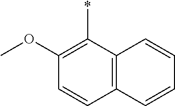 [SMILES: *c1c(OC)ccc2ccccc12] | structure 217 |
| 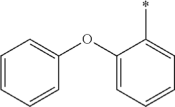 [SMILES: *c1ccccc1Oc1ccccc1] | structure 218 |
| 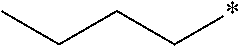 [SMILES: *CCCC] | structure 219 |
| 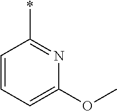 [SMILES: *c1cccc(OC)n1] | structure 220 |
| 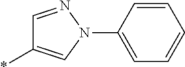 [SMILES: *c1cnn(-c2ccccc2)c1] | structure 221 |
| 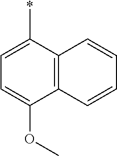 [SMILES: *c1ccc(OC)c2ccccc12] | structure 222 |
| 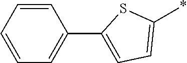 [SMILES: *c1ccc(-c2ccccc2)s1] | structure 223 |
| 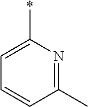 [SMILES: *c1cccc(C)n1] | structure 224 |
| 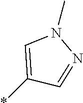 [SMILES: *c1cnn(C)c1] | structure 225 |
| 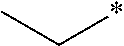 [SMILES: *CC] | structure 226 |
| 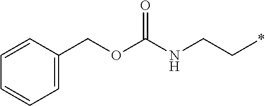 [SMILES: *CCNC(=O)OCc1ccccc1] | structure 227 |

R₄ in the formula (2), R₈ in the formula (4), and R₁₅ in the formula (6) each are most preferably selected from the structures shown in the Examples described hereinafter.

C in the formula (2) represents R₁₂—R₁₃—*, in which * represents a bonding position, R₁₂ represents a tertiary amino group having 2 to 6 carbon atoms, and R₁₃ represents an aliphatic compound having 2 to 30 carbon atoms, which may have a hydroxy group, and may have an ester bond.

C in the formula (2) preferably represents any of the structures represented by the following formulae (7), (8), and (9).

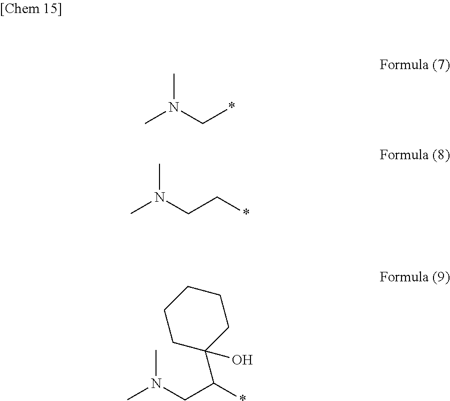

In the formulae (7) to (9), * represents a bonding position.

G in the formulae (3), (5), and (6) represents a hydrogen atom or an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur.

The group represented by G may be substituted at any one substitution position of four substitution positions of the aromatic ring shown in formulae (3), (5), and (6), or may be substituted at two or more substitution positions thereof, and in the case where the groups represented by G are substituted at two or more substitution positions, the multiple groups may be the same as or different from each other, and the groups at the adjacent two substitution positions may form a ring.

The four substitution positions are as follows.

In the formula (3), they mean the four substitution positions of the aromatic ring other than the two substitution positions, i.e., the position substituted with the group having R₁ and the position substituted with the group having R₅, R₆, and R₇.

In the formula (5), they mean the four substitution positions of the aromatic ring other than the two substitution positions, i.e., the position substituted with the group having R₁ and the position substituted with the group having R₉, R₁₀, and R₁₆.

In the formula (6), they mean the four substitution positions of the aromatic ring other than the two substitution positions, i.e., the position substituted with the group having R₁ and the position substituted with the group having R₉, R₁₁, and R₁₅.

G in the formulae (3), (5), and (6) preferably represents a hydrogen atom or an aliphatic compound or an aromatic compound having 1 to 15 carbon atoms, 0 to 2 oxygen atoms, 0 to 2 nitrogen atoms, and 0 to 1 sulfur atom, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur. The atoms or groups represented by G are preferably independent at each of the four substitution positions.

The group represented by G is more preferably selected from the structure groups shown in Table 3-1. In Table 3-1, * represents a bonding point, and in the case where multiple bonding points exist, the * represents the bonding point to the adjacent substitution position on the benzene ring shown in the formulae (3), (5), and (6).

TABLE 3-1

| | |
|---|---|
| 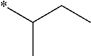 [SMILES: *C(C)CC] | structure 1 |
| 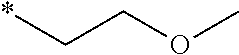 [SMILES: *CCOC] | structure 2 |
| 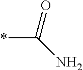 [SMILES: *C(N)=O] | structure 3 |
| 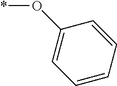 [SMILES: *Oc1ccccc1] | structure 4 |
| 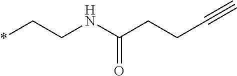 [SMILES: *CCNC(=O)CCC#C] | structure 5 |
| 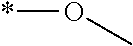 [SMILES: *OC] | structure 6 |
| 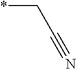 [SMILES: *CC#N] | structure 7 |
| 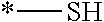 [SMILES: *S] | structure 8 |
| 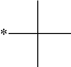 [SMILES: *C(C)(C)C] | structure 9 |
| 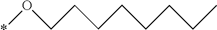 [SMILES: *OCCCCCCCC] | structure 10 |
| 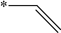 [SMILES: *C=C] | structure 11 |
| 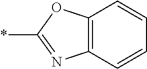 [SMILES: *c1nc2ccccc2o1] | structure 12 |
| 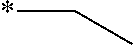 [SMILES: *CC] | structure 13 |
| 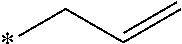 [SMILES: *CC=C] | structure 14 |
| 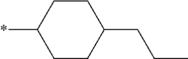 [SMILES: *C1CCC(CCC)CC1] | structure 15 |
| 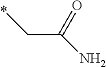 [SMILES: *CC(N)=O] | structure 16 |
| 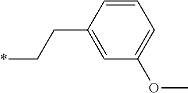 [SMILES: *CCc1cccc(OC)c1] | structure 17 |
| 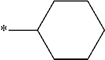 [SMILES: *C1CCCCC1] | structure 18 |
| 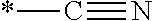 [SMILES: *C#N] | structure 19 |
| 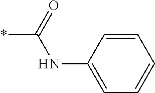 [SMILES: *C(=O)Nc1ccccc1] | structure 20 |
| 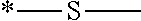 [SMILES: *SC] | structure 21 |
| 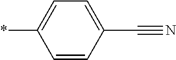 [SMILES: *c1ccc(C#N)cc1] | structure 22 |
| 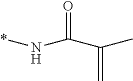 [SMILES: *NC(=O)C(=C)C] | structure 23 |
| 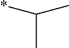 [SMILES: *C(C)C] | structure 24 |
| 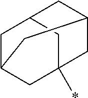 [SMILES: *C12CC3CC(CC(C3)C1)C2] | structure 25 |
| 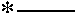 [SMILES: *C] | structure 26 |
| 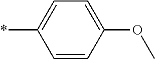 [SMILES: *c1ccc(OC)cc1] | structure 27 |
| 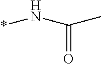 [SMILES: *NC(C)=O] | structure 28 |
| 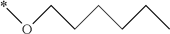 [SMILES: *OCCCCCC] | structure 29 |
| 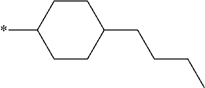 [SMILES: *C1CCC(CCCC)CC1] | structure 30 |
| 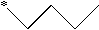 [SMILES: *CCCC] | structure 31 |
| 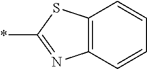 [SMILES: *c1nc2ccccc2s1] | structure 32 |
| 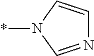 [SMILES: *n1ccnc1] | structure 33 |
| 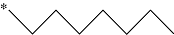 | structure 34 |
| 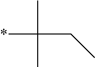 [SMILES: *C(C)(C)CC] | structure 35 |
| 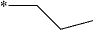 [SMILES: *CCC] | structure 36 |
| 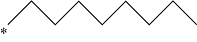 | structure 37 |
| 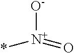 [SMILES: *[N+](=O)[O-]] | structure 38 |
| 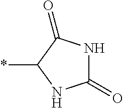 [SMILES: *C1NC(=O)NC1=O] | structure 39 |
| 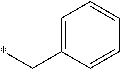 [SMILES: *Cc1ccccc1] | structure 40 |
| 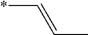 [SMILES: */C=C/C] | structure 41 |
| 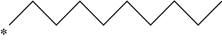 | structure 42 |
| 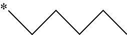 | structure 43 |
| 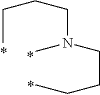 | structure 44 |
| 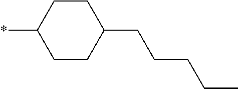 [SMILES: *C1CCC(CCCCC)CC1] | structure 45 |
| 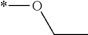 [SMILES: *OCC] | structure 46 |
| 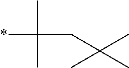 [SMILES: *C(C)(C)CC(C)(C)C] | structure 47 |
| 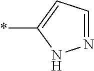 [SMILES: *c1ccn[nH]1] | structure 48 |
| 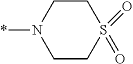 [SMILES: *N1CCS(=O)(=O)CC1] | structure 49 |
| 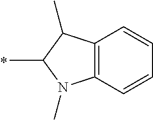 | structure 50 |
| 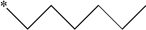 [SMILES: *CCCCCC] | structure 51 |
| 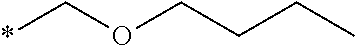 [SMILES: *COCCCC] | structure 52 |
| 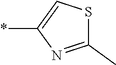 [SMILES: *c1csc(C)n1] | structure 53 |
| 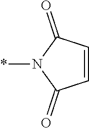 [SMILES: *N1C(=O)C=CC1=O] | structure 54 |
| 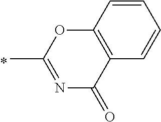 | structure 55 |
| 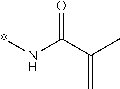 [SMILES: *NC(=O)C(=C)C] | structure 56 |
| 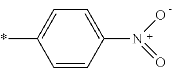 | structure 57 |
| 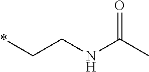 [SMILES: *CCNC(C)=O] | structure 58 |
| 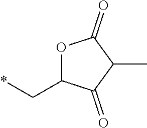 [SMILES: *CC1OC(=O)C(C)C1=O] | structure 59 |
| 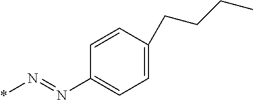 [SMILES: */N=N/c1ccc(CCCC)cc1] | structure 60 |
| 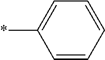 [SMILES: *c1ccccc1] | structure 61 |
| 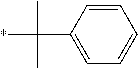 [SMILES: *C(C)(C)c1ccccc1] | structure 62 |
| 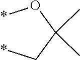 [SMILES: *CC(C)(C)O*] | structure 63 |
| 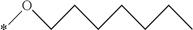 [SMILES: *OCCCCCCC] | structure 64 |
| 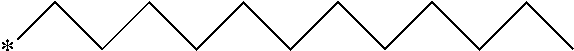 [SMILES: *CCCCCCCCCCCC] | structure 65 |
| 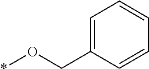 [SMILES: *OCc1ccccc1] | structure 66 |
| 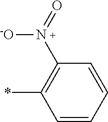 | structure 67 |
| 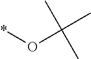 [SMILES: *OC(C)(C)C] | structure 68 |
| 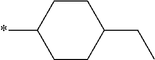 [SMILES: *C1CCC(CC)CC1] | structure 69 |
| 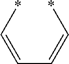 [SMILES: */C=C\C=C/*] | structure 70 |

G in the formulae (3), (5), and (6) is most preferably selected from the structures shown in the Examples described hereinafter.

R₁ in the formulae (3), (4), (5), and (6) preferably represents an alkyl group having 10 to 30 carbon atoms, an alkenyl group having 10 to 30 carbon atoms, or an alkynyl group having 10 to 30 carbon atoms, more preferably an alkyl group having 10 to 30 carbon atoms or an alkenyl group having 10 to 30 carbon atoms, and further preferably an alkyl group having 10 to 20 carbon atoms or an alkenyl group having 10 to 20 carbon atoms.

R₅ and R₆ in the formulae (3) and (4) each independently represent an aliphatic compound having 1 to 30 carbon atoms, 0 to 5 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, and a heterocyclic ring having at least any atom selected from nitrogen, oxygen, and sulfur, and R₅ and R₆ each may be independent from each other, or may be connected to each other to form a ring.

R₅ and R₆ each preferably independently represent an aliphatic compound having 1 to 15 carbon atoms, 0 to 2 oxygen atoms, 0 to 1 nitrogen atom, and 0 to 1 sulfur atom, which may form a ring, may contain an unsaturated bond, and may have at least any structure selected from the group consisting of an ether group, an ester group, a nitrile group, an amide group, a nitro group, a sulfide group, a sulfonamide group, and a heterocyclic ring having at least any atom selected from nitrogen, oxygen, and sulfur, and R₅ and R₆ each may be independent from each other, or may be connected to each other to form a ring.

The groups represented by R₅ and R₆ are more preferably selected from the structures shown in Tables 4-1 to 4-3. In the Tables 4-1 to 4-3, * represents a bonding point, and two bonding points show the bonding point to nitrogen in the formulae (3) and (4).

TABLE 4-1

| | |
|---|---|
| 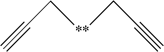 | structure 1 |
| 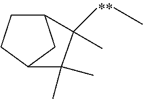 | structure 2 |
| 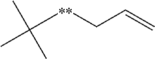 | structure 3 |
| 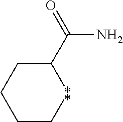 | structure 4 |
| 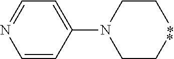 | structure 5 |
| 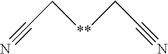 | structure 6 |
| 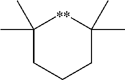 | structure 7 |
| 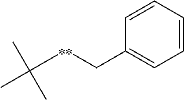 | structure 8 |
| 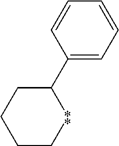 | structure 9 |
| 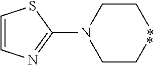 | structure 10 |
| 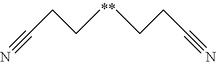 | structure 11 |
| 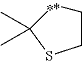 | structure 12 |
| 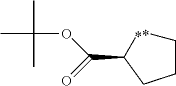 | structure 13 |
| 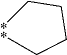 | structure 14 |
| 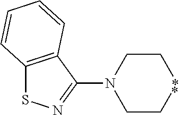 [SMILES: CCCN(CCC)c1nsc2ccccc12] | structure 15 |
| 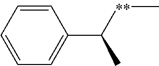 | structure 16 |
| 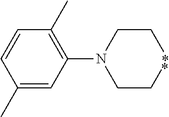 | structure 17 |
| 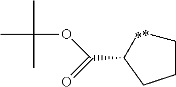 | structure 18 |
| 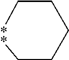 | structure 19 |
| 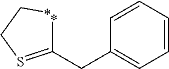 | structure 20 |
| 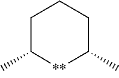 | structure 21 |
| 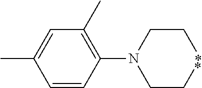 | structure 22 |
| 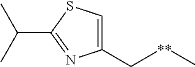 | structure 23 |
| 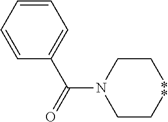 | structure 24 |
| 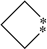 | structure 25 |
| 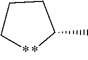 | structure 26 |
| 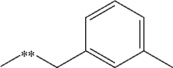 | structure 27 |
| 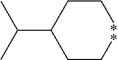 | structure 28 |
| 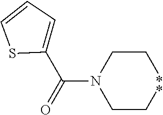 | structure 29 |
| 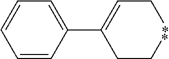 | structure 30 |
| 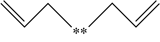 | structure 31 |
| 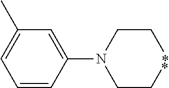 | structure 32 |
| 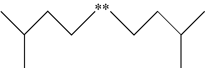 | structure 33 |
| 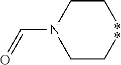 | structure 34 |
| 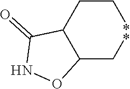 | structure 35 |
| 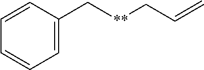 | structure 36 |
| 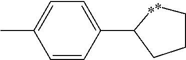 | structure 37 |
| 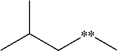 | structure 38 |
| 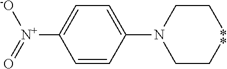 | structure 39 |
| 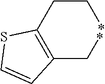 | structure 40 |
| 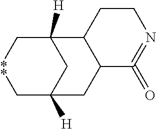 | structure 41 |
| 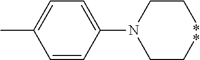 | structure 42 |
| 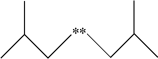 | structure 43 |
| 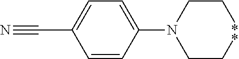 | structure 44 |
| 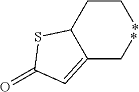 | structure 45 |
| 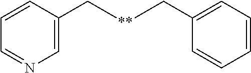 | structure 46 |
| 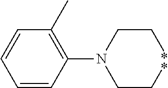 | structure 47 |
| 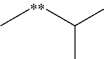 | structure 48 |
| 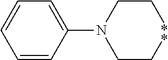 | structure 49 |
| 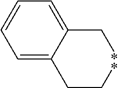 | structure 50 |
| 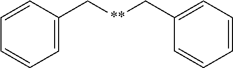 | structure 51 |
| 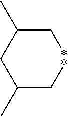 | structure 52 |
| 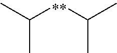 | structure 53 |
| 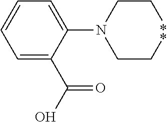 [SMILES: CCCN(CCC)c1ccccc1C(=O)O] | structure 54 |
| 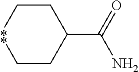 | structure 55 |
| 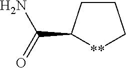 | structure 56 |
| 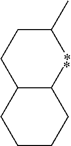 | structure 57 |
| 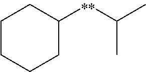 | structure 58 |
| 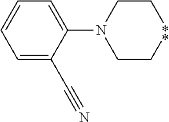 | structure 59 |
| 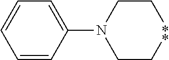 | structure 60 |
| 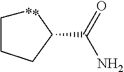 | structure 61 |
| 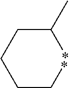 | structure 62 |
| 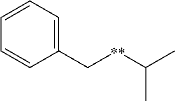 | structure 63 |
| 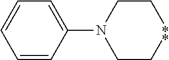 | structure 64 |
| 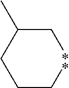 | structure 65 |
| 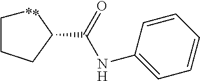 | structure 66 |
| 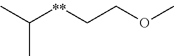 | structure 67 |
| 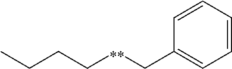 | structure 68 |
| 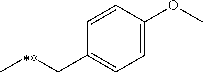 | structure 69 |
| 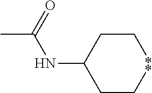 | structure 70 |

TABLE 4-2

| | |
|---|---|
| 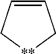 | structure 71 |
| 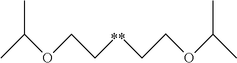 | structure 72 |
| 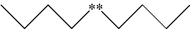 | structure 73 |
| 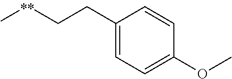 | structure 74 |
| 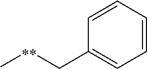 | structure 75 |
| 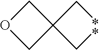 | structure 76 |
| 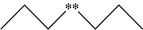 | structure 77 |
| 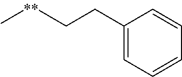 | structure 78 |
| 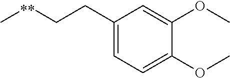 | structure 79 |
| 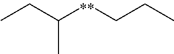 | structure 80 |
| 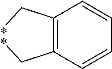 | structure 81 |
| 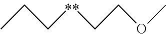 | structure 82 |
| 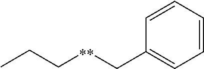 | structure 83 |
| 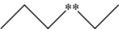 | structure 84 |
| 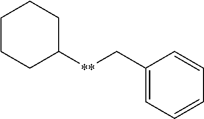 | structure 85 |
| 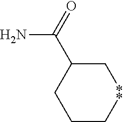 | structure 86 |
| 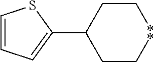 | structure 87 |
| 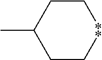 | structure 88 |
| 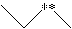 | structure 89 |
| 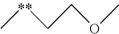 | structure 90 |
| 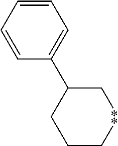 | structure 91 |
| 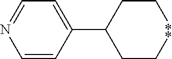 | structure 92 |
| 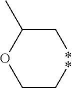 | structure 93 |
| 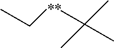 | structure 94 |
| 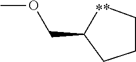 | structure 95 |
| 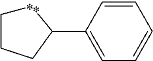 | structure 96 |
| 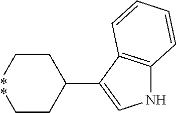 [SMILES: CCCC(CCC)c1c[nH]c2ccccc12] | structure 97 |
| 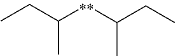 | structure 98 |
| 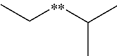 | structure 99 |
| 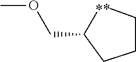 | structure 100 |
| 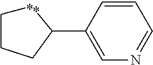 | structure 101 |
| 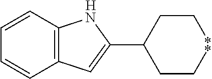 | structure 102 |
| 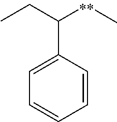 | structure 103 |
| 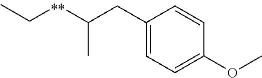 | structure 104 |
| 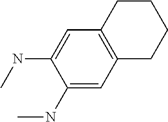 | structure 105 |
| 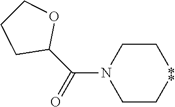 | structure 106 |
| 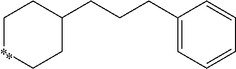 [SMILES: CCCC(CCC)CCCc1ccccc1] | structure 107 |
| 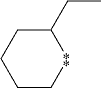 | structure 108 |
| 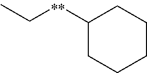 | structure 109 |
| 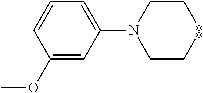 | structure 110 |
| 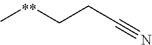 | structure 111 |
| 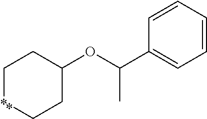 [SMILES: CCCC(CCC)OC(C)c1ccccc1] | structure 112 |
| 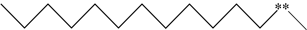 | structure 113 |
| 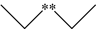 | structure 114 |
| 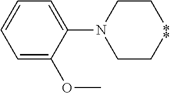 | structure 115 |
| 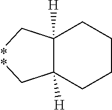 | structure 116 |
| 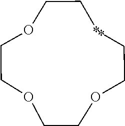 | structure 117 |
| 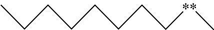 | structure 118 |
| 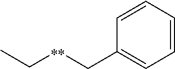 | structure 119 |
| 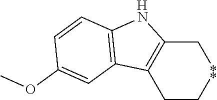 | structure 120 |
| 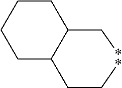 [SMILES: *CCC1CCCCC1C*] | structure 121 |
| 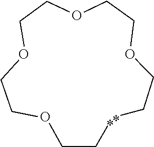 [SMILES: CCCOCCOCCOCCOCCC] | structure 122 |
| 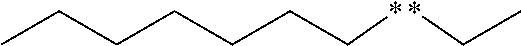 | structure 123 |
| 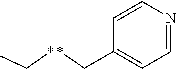 | structure 124 |
| 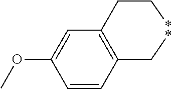 [SMILES: *CCc1cc(OC)ccc1C*] | structure 125 |
| 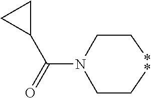 [SMILES: *CCN(CC*)C(=O)C1CC1] | structure 126 |
| 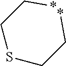 [SMILES: *CCSCC*] | structure 127 |
| 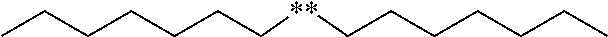 | structure 128 |
| 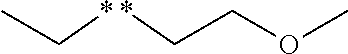 | structure 129 |
| 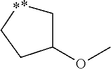 [SMILES: *CCC(C*)OC] | structure 130 |
| 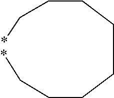 [SMILES: *CCCCCCCC*] | structure 131 |
| 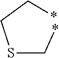 [SMILES: *CCSC*] | structure 132 |
| 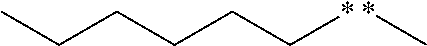 | structure 133 |
| 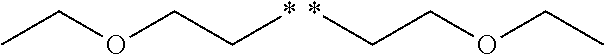 | structure 134 |
| 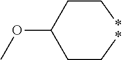 [SMILES: *CCC(CC*)OC] | structure 135 |
| 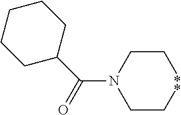 | structure 136 |
| 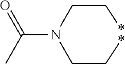 [SMILES: *CCN(CC*)C(C)=O] | structure 137 |
| 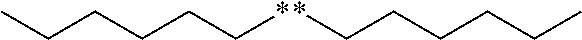 | structure 138 |
| 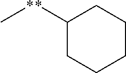 | structure 139 |
| 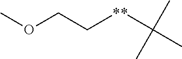 | structure 140 |

TABLE 4-3

| | |
|---|---|
| 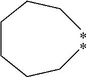 | structure 141 |
| 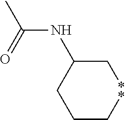 | structure 142 |
| 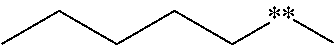 | structure 143 |
| 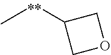 | structure 144 |
| 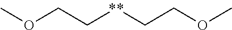 | structure 145 |
| 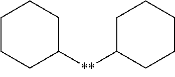 | structure 146 |
| 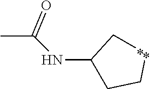 | structure 147 |
| 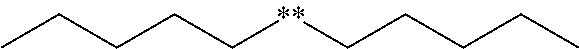 | structure 148 |
| 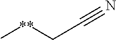 | structure 149 |
| 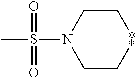 | structure 150 |
| 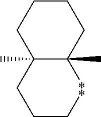 | structure 151 |
| 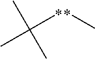 | structure 152 |
| 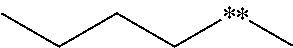 | structure 153 |
| 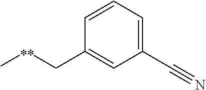 | structure 154 |
| 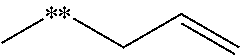 | structure 155 |
| 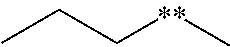 | structure 156 |
| 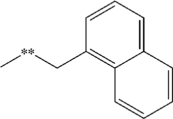 | structure 157 |
| 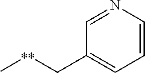 | structure 158 |
| 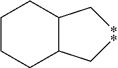 | structure 159 |
| 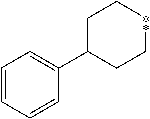 | structure 160 |
| 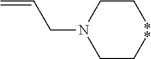 | structure 161 |
| 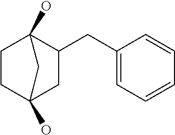 [SMILES: O[C@@]12CC[C@@](O)(C1)C(Cc1ccccc1)C2] | structure 162 |
| 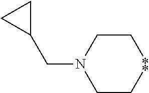 | structure 163 |
| 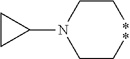 | structure 164 |
| 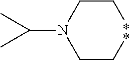 | structure 165 |
| 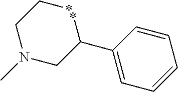 | structure 166 |
| 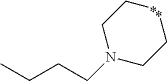 | structure 167 |
| 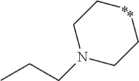 | structure 168 |
| 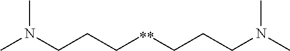 | structure 169 |
| 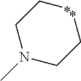 | structure 170 |
| 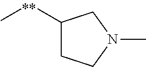 | structure 171 |
| 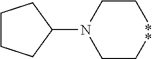 | structure 172 |
| 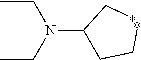 | structure 173 |
| 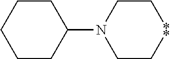 | structure 174 |
| 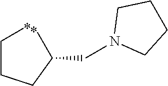 | structure 175 |
| 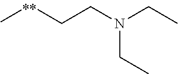 | structure 176 |
| 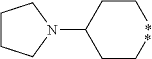 | structure 177 |
| 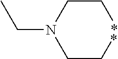 | structure 178 |
| 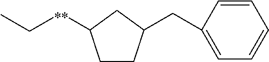 | structure 179 |
| 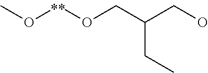 | structure 180 |
| 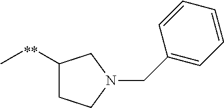 | structure 181 |
| 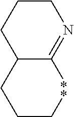 | structure 182 |
| 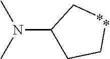 | structure 183 |
| 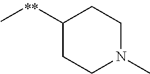 | structure 184 |
| 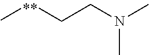 | structure 185 |
| 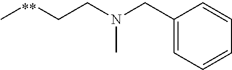 | structure 186 |
| 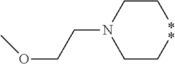 | structure 187 |
| 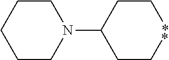 | structure 188 |

R₅ and R₆ each are more preferably selected from the structures shown in the Examples described hereinafter.

R₇ in the formula (3) represents an aliphatic compound having 2 to 30 carbon atoms, 2 to 10 oxygen atoms, 0 to 5 nitrogen atoms, and 0 to 2 sulfur atoms, having an ester bond, which may have at least any structure selected from the group consisting of a hydroxy group, an ether group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound.

R₇ preferably represents an aliphatic compound having 5 to 30 carbon atoms, 2 to 4 oxygen atoms, 0 to 1 nitrogen atom, and 0 to 1 sulfur atom, having an ester bond, which may contain an unsaturated bond, may have a branch, may form a ring, and may have at least any structure selected from the group consisting of an aromatic ring, an ether group, and a heterocyclic ring having at least any atom selected from nitrogen, oxygen, and sulfur.

R₇ more preferably contains the structures shown in Table 5, and may have a structure obtained by replacing the hydroxy group in Table 5 by an ester containing an aliphatic hydrocarbon having 10 to 30 carbon atoms. In Table 5, * represents a bonding point.

TABLE 5

| | |
|---|---|
| 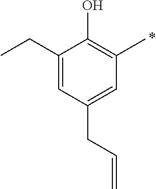 | structure 1 |
| 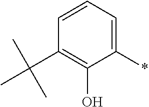 [SMILES: *c1cccc(C(C)(C)C)c1O] | structure 2 |
| 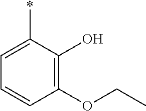 [SMILES: *c1cccc(OCC)c1O] | structure 3 |
| 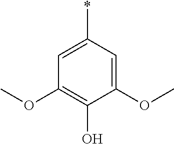 | structure 4 |
| 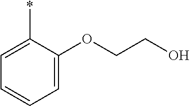 [SMILES: *c1ccccc1OCCO] | structure 5 |
| 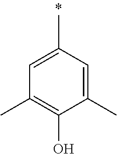 | structure 6 |
| 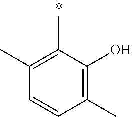 | structure 7 |
| 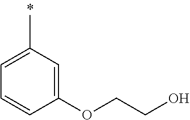 | structure 8 |
| 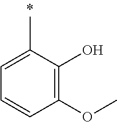 | structure 9 |
| 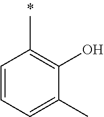 | structure 10 |
| 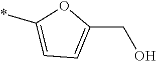 [SMILES: *c1ccc(CO)o1] | structure 11 |
| 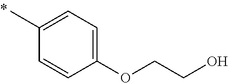 | structure 12 |
| 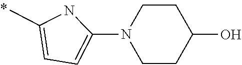 | structure 13 |
| 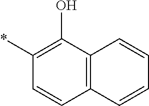 [SMILES: *c1ccc2ccccc2c1O] | structure 14 |

E in the formula (4) represents a hydrogen atom, a nitro group, a methoxy group, a nitrile group, a methyl group, a tert-butyl group, or a 1,1-dimethylpropyl group, and preferably a hydrogen atom.

F in the formula (4) represents R₁′—CO—O—R₁₄—*, in which * represents a bonding position, and R₁₄ represents an aliphatic hydrocarbon group having 0 to 8 carbon atoms. R₁₄ preferably represents an alkylene group having 0 to 8 carbon atoms, an alkenediyl group having 0 to 8 carbon atoms, or an alkynediyl group having 0 to 8 carbon atoms, more preferably an alkylene group having 0 to 8 carbon atoms or an alkenediyl group having 0 to 8 carbon atoms, and further preferably an alkylene group having 0 to 8 carbon atoms.

R₁′ has the same meaning as in the formula (1). R₁′ preferably represents an alkyl group having 10 to 30 carbon atoms, an alkenyl group having 10 to 30 carbon atoms, or an alkynyl group having 10 to 30 carbon atoms, and more preferably an alkyl group having 10 to 20 carbon atoms or an alkenyl group having 10 to 20 carbon atoms.

R₉ in the formulae (5) and (6) represents an aliphatic hydrocarbon group having 10 to 30 carbon atoms, preferably an alkyl group having 10 to 30 carbon atoms, an alkenyl group having 10 to 30 carbon atoms, or an alkynyl group having 10 to 30 carbon atoms, more preferably an alkyl group having 10 to 30 carbon atoms or an alkenyl group having 10 to 30 carbon atoms, and further preferably an alkyl group having 10 to 20 carbon atoms or an alkenyl group having 10 to 20 carbon atoms.

R₁₀ in the formula (5) represents an aliphatic compound having at least one linear or cyclic tertiary amine structure, having 2 to 15 carbon atoms, 0 to 10 oxygen atoms, and 1 to 5 nitrogen atoms, which may have at least any functional group selected from the group consisting of an ester group, an amide group, a carbamate group, an ether group, a urea group, a thioamide group, a sulfide group, and a sulfonamide group, and may be bonded to an aromatic compound. R₁₀ preferably represents any of structures represented by the following formulae (10) to (14).

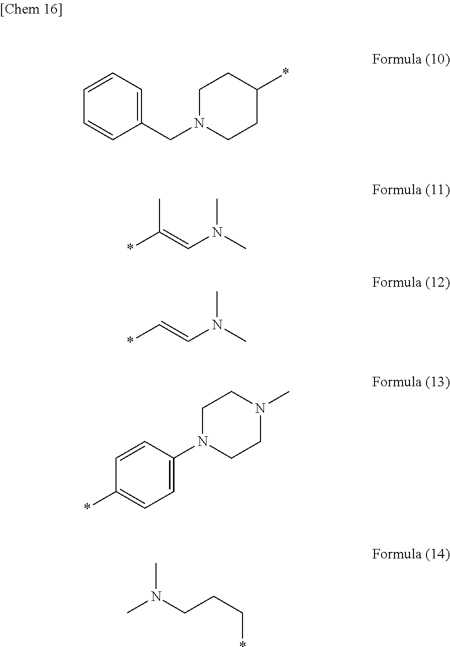

R₁₁ in the formula (6) represents an aliphatic compound having at least one linear or cyclic tertiary amine structure, having 1 to 15 carbon atoms, which may be bonded to an aromatic compound. R₁₁ is preferably selected from the structures shown in Table 6-1. In Table 6-1, * represents a bonding point.

TABLE 6-1

| | |
|---|---|
| 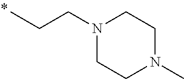 | structure 1 |
| 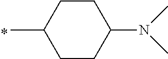 | structure 2 |
| 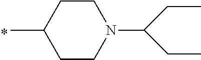 | structure 3 |
| 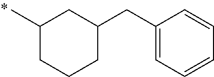 | structure 4 |
| 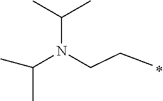 | structure 5 |
| 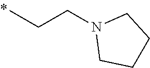 | structure 6 |
| 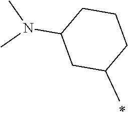 | structure 7 |
| 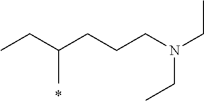 | structure 8 |
| 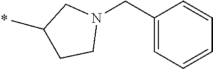 | structure 9 |
| 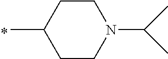 | structure 10 |
| 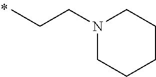 | structure 11 |
| 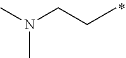 | structure 12 |
| 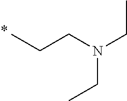 | structure 13 |
| 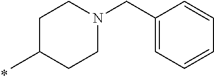 | structure 14 |
| 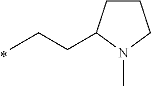 | structure 15 |
| 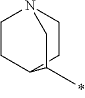 | structure 16 |
| 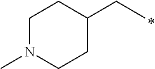 | structure 17 |
| 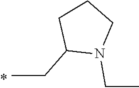 | structure 18 |
| 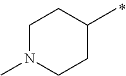 | structure 19 |
| 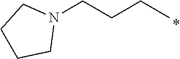 | structure 20 |
| 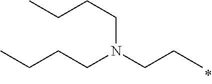 | structure 21 |
| 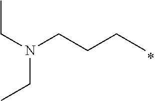 | structure 22 |
| 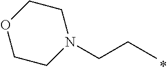 | structure 23 |
| 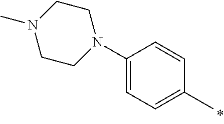 | structure 24 |

R₁₁ more preferably selected from the structures shown in the Examples.

The present invention also provides a lipid membrane structure containing the ionizable lipid described above as a constitutional lipid of the membrane. The lipid membrane structure of the present invention may further contains a nucleic acid.

In the description herein, the “lipid membrane structure” means a particle having such a membrane structure that the hydrophilic groups of the amphiphilic lipid are aligned toward the aqueous phase side of the interface. The “amphiphilic lipid” means a lipid that has both a hydrophilic group showing hydrophilicity and a hydrophobic group showing hydrophobicity. Examples of the amphiphilic lipid include an ionizable cationic lipid and a phospholipid.

The form of the lipid membrane structure of the present invention is not particularly limited, and examples of the form thereof in which the ionizable lipid of the present invention is dispersed in an aqueous solvent include a liposome (for example, a unilamellar liposome and a multilamellar liposome), an O/W type emulsion, a W/O type emulsion, a spherical micelle, a worm-like micelle, lipid nanoparticles (which may be abbreviated as “LNP” in the description herein), and a irregular lamellar structure. The lipid membrane structure of the present invention is preferably a liposome. In another embodiment of the present invention, the lipid membrane structure of the present invention is preferably LNP.

The lipid membrane structure of the present invention may further contain an additional constitutional component in addition to the ionizable lipid of the present invention. Examples of the additional constitutional component include a lipid (for example, a phospholipid (such as phorphatidylinositol, phosphatidylethanolamine, phosphatidylserine, phosphatidic acid, phosphatidylglycerol, and phosphatidylcholine), a glycolipid, a peptide lipid, cholesterol, a ionizable cationic lipid other than the ionizable lipid of the present invention, and a PEG lipid), a surfactant (for example, 3-[(3-cholamidopropyl)dimethylammonio]propanesulfonate, sodium cholate, octyl glycoside, and an N-D-gluco-N-methylalkanamide compound), polyethylene glycol, and a protein. The content of the additional constitutional component in the lipid membrane structure of the present invention is generally 5 to 95% by mol, preferably 10 to 90% by mol, and more preferably 30 to 80% by mol.

The content of the ionizable lipid of the present invention contained in the lipid membrane structure of the present invention is not particularly limited, and in the case where the lipid membrane structure is used as a nucleic acid transfer agent described hereinafter, in general, the ionizable lipid is contained in such an amount that is sufficient for transferring the nucleic acid. The content thereof is, for example, 5 to 100% by mol, preferably 10 to 90% by mol, and more preferably 20 to 70% by mol, based on the total lipids.

The lipid membrane structure of the present invention can be prepared in such a manner that the ionizable lipid and the other constitutional components (such as a lipid) are dispersed in a suitable solvent or dispersion medium, such as an aqueous solvent or an alcoholic solvent, which is then subjected to an operation for inducing organization depending on necessity.

Examples of the “operation for inducing organization” include methods known per se, such as an ethanol dilution method using a microfluidic channel or vortex mixing, a simple hydration method, an ultrasonic treatment, heating, vortex mixing, an ether injection method, a French press method, a cholate method, a Ca²⁺ fusion method, a freeze-thaw method, and a reverse-phase evaporation method, but are not limited thereto.

A nucleic acid can be transferred into cells in vivo and/or in vitro by bringing a lipid membrane structure containing the nucleic acid into contact with the cells. Therefore, the present invention provides a nucleic acid transfer agent containing the ionizable lipid of the present invention. The nucleic acid transfer agent of the present invention is preferably the lipid membrane structure of the present invention. The nucleic acid transfer agent of the present invention preferably contains a nucleic acid.

The nucleic acid transfer agent of the present invention can transfer any nucleic acid into cells. Examples of the type of nucleic acid include DNA, RNA, RNA chimeric nucleic acid, and DNA/RNA hybrid, but are not limited thereto. The nucleic acid may be any of single to triple strand, and is preferably single strand or double strand. The nucleic acid may be other types of a nucleotide that is an N-glycoside of purine or a pyrimidine base, or an oligomer having a non-nucleotide backbone (for example, a commercially available peptide nucleic acid (PNA)), or an oligomer having a special linkage (provided that the oligomer contains nucleotides arranged to permit base pairing and base attachment, as found in DNA or RNA), and the like. Furthermore, the nucleic acid may be, for example, a nucleic acid to which a known modification has been added, a labeled nucleic acid known in this field of art, a capped nucleic acid, a methylated nucleic acid, a nucleic acid in which one or more natural nucleotides are substituted with an analog, a nucleic acid having an intramolecular nucleotide modification, a nucleic acid having a non-charged linkage (for example, methylsulfonate, phosphotriester, phosphoramidate, and carbamate), a nucleic acid having a charged linkage or a sulfur-containing linkage (for example, phosphorothioate and phosphorodithioate), a nucleic acid having a side chain group, such as proteins (for example, a nuclease, a nuclease inhibitor, a toxin, an antibody, a signal peptide, and a poly-L-lysine) or a sugar (for example, a monosaccharide), a nucleic acid having an intercalating compound (for example, acridine and psoralen), a nucleic acid containing a chelating compound (for example, a metal, a radioactive metal, boron, and an oxidizing metal), a nucleic acid containing an alkylating agent, a nucleic acid having a modified linkage (for example, an α-anomeric nucleic acid), and the like.

The type of DNA that can be used in the present invention is not particularly limited, and can be appropriately selected depending on the intended purpose of use. Examples thereof include plasmid DNA, cDNA, antisense DNA, chromosomal DNA, PAC, BAC, and CpG oligos, in which plasmid DNA, cDNA, and antisense DNA are preferred, and plasmid DNA is more preferred. Circular DNA such as plasmid DNA can also be used as linear DNA through appropriate digestion with a restriction enzyme or the like.

The type of RNA that can be used in the present invention is not particularly limited, and can be appropriately selected depending on the intended purpose of use. Examples thereof include siRNA, miRNA, shRNA, antisense RNA, messenger RNA (mRNA), a single stranded RNA genome, a double stranded RNA genome, an RNA replicon, transfer RNA, and ribosomal RNA, in which siRNA, miRNA, shRNA, mRNA, antisense RNA, and an RNA replicon are preferred.

The nucleic acid used in the present invention is preferably purified by a method that is commonly used by those skilled in the art.

The nucleic acid transfer agent of the present invention containing a nucleic acid can be administered in vivo, for example, for the purpose of preventing and/or treating a disease. Accordingly, the nucleic acid used in the present invention preferably has a prophylactic and/or therapeutic activity against a prescribed disease (i.e., a prophylactic/therapeutic nucleic acid). Examples of the nucleic acid include a nucleic acid used in the so-called gene therapy.

For transferring a nucleic acid into cells using the nucleic acid transfer agent of the present invention, at the time of forming the lipid membrane structure of the present invention, the target nucleic acid is allowed to coexist to form a lipid membrane structure of the present invention containing the nucleic acid (i.e., the nucleic acid transfer agent of the present invention containing the nucleic acid). For example, in the case where a liposome as a lipid membrane structure is formed by the ethanol dilution method, an aqueous solution of the nucleic acid and an ethanol solution of the constitutional components of the lipid membrane structure of the present invention (such as the lipid) are vigorously mixed with a microfluidic channel or vortex mixing, and then the mixture is diluted with an appropriate buffer solution. In the case where a liposome as a lipid membrane structure is formed by the simple hydration method, the constitutional components of the lipid membrane structure of the present invention (such as the lipid) are dissolved in an appropriate organic solvent, the solution is placed in a glass container, and the solvent is removed by drying under reduced pressure to provide a lipid thin film. An aqueous solution of the nucleic acid is added thereto, and the film is hydrated, and then subjected to ultrasonication with Sonicator.

One embodiment of the lipid membrane structure of the present invention containing a nucleic acid is an LNP having the nucleic acid encapsulated by forming an electrostatic complex between the nucleic acid and an ionizable lipid. The LNP can be used as a drug delivery system for selectively delivering a nucleic acid and the like to specific cells, and for example, is useful as a DNA vaccine and a gene therapeutic agent for tumors through transfer of an antigen gene into dendritic cells, and a nucleic acid pharmaceutical that suppresses the expression of a target gene using RNA interference.

The particle diameter of the lipid membrane structure of the present invention containing a nucleic acid is not particularly limited, and is preferably 10 nm to 500 nm, and more preferably 30 nm to 300 nm. The particle diameter can be measured by using, for example, a particle size distribution measuring device, such as Zetasizer Nano (available from Malvern Panalytical Ltd.). The particle diameter of the lipid membrane structure can be appropriately regulated by the method of preparing the lipid membrane structure.

The surface potential (zeta potential) of the lipid membrane structure of the present invention containing a nucleic acid is not particularly limited, and is preferably −15 to +15 mV, and more preferably −10 to +10 mV. In the ordinary gene transfer, particles having a positively charged surface potential have mainly been used. While the use thereof is useful as a method for promoting the electrostatic interaction of heparan sulfate on the cell surface having a negative charge, and thereby promoting uptake into the cells, the positive surface charge may potentially suppress the release of the nucleic acid from the carrier due to interaction with the delivered nucleic acid within the cells, and may potentially suppress the protein synthesis due to the interaction between mRNA and the delivered nucleic acid. These problems can be solved by regulating the surface charge within the aforementioned range. The surface charge can be measured, for example, by using a zeta potential measuring device, such as Zetasizer Nano. The surface charge of the lipid membrane structure can be regulated by the composition of the constitutional components of the lipid membrane structure containing the ionizable lipid of the present invention.

The lipid membrane surface pKa of the lipid membrane structure of the present invention (which may be hereinafter abbreviated as a “liposomal pKa”) is not particularly limited, and is preferably 4.5 to 9.5. The liposomal pKa is considered as an index that indicates the susceptibility of a lipid membrane structure taken up through endocytosis to protonation under a weakly acidic environment in an endosome. As described in Angewandte Chemie International Edition, 51:8529-8533, 2012 or Molecular Therapy, 24(4): 786-795, 2016, in order to escape from an endosome and to deliver a nucleic acid into the cytoplasm, it is important that the liposomal pKa is a value that is preferred for the endosomal escape, and the delivery of the nucleic acid into the cytoplasm can be efficiently performed by regulating the liposomal pKa within the aforementioned range. The liposomal pKa can be regulated by the composition of the constitutional components of the lipid membrane structure containing the ionizable lipid.

By bringing the nucleic acid transfer agent of the present invention containing a nucleic acid (preferably the lipid membrane structure of the present invention containing a nucleic acid) into contact with cells, the nucleic acid contained in the nucleic acid transfer agent can be transferred into the cells. Accordingly, the present invention also provides a method for transferring a nucleic acid contained in the nucleic acid transfer agent into cells, including bringing the cells into contact with the nucleic acid transfer agent of the present invention containing a nucleic acid.

The type of the cells is not particularly limited, and prokaryotic and eukaryotic cells can be used, and eukaryotic cells are preferred. The type of the eukaryote is also not particularly limited, and examples thereof include vertebrates, such as mammals including humans (e.g., humans, monkeys, mice, rats, hamsters, and cows), birds (e.g., chickens and ostriches), amphibians (e.g., frogs), and fish (e.g., zebrafish and medaka), invertebrates, such as insects (e.g., silkworms, moths, and fruit flies), plants, and microorganisms (e.g., yeasts). The cells targeted by the present invention are more preferably animal or plant cells, and further preferably mammalian cells. The cells may be a cultured cell line including cancer cells, cells isolated from an individual or a tissue, or cells of a tissue or a tissue fragment. The cells may also be adherent cells or non-adherent cells.

The step of bringing cells into contact with the lipid membrane structure of the present invention containing a nucleic acid in vitro is specifically described below.

The cells are suspended in an appropriate culture medium several days before the contact with the lipid membrane structure, and cultured under an appropriate condition. At the time of contact with the lipid membrane structure, the cells may be or may not be in a growth phase.

The culture medium at the time of contact may be a serum-containing medium or a serum-free medium, in which the serum concentration in the medium is preferably 30% by weight or less, and more preferably 20% by weight or less. An excessive amount of proteins, such as serum, contained in the medium may possibly inhibit the contact between the lipid membrane structure and the cells.

The cell density at the time of contact is not particularly limited and can be appropriately set in consideration of the type of the cells and the like, and is generally in a range of 1×10⁴ to 1×10⁷ cells/mL.

For example, a suspension liquid of the lipid membrane structure of the present invention containing a nucleic acid (i.e., the nucleic acid transfer agent of the present invention containing a nucleic acid) is added to the cells. The amount of the suspension liquid added is not particularly limited, and can be appropriately determined in consideration of the number of the cells and the like. The concentration of the lipid membrane structure at the time of contact with the cells is not particularly limited, as long as the transfer of the intended nucleic acid into the cells can be achieved, and in terms of the lipid concentration, is generally 1 to 100 nmol/mL, and preferably 10 to 50 nmol/mL, and in terms of the nucleic acid concentration, is generally 0.01 to 100 μg/mL, and preferably 0.1 to 10 μg/mL.

After adding the suspension liquid to the cells, the cells are cultured. The temperature, the humidity, the CO₂ concentration, and the like during culture are appropriately set in consideration of the type of the cells. In the case where the cells are mammal-derived cells, in general, the temperature is approximately 37° C., the humidity is approximately 95%, and the CO₂ concentration is approximately 5% by volume. The culture time can also be appropriately set in consideration of the conditions, such as the type of the cells used, and is generally in a range of 0.1 to 76 hours, preferably in a range of 0.2 to 24 hours, and more preferably in a range of 0.5 to 12 hours. In the case where the culture time is too short, the nucleic acid is not sufficiently transferred into the cells, and in the case where the culture time is too long, the cells may possibly be weakened.

The nucleic acid is transferred into the cells by the culture described above, and it is preferred that the medium is replaced with a fresh medium, or a fresh medium is added to the medium, and the culture is further continued. In the case where the cells are mammal-derived cells, the fresh medium preferably contains serum or a trophic factor.

As described above, the use of the nucleic acid transfer agent of the present invention containing a nucleic acid enables the transfer of a nucleic acid into cells not only in vitro but also in vivo. Specifically, by administering the nucleic acid transfer agent of the present invention containing a nucleic acid to a living body, the nucleic acid contained in the nucleic acid transfer agent can be transferred into the target cell in the living body. Accordingly, the present invention also provides a method for transferring a nucleic acid contained in the nucleic acid transfer agent into target cells in a living body, including administering the nucleic acid transfer agent of the present invention containing the nucleic acid to the living body.

The living body to which the nucleic acid transfer agent of the present invention containing a nucleic acid can be administered is not particularly limited, and examples thereof include vertebrates such as mammals (e.g., humans, monkeys, mice, rats, hamsters, and cows), birds (e.g., chickens and ostriches), amphibians (e.g., frogs), and fish (e.g., zebrafish and medaka), invertebrates such as insects (e.g., silkworms, moths, and fruit flies), and plants. The living body to which the nucleic acid transfer agent of the present invention containing a nucleic acid is administered is preferably humans or another mammal.

The type of the target cells is not particularly limited, and the use of the nucleic acid transfer agent of the present invention containing a nucleic acid enables the transfer of the nucleic acid into cells in various tissues (e.g., liver, kidney, pancreas, lung, spleen, heart, blood, muscle, bone, brain, stomach, small intestine, large intestine, skin, adipose tissue, lymph node, and tumor).

The method of administering the nucleic acid transfer agent of the present invention containing a nucleic acid to a living body is not particularly limited, and administration method known per se (such as oral administration and parenteral administration (e.g., intravenous administration, intramuscular administration, topical administration, transdermal administration, subcutaneous administration, intraperitoneal administration, and spray)) can be appropriately selected. The dose of the nucleic acid transfer agent of the present invention containing a nucleic acid is not particularly limited, and can be appropriately selected in consideration of the type of the living body to be administered, the administration method, the type or the site of the target cells, and the like.

In the case where the ionizable lipid or the lipid membrane structure of the present invention is used as a nucleic acid transfer agent, the ionizable lipid or the lipid membrane structure can be formulated according to the ordinary method.

In the case where the nucleic acid transfer agent of the present invention, which is the lipid membrane structure of the present invention, is provided as a research reagent, the lipid membrane structure of the present invention can be provided as it is, or can be provided as a sterile solution or suspension liquid of the lipid membrane structure of the present invention, for example, with water or another physiologically acceptable liquid (such as an aqueous solvent (e.g., a malate buffer solution), an organic solvent (e.g., ethanol, methanol, DMSO, and tert-butanol), or a mixed solution of an aqueous solvent and an organic solvent). The nucleic acid transfer agent of the present invention may appropriately contain a physiologically acceptable additive known per se (such as an excipient, a vehicle, a preservative, a stabilizer, and a binder).

In the case where the nucleic acid transfer agent of the present invention, which is the lipid membrane structure of the present invention, is provided as a medicine, the lipid membrane structure of the present invention can be used as it is, or can be used with a known pharmaceutically acceptable additive (such as a carrier, a flavoring agent, an excipient, a vehicle, a preservative, a stabilizer, and a binder), and the nucleic acid transfer agent of the present invention can be produced as an oral preparation (such as a tablet and a capsule) or a parenteral preparation (such as an injection and a spray) by mixing in a unit dosage form required for generally accepted formulation practice, and preferably as a parenteral preparation (more preferably, an injection).

The nucleic acid transfer agent of the present invention can also be provided in the form of a kit. The kit may contain, in addition to the ionizable lipid of the present invention or the lipid membrane structure of the present invention described above, a reagent used at the time of the nucleic acid transfer. In one embodiment, the nucleic acid transfer agent of the present invention (or the kit) further contains a polycation (such as protamine). With the use of the nucleic acid transfer agent of the present invention (or the kit) of the embodiment, an electrostatic complex of a nucleic acid and a polycation (such as protamine) can be easily encapsulated in the lipid membrane structure of the present invention, and the nucleic acid can be subjected to the transfer into cells.

The present invention also provides a pharmaceutical composition containing the ionizable lipid of the present invention described above. The pharmaceutical composition of the present invention may further contain a nucleic acid. The pharmaceutical composition of the present invention may also contain a known pharmaceutically acceptable additive (such as a carrier, a flavoring agent, an excipient, a vehicle, a preservative, a stabilizer, and a binder).

The pharmaceutical composition of the present invention may be a powder composition obtained by removing the solvent through lyophilization or the like, or may be a liquid composition. The powder composition may also be produced by removing the solvent from the liquid composition through filtration, centrifugation, or the like, or may be produced through lyophilization of the liquid composition.

The pharmaceutical composition of the present invention may be produced as an oral preparation (such as a tablet and a capsule) or a parenteral preparation (such as an injection and a spray), and preferably as a parenteral preparation (more preferably, an injection). The pharmaceutical composition of the present invention can be produced not only as an adult formulation but also as a pediatric formulation.

The present invention also provides a method for producing a cellular medicine containing cells that express a gene in a nucleic acid, including bringing cells into contact with the nucleic acid transfer agent of the present invention containing a nucleic acid to transfer the nucleic acid contained in the nucleic acid transfer agent into the cells.

The cells that express a gene in the nucleic acid mean cells that express the intended gene by transferring the nucleic acid into the cells.

The nucleic acid can be transferred into the cell by bringing the cells into contact with the nucleic acid transfer agent of the present invention containing the nucleic acid in vitro.

Examples of the cells used in the production of the cellular medicine include T cells, B cells, NK cells, dendritic cells, macrophages, and monocytes. The T cells used in the production of the cellular medicine may be T cells differentiated from lymphocyte precursor cells including pluripotent cells into T cells. Examples of lymphocyte precursor cells including pluripotent cells include embryonic stem cells (ES cells) and induced pluripotent stem cells (iPS cells). Undifferentiated cells, such as pluripotent cells, can be differentiated into T cells by a known method.

Examples of the nucleic acid used in the production of the cellular medicine include a nucleic acid encoding a chimeric antigen receptor (CAR) and a nucleic acid encoding a T cell receptor (TCR).

The nucleic acid encoding a CAR used in the production of the cellular medicine includes an antigen-binding domain of an antibody that can specifically recognize a surface antigen that the target immune cells recognize, an extracellular hinge domain, a transmembrane domain, and an intracellular T cell signaling domain.

The nucleic acid encoding a TCR used in the production of the cellular medicine is a nucleic acid encoding a TCR α-chain and a TCR β-chain that can specifically recognize a surface antigen that the target T cells recognize.

The nucleic acid encoding a CAR or a TCR is not particularly limited, and examples thereof include DNA, RNA, a chimeric nucleic acid of RNA, and a DNA/RNA hybrid.

The cellular medicine contains the cells that express the specific gene, and may further contain a pharmaceutically acceptable additive (such as a carrier, an excipient, a vehicle, a preservative, and a stabilizer). The cellular medicine is preferably a parenteral preparation, and more preferably an injection.

The cellular medicine can be used for treatment or prevention of a disease, such as cancers. The cancers to which the cellular medicine is applicable are not particularly limited, and examples thereof include lung cancer, breast cancer, gastric cancer, colorectal cancer, uterine cancer, ovarian cancer, osteosarcoma, chondrosarcoma, rhabdomyosarcoma, leiomyosarcoma, fibrosarcoma, liposarcoma, angiosarcoma, leukemia, malignant lymphoma, and myeloma.

A subject to which the cellular medicine can be administered is not particularly limited, and examples thereof include mammals (such as humans, monkeys, mice, rats, hamsters, and cows). The subject to which the cellular medicine is administered is preferably humans or another mammal.

The method of administering the cellular medicine is not particularly limited, as long as being a method capable of allowing the cells to express the intended gene, and in consideration of the type of the cells, the target disease, and the like, for example, parenteral administration (such as intravenous administration, intramuscular administration, topical administration, transdermal administration, subcutaneous administration, intraperitoneal administration, and spray) can be appropriately selected. The dose of the cellular medicine is not particularly limited, as long as being within a range in which the cells can express the intended gene, and can be appropriately selected in consideration of the type of the subject to be administered, the administration method, the type of the cells, the target disease, and the like.

### EXAMPLES

The present invention is described in more detail with reference to Examples and Test Examples below, but the present invention is not limited thereto.

The meanings of the abbreviations used in the description of Examples are as follows.
Chol: cholesterol
DMAP: 4-dimethylaminopyridine
DMG-PEG2k: 1,2-dimyristoyl-rac-glycero-3-methoxypolyethylene glycol (number average molecular weight of PEG chain: 2,000)
DMSO: dimethylsulfoxide
DOPC: 1,2-dioleoyl-sn-glycero-3-phosphocholine
EDC: 1-ethyl-3-(3-dimethylaminopropyl) carbodiimide hydrochloride
LNP: lipid nanoparticles
Me: methyl
MES: 2-morpholinoethanesulfonic acid
mRNA: messenger RNA
PBS: phosphate buffer saline
TBS: tris buffer saline
siRNA: small interfering RNA
TNS: sodium 6-(p-toluidino)-2-naphthalenesulfonate

Preferred specific examples of the ionizable lipid of the present invention include the compounds described in Examples 1 to 142 in Tables 7-1 to 7-23 hereinafter, but the present invention is not construed as being limited thereto. The compounds described in Examples 1 to 142 each are referred to as “BET” and an associated number therefor. In Comparative Example 4, a reagent purchased from Fujifilm Wako Pure Chemical Corporation is used.

TABLE 7-1

| Example | Name | Strucure |
|---|---|---|
| 1 | BET1 | 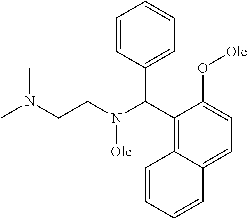 |
| 2 | BET2 | 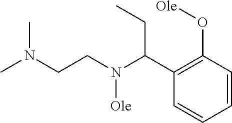 |
| 3 | BET3 | 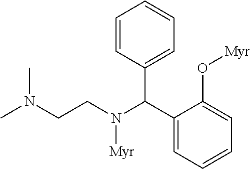 |
| 4 | BET4 | 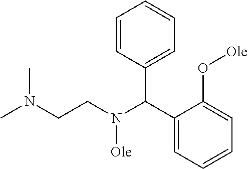 |
| 5 | BET5 | 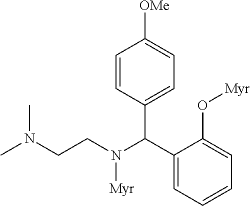 |
| 6 | BET6 | 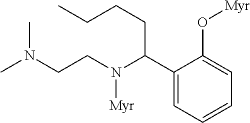 |
| 7 | BET7 | 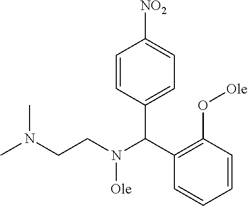 |
| 8 | BET8 | 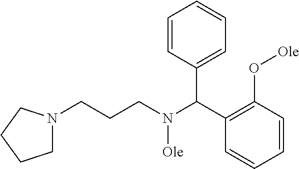 |

TABLE 7-2

| | | |
|---|---|---|
| 9 | BET9 | 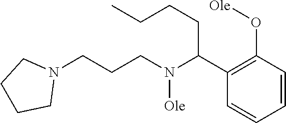 |
| 10 | BET10 | 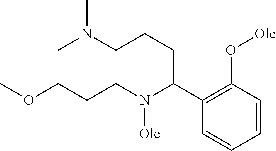 |
| 11 | BET11 | 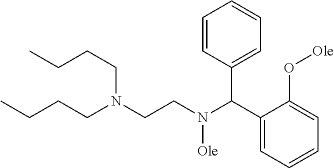 |
| 12 | BET12 | 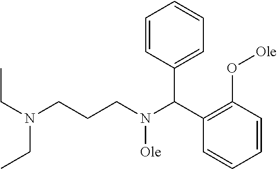 |
| 13 | BET13 | 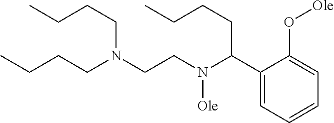 |
| 14 | BET14 | 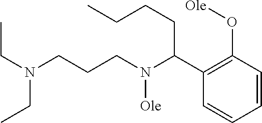 |
| 15 | BET15 | 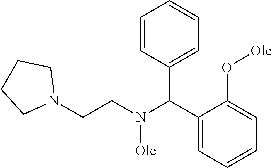 |
| 16 | BET16 | 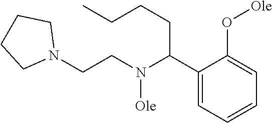 |
| 17 | BET17 | 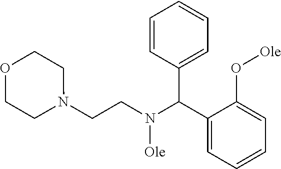 |

TABLE 7-3

| | | |
|---|---|---|
| 18 | BET18 | 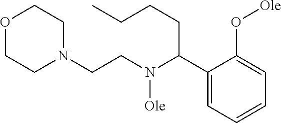 |
| 19 | BET19 | 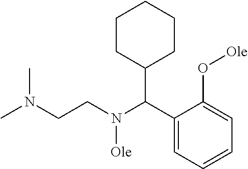 |
| 20 | BET20 | 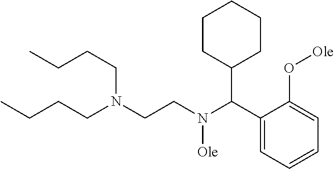 |
| 21 | BET21 | 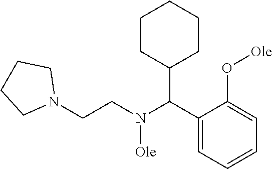 |
| 22 | BET22 | 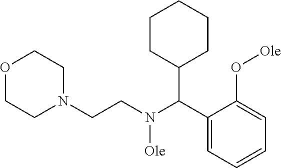 |
| 23 | BET23 | 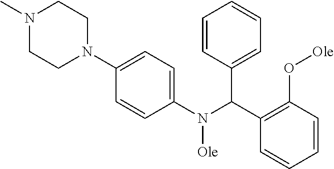 |
| 24 | BET24 | 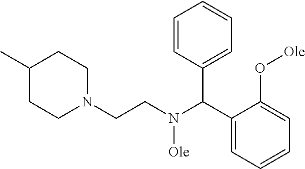 |
| 25 | BET25 | 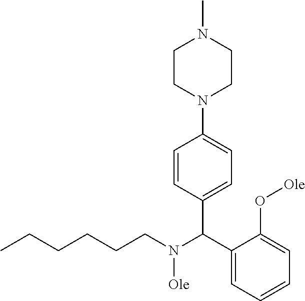 |

TABLE 7-4

| | | |
|---|---|---|
| 26 | BET26 | 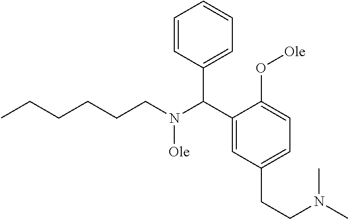 |
| 27 | BET27 | 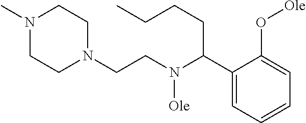 |
| 28 | BET28 | 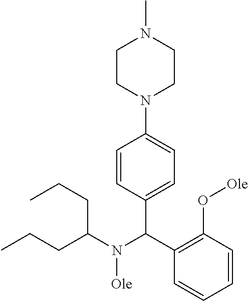 |
| 29 | BET29 | 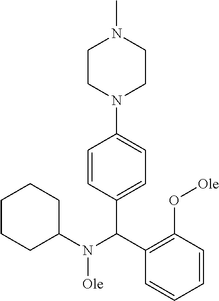 |
| 30 | BET30 | 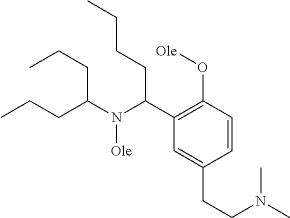 |
| 31 | BET31 | 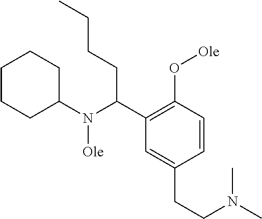 |

TABLE 7-5

| | | |
|---|---|---|
| 32 | BET82 | 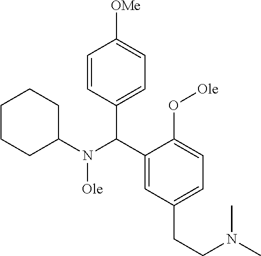 |
| 33 | BET33 | 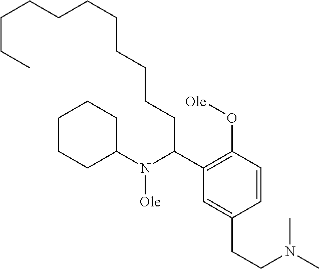 |
| 34 | BET34 | 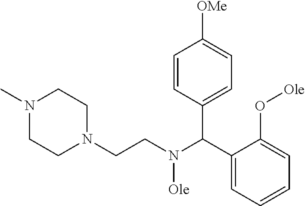 |
| 35 | BET35 | 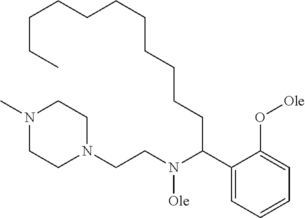 |
| 36 | BET36 | 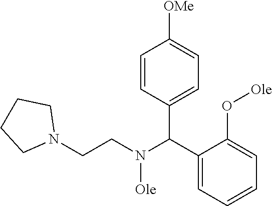 |
| 37 | BET37 | 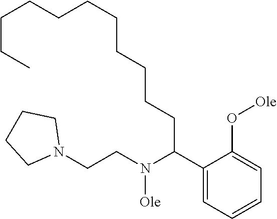 |

TABLE 7-6

| | | |
|---|---|---|
| 38 | BET38 | 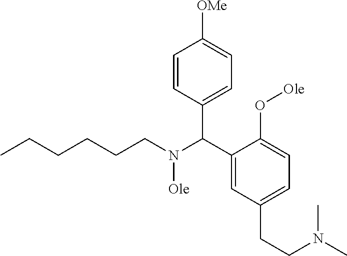 |
| 39 | BET39 | 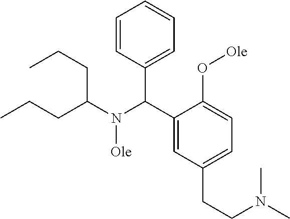 |
| 40 | BET40 | 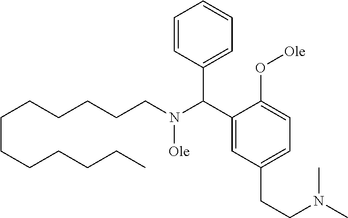 |
| 41 | BET41 | 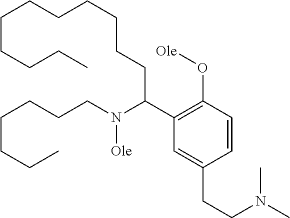 |
| 42 | BET42 | 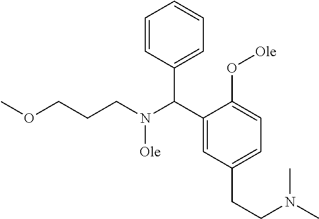 |
| 43 | BET43 | 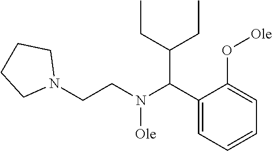 |

TABLE 7-7

| | | |
|---|---|---|
| 44 | BET44 | 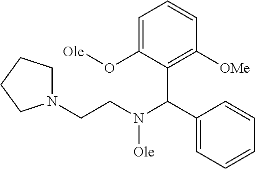 |
| 45 | BET46 | 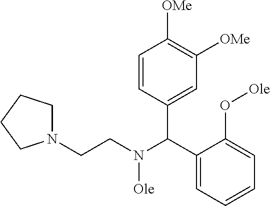 |
| 46 | BET46 | 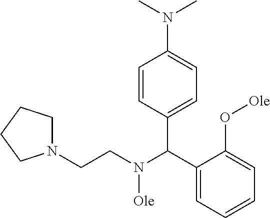 |
| 47 | BET47 | 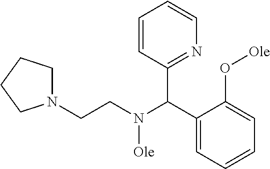 |
| 48 | BET48 | 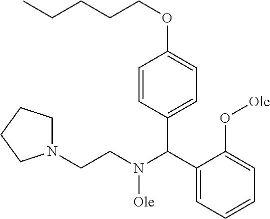 |
| 49 | BET49 | 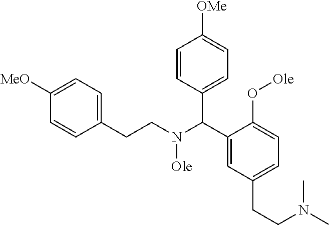 |

TABLE 7-8

| | | |
|---|---|---|
| 50 | BET50 | 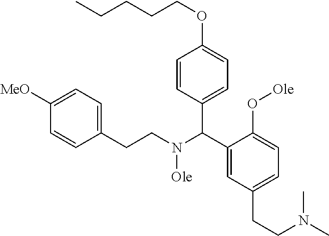 |
| 51 | BET51 | 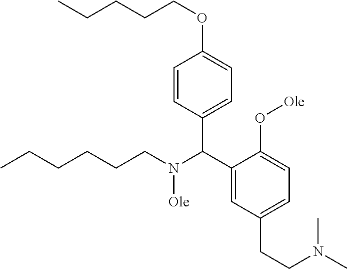 |
| 52 | BET52 | 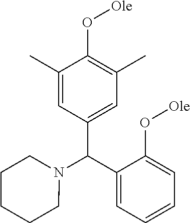 |
| 53 | BET53 | 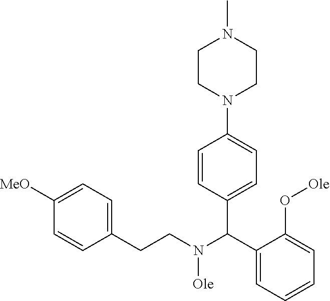 |
| 54 | BET54 | 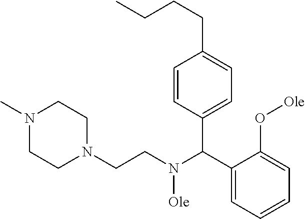 |

TABLE 7-9

| | | |
|---|---|---|
| 55 | BET55 | 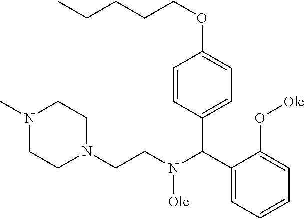 |
| 56 | BET56 | 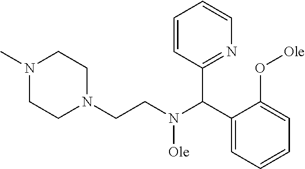 |
| 57 | BET57 | 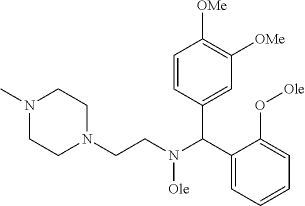 |
| 58 | BET58 | 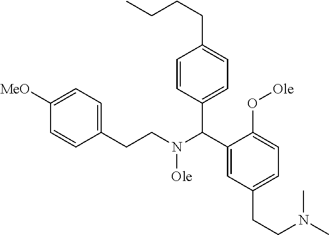 |
| 59 | BET59 | 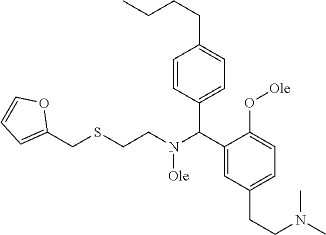 |
| 60 | BET60 | 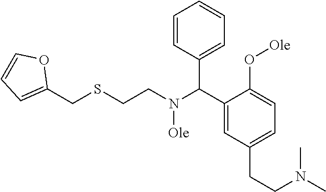 |

TABLE 7-10

| | | |
|---|---|---|
| 61 | BET61 | 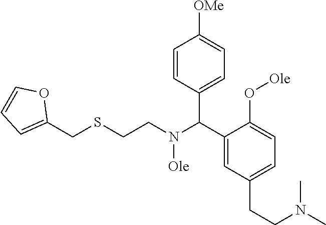 |
| 62 | BET62 | 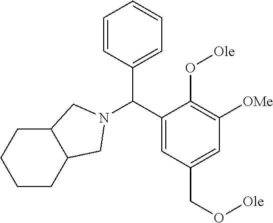 |
| 63 | BET63 | 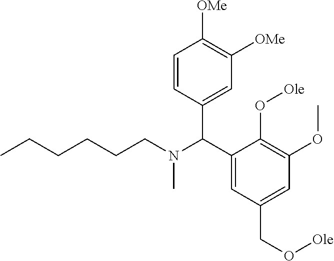 |
| 64 | BET64 | 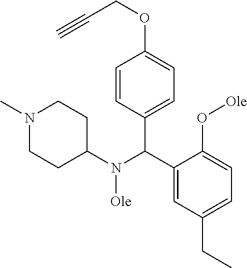 |
| 65 | BET65 | 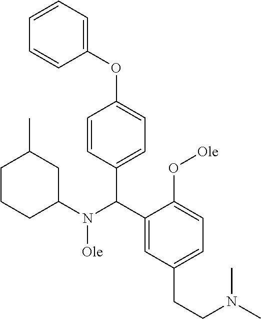 |

TABLE 7-11

| | | |
|---|---|---|
| 66 | BET66 | 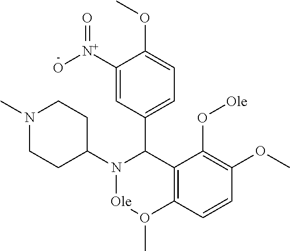 |
| 67 | BET67 | 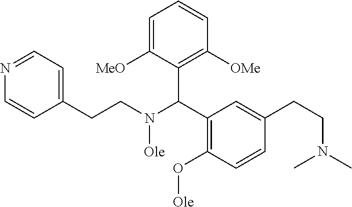 |
| 68 | BET68 | 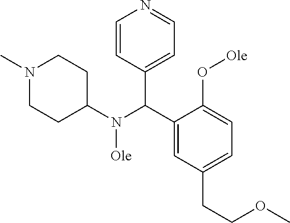 |
| 69 | BET69 | 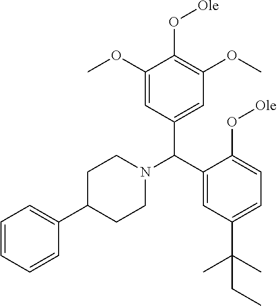 |
| 70 | BET70 | 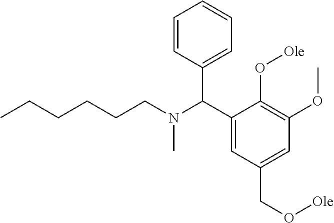 |
| 71 | BET71 | 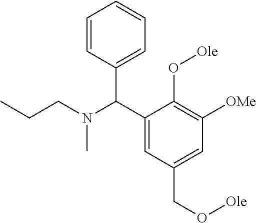 |

TABLE 7-12

| | | |
|---|---|---|
| 72 | BET072 | 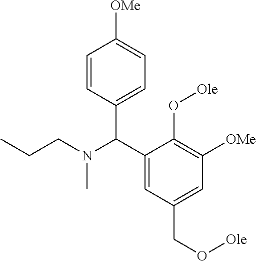 |
| 73 | BET073 | 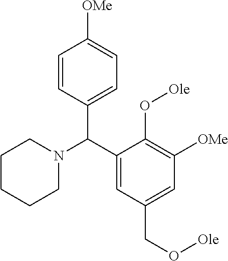 |
| 74 | BET074 | 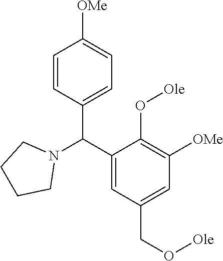 |
| 75 | BET075 | 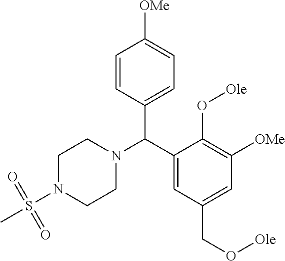 |
| 76 | BET076 | 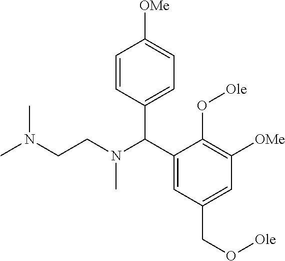 |
| 77 | BET077 | 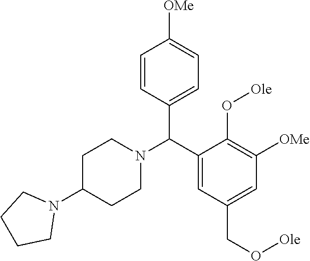 |

TABLE 7-13

| | | |
|---|---|---|
| 78 | BET078 | 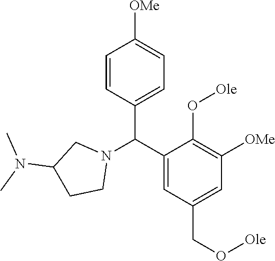 |
| 79 | BET079 | 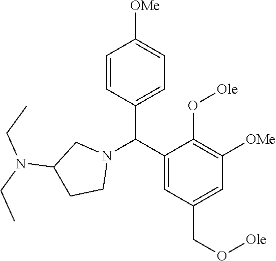 |
| 80 | BET080 | 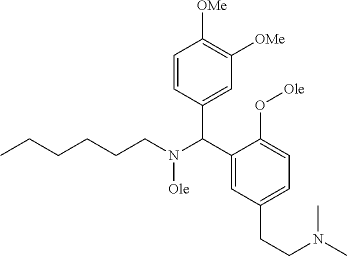 |
| 81 | BET081 | 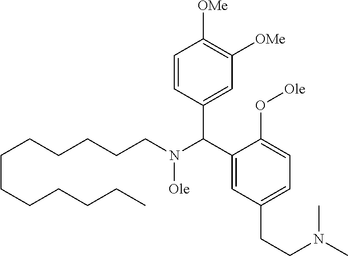 |
| 82 | BET082 | 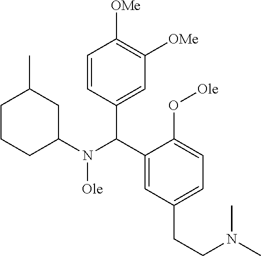 |
| 83 | BET083 | 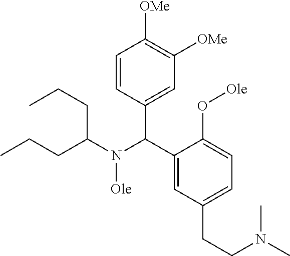 |

TABLE 7-14

| | | |
|---|---|---|
| 84 | BET084 | 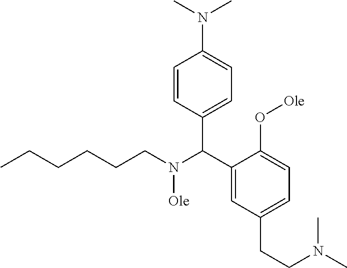 |
| 85 | BET085 | 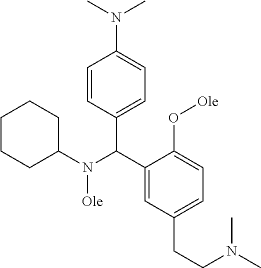 |
| 86 | BET086 | 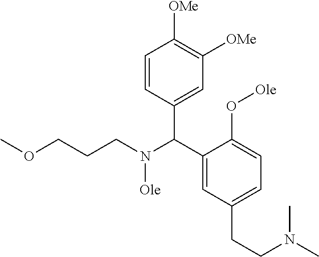 |
| 87 | BET087 | 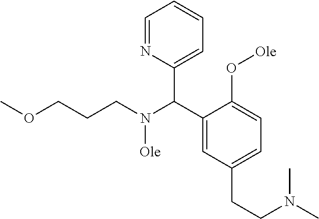 |
| 88 | BET088 | 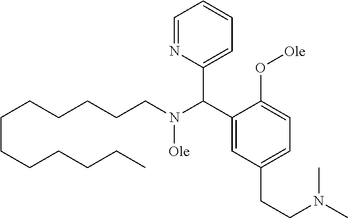 |
| 89 | BET089 | 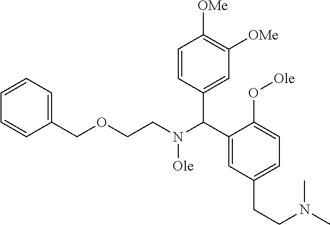 |

TABLE 7-15

| | | |
|---|---|---|
| 90 | BET090 | 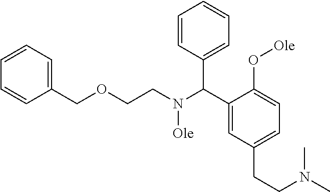 |
| 91 | BET091 | 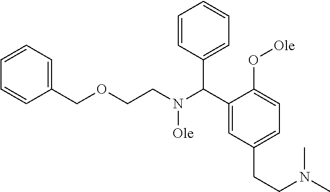 |
| 92 | BET092 | 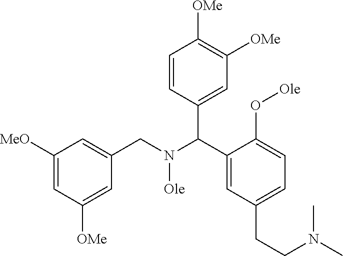 |
| 93 | BET093 | 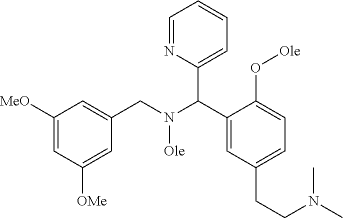 |
| 94 | BET094 | 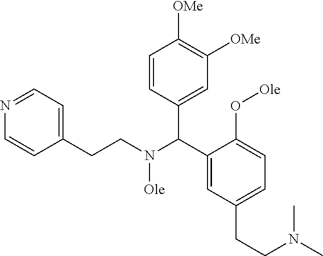 |
| 95 | BET095 | 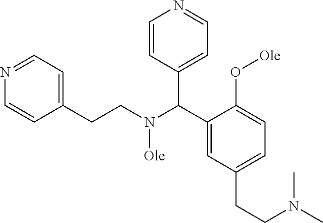 |

TABLE 7-16

| | | |
|---|---|---|
| 96 | BET096 | 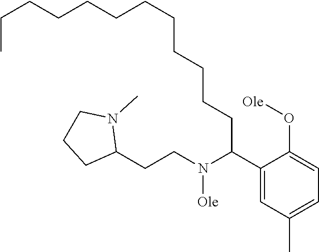 |
| 97 | BET097 | 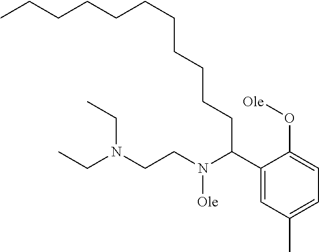 |
| 98 | BET098 | 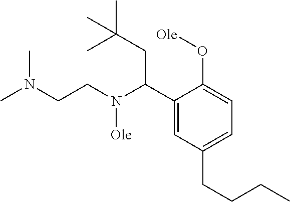 |
| 99 | BET099 | 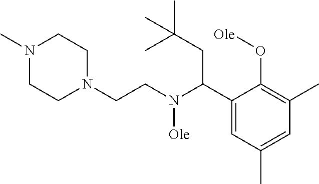 |
| 100 | BET100 | 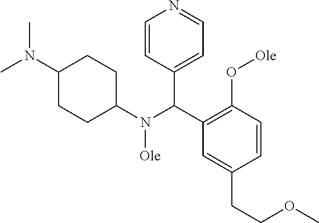 |
| 101 | BET101 | 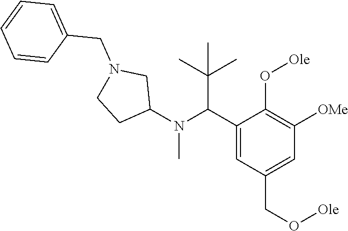 |

TABLE 7-17

| | | |
|---|---|---|
| 102 | BET102 | 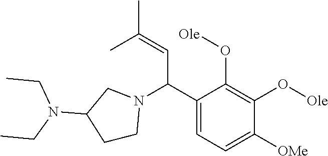 |
| 103 | BET103 | 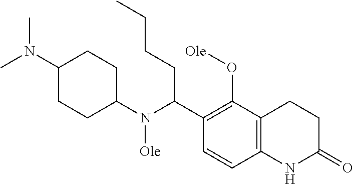 |
| 104 | BET104 | 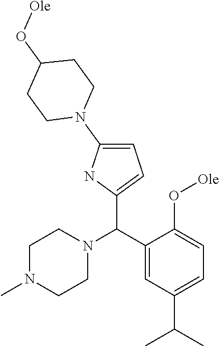 |
| 105 | BET105 | 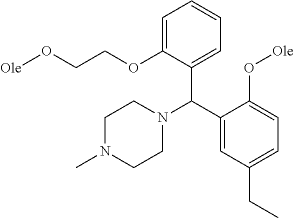 |
| 106 | BET106 | 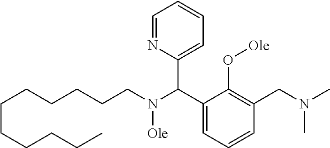 |
| 107 | BET107 | 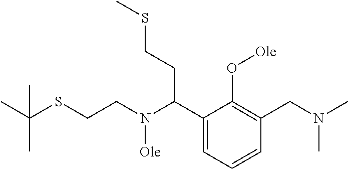 |

TABLE 7-18

| | | |
|---|---|---|
| 108 | BET108 | 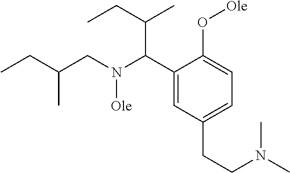 |
| 109 | BET109 | 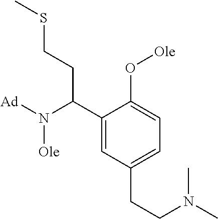 |
| 110 | BET110 | 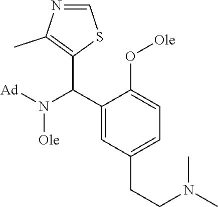 |
| 111 | BET111 | 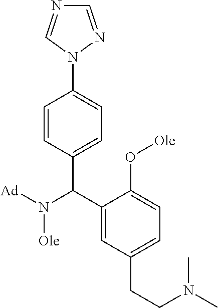 |
| 112 | BET112 | 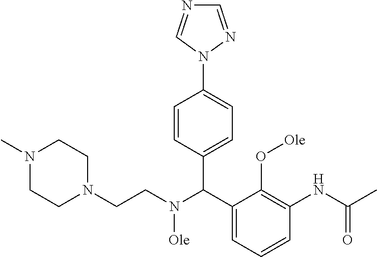 |
| 113 | BET113 | 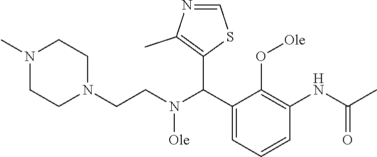 |

TABLE 7-19

| | | |
|---|---|---|
| 114 | BET114 | 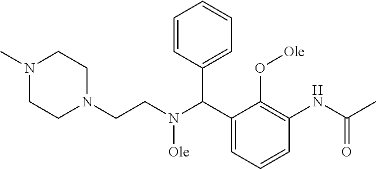 |
| 115 | BET115 | 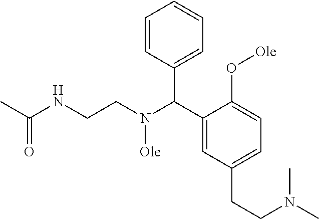 |
| 116 | BET116 | 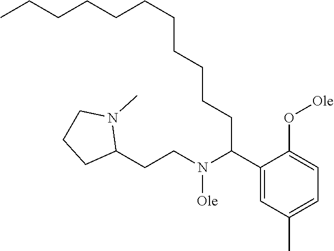 |
| 117 | BET117 | 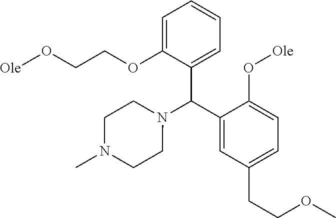 |
| 118 | BET118 | 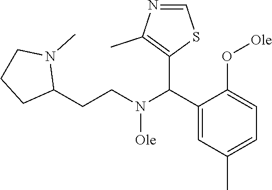 |
| 119 | BET119 | 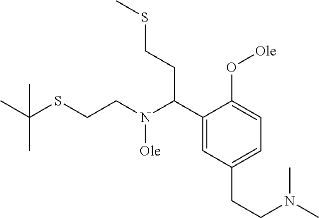 |

TABLE 7-20

| | | |
|---|---|---|
| 120 | BET120 | 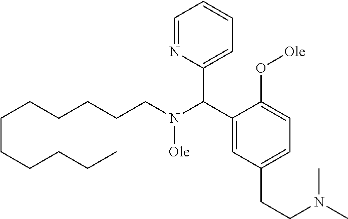 |
| 121 | BET121 | 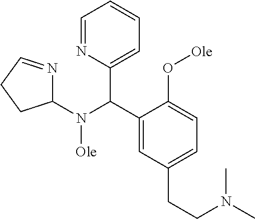 |
| 122 | BET122 | 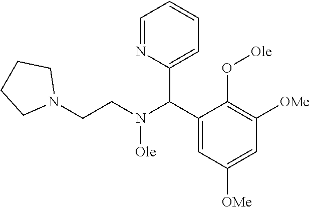 |
| 123 | BET123 | 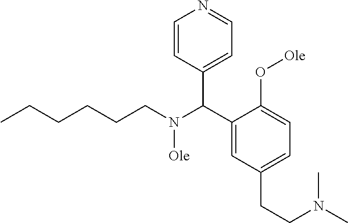 |
| 124 | BET124 | 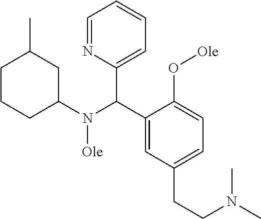 |
| 125 | BET125 | 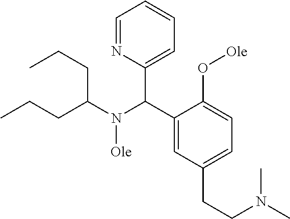 |

TABLE 7-21

| | | |
|---|---|---|
| 126 | BET126 | 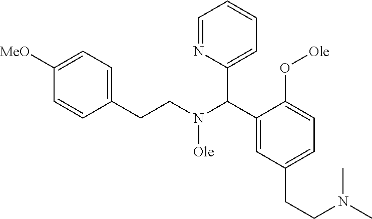 |
| 127 | BET127 | 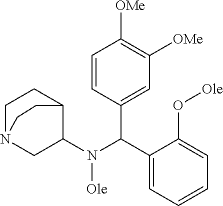 |
| 128 | BET128 | 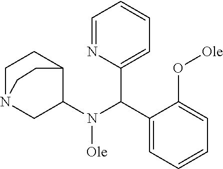 |
| 129 | BET129 | 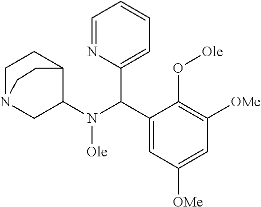 |
| 130 | BET130 | 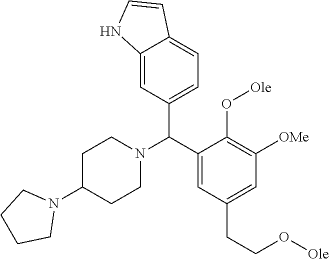 |
| 131 | BET131 | 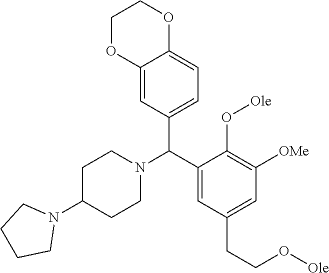 |

TABLE 7-22

| | | |
|---|---|---|
| 132 | BET132 | 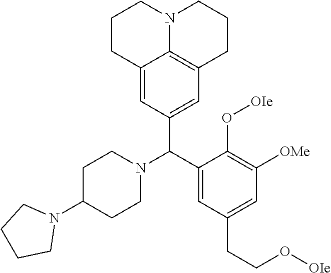 |
| 133 | BET133 | 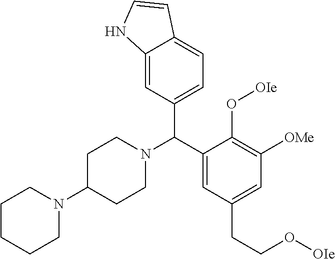 |
| 134 | BET134 | 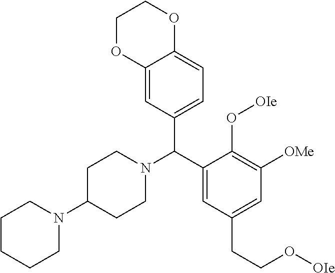 |
| 135 | BET135 | 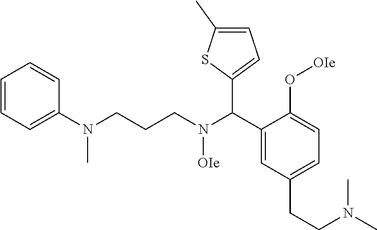 |
| 136 | BET136 | 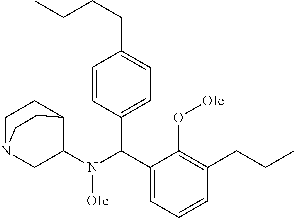 |
| 137 | BET137 | 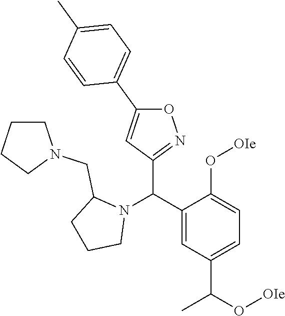 |

TABLE 7-23

| | | |
|---|---|---|
| 138 | BET138 | 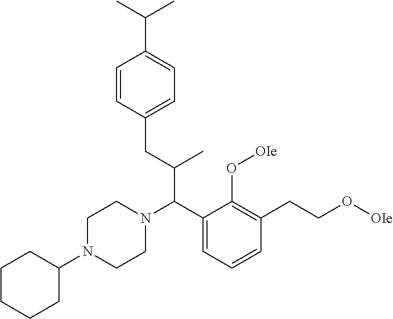 |
| 139 | BET139 | 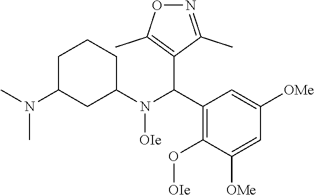 |
| 140 | BET140 | 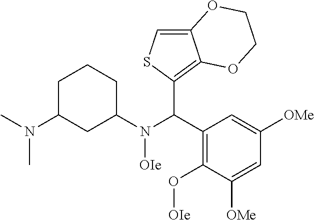 |
| 141 | BET141 | 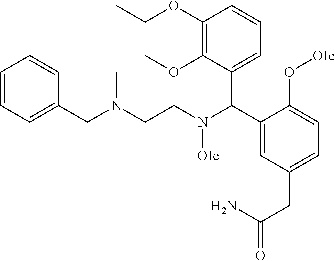 |
| 142 | BET142 | 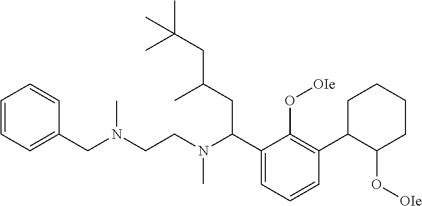 |

In Tables 7-1 to 7-23, Ole represents *—C(═O)—(CH₂)₇CH═CH(CH₂)₇CH₃, Myr represents *—C(═O)—C₁₃H₂₇, Ad represents adamantane, and * represents a bonding position.

TABLE 8

| Name | Structure |
|---|---|
| Comparative Example 1 | 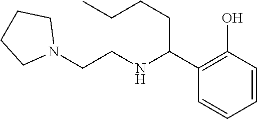 [SMILES: CCCCC(NCCN1CCCC1)c1ccccc1O] |
| Comparative Example 2 | 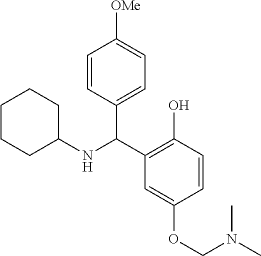 [SMILES: COc1ccc(C(NC2CCCCC2)c2cc(OCN(C)C)ccc2O)cc1] |
| Comparative Example 3 | 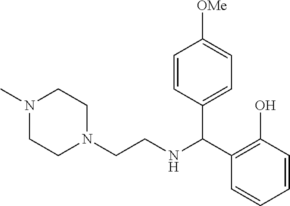 [SMILES: COc1ccc(C(NCCN2CCN(C)CC2)c2ccccc2O)cc1] |
| Comparative Example 4 | 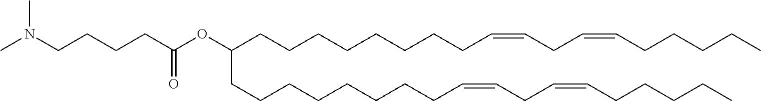 [SMILES: CCCCC/C=C\C/C=C\CCCCCCCCC(CCCCCCCC/C=C\C/C=C\CCCCC)OC(=O)CCCCN(C)C] |

### Example 1

### Synthesis of BET 1

0.71 g (8.00 mmol) of N,N-dimethylethylenediamine, 0.93 g (8.80 mmol) of benzaldehyde, and 2.31 g (16.0 mmol) of 2-naphthol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.83 g of the target product.

<¹H-NMR (600 MHz, CDCl₃) of Compound BET 1>

δ: 0.83-1.01 (m, 6H), 1.13-1.49 (m, 44H), 1.64-1.89 (m, 8H), 1.95-2.07 (m, 10H), 2.38-2.45 (m, 1H), 2.57-2.65 (m, 1H), 3.59-3.67 (m, 1H), 3.69-3.76 (m, 1H), 5.31-5.40 (m, 4H), 7.04 (d, 2H), 7.15 (d, 1H), 7.19 (t, 1H), 7.33 (t, 2H), 7.49-7.59 (m, 2H), 7.83-7.93 (m, 3H), 8.14 (d, 1H)

### Example 2

### Synthesis of BET 2

0.71 g (8.00 mmol) of N,N-dimethylethylenediamine, 0.51 g (8.80 mmol) of propionaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.84 g of the target product.

<¹H-NMR (600 MHz, CDCl₃) of Compound BET 2>

δ: 0.89 (t, 6H), 0.96 (t, 3H), 1.26-1.40 (m, 40H), 1.53 (m, 2H), 1.66 (m, 2H), 1.88 (m, 2H), 2.02-2.05 (m, 10H), 2.21 (s, 6H), 2.57 (t, 2H), 2.62 (t, 2H), 3.30 (t, 2H), 4.78 (t, 1H), 5.34 (m, 4H), 7.10 (dd, 1H), 7.22-7.30 (m, 3H)

### Example 3

### Synthesis of BET 3

0.71 g (8.00 mmol) of N,N-dimethylethylenediamine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.19 g (9.60 mmol) of myristic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.77 g of the target product.

<¹H-NMR (600 MHz, CDCl₃) of Compound BET 3>

δ: 0.81-0.94 (m, 6H), 1.12-1.42 (m, 41H), 1.44-1.84 (m, 5H), 1.90-1.99 (m, 6H), 2.09-2.16 (m, 0.5H), 2.19-2.33 (m, 1.5H), 2.33-2.51 (m, 2H), 3.32-3.40 (m, 1H), 3.47 (dt, 0.5H), 3.60 (dt, 0.5H), 6.31 (s, 0.5H), 7.06-7.20 (m, 4.5H), 7.23-7.39 (m, 5H)

### Example 4

### Synthesis of BET 4

0.71 g (8.00 mmol) of N,N-dimethylethylenediamine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.9 g of the target product.

<¹H-NMR (600 MHz, CDCl₃) of Compound BET 4>

δ: 0.83-0.92 (m, 6H), 1.20-1.40 (m, 40H), 1.42-1.58 (m, 3H), 1.68-1.81 (m, 2H), 1.90-2.06 (m, 15H), 2.07-2.15 (m, 0.5H), 2.18-2.51 (m, 3.5H), 3.30-3.40 (m, 1H), 3.42-3.50 (m, 0.5H), 3.62 (dt, 0.5H), 5.28-5.39 (m, 4H), 6.30 (s, 0.5H), 7.05-7.20 (m, 4.5H), 7.22-7.40 (m, 5H)

### Example 5

### Synthesis of BET 5

0.71 g (8.00 mmol) of N,N-dimethylethylenediamine, 1.2 g (8.80 mmol) of 4-methoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.19 g (9.60 mmol) of myristic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.81 g of the target product.

<¹H-NMR (600 MHz, CDCl₃) of Compound BET 5>

δ: 0.89 (t, 6H), 1.26-1.33 (m, 40H), 1.53 (m, 2H), 1.66 (m, 2H), 2.05 (t, 2H), 2.21 (s, 6H), 2.57 (t, 2H), 2.62 (t, 2H), 3.30 (t, 2H), 3.80 (s, 3H), 6.16 (s, 1H), 6.89 (d, 2H), 7.10 (dd, 1H), 7.21 (d, 2H), 7.30-7.35 (m, 2H), 7.44 (dd, 1H)

### Example 6

### Synthesis of BET 6

0.71 g (8.00 mmol) of N,N-dimethylethylenediamine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.19 g (9.60 mmol) of myristic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.75 g of the target product.

<¹H-NMR (600 MHz, CDCl₃) of Compound BET 6>

δ: 0.81-0.94 (m, 9H), 1.12-1.42 (m, 42H), 1.49 (dt, 1H), 1.57-1.77 (m, 5H), 1.79-1.89 (m, 2H), 2.00-2.16 (m, 7H), 2.18-2.33 (m, 2H), 2.51-2.65 (m, 2H), 3.00 (dt, 1H), 3.18 (dt, 1H), 6.03 (t, 1H), 7.02 (d, 1H), 7.22-7.29 (m, 1H), 7.31-7.36 (m, 1H), 7.41 (t, 1H), 7.48 (d, 1H)

### Example 7

### Synthesis of BET 7

0.71 g (8.00 mmol) of N,N-dimethylethylenediamine, 1.33 g (8.80 mmol) of 4-nitrobenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.95 g of the target product.

<¹H-NMR (600 MHz, CDCl₃) of Compound BET 7>

δ: 0.83-0.92 (m, 6H), 1.20-1.40 (m, 40H), 1.42-1.58 (m, 3H), 1.68-1.81 (m, 2H), 1.90-2.06 (m, 15H), 2.07-2.15 (m, 0.5H), 2.18-2.51 (m, 3.5H), 3.30-3.40 (m, 1H), 3.42-3.50 (m, 0.5H), 3.62 (dt, 0.5H), 5.28-5.39 (m, 4H), 6.30 (s, 0.5H), 7.05-7.20 (m, 3.5H), 7.22-7.50 (m, 5H)

### Example 8

### Synthesis of BET 8

1.03 g (8.00 mmol) of 1-(3-aminopropyl) pyrrolidine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.94 g of the target product.

<¹H-NMR (600 MHz, CDCl₃) of Compound BET 8>

δ: 0.89 (t, 6H), 1.26-1.40 (m, 40H), 1.53 (m, 2H), 1.65-1.68 (m, 8H), 2.02-2.05 (m, 10H), 2.36 (t, 2H), 2.51 (t, 4H), 2.57 (t, 2H), 3.18 (t, 2H), 5.34 (m, 4H), 6.16 (s, 1H), 7.10 (dd, 3H), 7.22 (dd, 1H), 7.29-7.35 (m, 4H), 7.44 (dd, 1H)

### Example 9

### Synthesis of BET 9

1.03 g (8.00 mmol) of 1-(3-aminopropyl) pyrrolidine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.92 g of the target product.

<¹H-NMR (600 MHz, CDCl₃) of Compound BET 9>

δ: 0.81-0.94 (m, 12H), 1.20-1.40 (m, 48H), 1.44-1.77 (m, 8H), 1.77-1.96 (m, 8H), 2.10-2.55 (m, 10H), 3.38-3.61 (m, 1.5H), 3.64-3.73 (0.5H), 6.03 (t, 1H), 7.02 (d, 1H), 7.22-7.29 (m, 1H), 7.31-7.36 (m, 1H), 7.41 (t, 1H), 7.48 (d, 1H)

### Example 10

### Synthesis of BET 10

0.71 g (8.00 mmol) of 3-methoxypropylamine, 1.42 g (8.80 mmol) of 4-(dimethylamino)butyraldehyde dimethyl acetal, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.91 g of the target product.

<¹H-NMR (600 MHz, CDCl₃) of Compound BET 10>

δ: 0.89 (t, 6H), 1.26-1.40 (m, 42H), 1.53 (m, 2H), 1.66-1.72 (m, 4H), 1.82 (m, 2H), 2.02-2.05 (m, 10H), 2.15 (s, 6H), 2.43 (t, 2H), 2.57 (t, 2H), 3.18 (t, 2H), 3.35 (t, 2H), 3.40 (s, 3H), 4.78 (t, 1H), 5.34 (m, 4H), 7.10 (dd, 1H), 7.22-7.30 (m, 3H)

### Example 11

### Synthesis of BET 11

1.38 g (8.00 mmol) of N,N-dibutylethylenediamine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 11>

δ: 0.89-0.91 (m, 12H), 1.26-1.41 (m, 48H), 1.53 (m, 2H), 1.66 (m, 2H), 2.02-2.05 (m, 10H), 2.38 (t, 4H), 2.57 (t, 2H), 2.62 (t, 2H), 3.30 (t, 2H), 5.34 (m, 4H), 6.16 (s, 1H), 7.10 (dd, 3H), 7.22 (dd, 1H), 7.29-7.35 (m, 4H), 7.44 (dd, 1H)

### Example 12

### Synthesis of BET 12

1.04 g (8.00 mmol) of N,N-diethyl-1,3-diaminopropane, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.94 g of the target product.

<¹H-NMR (600 MHz, CDCl₃) of Compound BET 12>

δ: 0.83-0.92 (m, 6H), 1.20-1.40 (m, 40H), 1.45-1.78 (m, 10H), 1.78-1.94 (m, 9H), 2.10-2.55 (m, 11H), 3.38-3.61 (m, 1.5H), 3.64-3.73 (0.5H), 5.28-5.40 (m, 4H), 6.32 (s, 0.5H), 7.03-7.20 (m, 4.5H), 7.22-7.40 (m, 5H)

### Example 13

### Synthesis of BET 13

1.38 g (8.00 mmol) of N,N-dibutylethylenediamine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.97 g of the target product.

<¹H-NMR (600 MHz, CDCl₃) of Compound BET 13>

δ: 0.81-0.94 (m, 15H), 1.14-1.46 (m, 52H), 1.55-1.77 (m, 5H), 1.78-1.88 (m, 2H), 1.96-2.07 (m, 8H), 2.14-2.39 (m, 7H), 2.51-2.66 (m, 2H), 2.94-3.03 (m, 1H), 3.04-3.11 (m, 1H), 5.30-5.38 (m, 4H), 5.99 (t, 1H), 7.02 (d, 1H), 7.24 (d, 1H), 7.32 (t, 1H), 7.44 (d, 1H)

### Example 14

### Synthesis of BET 14

1.04 g (8.00 mmol) of N,N-diethyl-1,3-diaminopropane, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.92 g of the target product.

<¹H-NMR (600 MHz, CDCl₃) of Compound BET 14>

δ: 0.89 (t, 9H), 1.15 (t, 6H), 1.25-1.40 (m, 44H), 1.53 (m, 2H), 1.66 (m, 4H), 1.82 (m, 2H), 2.02-2.05 (m, 10H), 2.36 (t, 2H), 2.57 (t, 2H), 3.01 (q, 4H), 3.18 (t, 2H), 4.78 (t, 1H), 5.34 (m, 4H), 7.10 (dd, 1H), 7.22-7.25 (m, 2H), 7.30 (m, 1H)

### Example 15

### Synthesis of BET 15

0.91 g (8.00 mmol) of 1-(2-aminoethyl) pyrrolidine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.92 g of the target product.

<¹H-NMR (600 MHz, CDCl₃) of Compound BET 15>

δ: 0.83-0.92 (m, 6H), 1.20-1.40 (m, 40H), 1.45-1.78 (m, 8H), 1.78-1.94 (m, 9H), 2.10-2.55 (m, 9H), 3.38-3.61 (m, 1.5H), 3.64-3.73 (0.5H), 5.28-5.40 (m, 4H), 6.32 (s, 0.5H), 7.03-7.20 (m, 4.5H), 7.22-7.40 (m, 5H)

### Example 16

### Synthesis of BET 16

0.91 g (8.00 mmol) of 1-(2-aminoethyl) pyrrolidine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.9 g of the target product.

<¹H-NMR (600 MHz, CDCl₃) of Compound BET 16>

δ: 0.84-0.94 (m, 9H), 1.18-1.45 (m, 44H), 1.55-1.78 (m, 8H), 1.80-1.88 (m, 2H), 1.94-2.08 (m, 8H), 2.19-2.26 (m, 1H), 2.29-2.65 (m, 9H), 3.07-3.15 (m, 1H), 3.18-3.30 (m, 1H), 5.30-5.39 (m, 4H), 6.03 (t, 1H), 7.01 (d, 1H), 7.24 (t, 1H), 7.32 (t, 1H), 7.45 (d, 1H)

### Example 17

### Synthesis of BET 17

1.04 g (8.00 mmol) of 4-(2-aminoethyl) morpholine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.94 g of the target product.

<¹H-NMR (600 MHz, CDCl₃) of Compound BET 17>

δ: 0.89 (t, 6H), 1.26-1.40 (m, 40H), 1.53 (m, 2H), 1.66 (m, 2H), 2.02-2.05 (m, 10H), 2.40 (t, 4H), 2.57 (t, 2H), 2.62 (t, 2H), 3.30 (t, 2H), 3.52 (t, 4H), 5.34 (m, 4H), 6.16 (s, 1H), 7.10 (dd, 3H), 7.22 (dd, 1H), 7.29-7.35 (m, 4H), 7.44 (m, 1H)

### Example 18

### Synthesis of BET 18

1.04 g (8.00 mmol) of 4-(2-aminoethyl) morpholine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.92 g of the target product.

<¹H-NMR (600 MHz, CDCl₃) of Compound BET 18>

δ: 0.83-0.94 (m, 9H), 1.12-1.45 (m, 42H), 1.48-1.78 (m, 8H), 1.79-1.88 (m, 2H), 1.95-2.07 (m, 8H), 2.13-2.36 (m, 6H), 2.44-2.65 (m, 2H), 3.03-3.19 (m, 2H), 3.55-3.64 (m, 4H), 5.30-5.40 (m, 4H), 6.03 (m, 1H), 7.01 (d, 1H), 7.21-7.41 (m, 2H), 7.46 (d, 1H)

### Example 19

### Synthesis of BET 19

0.71 g (8.00 mmol) of N,N-dimethylethylenediamine, 0.99 g (8.80 mmol) of cyclohexanecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.9 g of the target product.

<¹H-NMR (600 MHz, CDCl₃) of Compound BET 19>

δ: 0.83-0.95 (m, 6H), 1.11-1.44 (m, 51H), 1.51-1.83 (m, 7H), 1.97-2.13 (m, 14H), 2.13-2.22 (m, 1H), 2.29 (t, 1H), 2.62 (t, 1H), 3.02-3.11 (m, 1H), 3.25-3.32 (m, 1H), 5.28-5.38 (m, 4H), 5.80 (d, 1H), 7.00 (d, 1H), 7.24 (t, 1H), 7.32 (t, 1H), 7.45 (d, 1H)

### Example 20

### Synthesis of BET 20

1.38 g (8.00 mmol) of N,N-dibutylethylenediamine, 0.99 g (8.80 mmol) of cyclohexanecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.0 g of the target product.

<¹H-NMR (600 MHz, CDCl₃) of Compound BET 20>

δ: 0.89-0.91 (t, 12H), 1.26-1.60 (m, 62H), 2.02-2.05 (m, 10H), 2.38 (t, 4H), 2.44 (m, 1H), 2.57 (t, 2H), 2.62 (t, 2H), 3.30 (t, 2H), 4.68 (s, 1H), 5.34 (m, 4H), 7.10 (dd, 1H), 7.22-7.25 (m, 2H), 7.30 (ddd, 1H)

### Example 21

### Synthesis of BET 21

0.91 g (8.00 mmol) of 1-(2-aminoethyl) pyrrolidine, 0.99 g (8.80 mmol) of cyclohexanecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.93 g of the target product.

<¹H-NMR (600 MHz, CDCl₃) of Compound BET 21>

δ: 0.83-0.96 (m, 6H), 1.09-1.44 (m, 44H), 1.47-1.87 (m, 15H), 1.95-2.07 (m, 9H), 2.15-2.41 (m, 6H), 2.52-2.67 (m, 3H), 3.06-3.14 (m, 1H), 3.25-3.36 (m, 1H), 5.30-5.41 (m, 4H), 5.80 (d, 1H), 6.97-7.06 (m, 1H), 7.20-7.39 (m, 2H), 7.46 (d, 1H)

### Example 22

### Synthesis of BET 22

1.04 g (8.00 mmol) of 4-(2-aminoethyl) morpholine, 0.99 g (8.80 mmol) of cyclohexanecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.95 g of the target product.

<¹H-NMR (600 MHz, CDCl₃) of Compound BET 22>

δ: 0.83-1.00 (m, 7H), 1.10-1.45 (m, 45H), 1.50-1.84 (m, 10H), 1.95-2.14 (m, 10H), 2.15-2.35 (m, 5H), 2.50-2.65 (m, 2H), 3.03-3.11 (m, 1H), 3.21-3.31 (m, 1H), 3.56-3.66 (m, 4H), 5.30-5.41 (m, 4H), 5.80 (d, 1H), 7.01 (d, 1H), 7.23 (t, 1H), 7.32 (t, 1H), 7.46 (d, 1H)

### Example 23

### Synthesis of BET 23

1.52 g (8.00 mmol) of 4-(4-methyl-1-piperazinyl) aniline, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 23>

δ: 0.89 (t, 6H), 1.26-1.40 (m, 40H), 1.63-1.66 (m, 4H), 2.02 (dt, 8H), 2.10 (t, 2H), 2.21 (s, 3H), 2.35 (t, 4H), 2.57 (t, 2H), 3.44 (t, 4H), 5.34 (m, 4H), 6.16 (s, 1H), 6.59 (d, 2H), 6.85 (d, 2H), 7.10 (dd, 1H), 7.22 (dd, 1H), 7.29-7.37 (m, 6H), 7.44 (dd, 1H)

### Example 24

### Synthesis of BET 24

1.14 g (8.00 mmol) of 2-(4-methylpiperazin-1-yl)ethan-1-amine, 0.93 g (8.80 mmol) of benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.96 g of the target product.

<¹H-NMR (600 MHz, CDCl₃) of Compound BET 24>

δ: 0.83-0.92 (m, 6H), 1.28-1.40 (m, 40H), 1.41-1.78 (m, 5H), 1.86 (dt, 0.5H), 1.95-2.52 (m, 23.5), 3.30-3.43 (m, 1H), 3.44-3.52 (m, 0.5H), 3.60-3.68 (m, 0.5H), 5.29-5.39 (m, 4H), 6.29 (s, 0.5H), 7.05-7.19 (m, 4.5H), 7.21-7.39 (m, 5H)

### Example 25

### Synthesis of BET 25

0.81 g (8.00 mmol) of hexylamine, 1.8 g (8.80 mmol) of 4-(4-methylpiperazinyl)benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 25>

δ: 0.70-1.13 (m, 13H), 1.18-1.41 (m, 42H), 1.46-1.76 (m, 4H), 1.92-2.08 (m, 8H), 2.11-2.45 (m, 8H), 2.55-2.66 (m, 4H), 3.10-3.29 (m, 6H), 3.34-3.44 (m, 1H), 5.7-5.41 (m, 4H), 6.21 (s, 0.5H), 6.83 (d, 2H), 6.95 (d, 2H), 7.05-7.25 (m, 3.5H), 7.38 (m, 1H)

### Example 26

### Synthesis of BET 26

0.81 g (8.00 mmol) of hexylamine, 0.93 g (8.80 mmol) of benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 26>

δ: 0.89 (t, 9H), 1.26-1.40 (m, 46H), 1.53 (m, 4H), 1.66 (m, 2H), 2.02-2.05 (m, 10H), 2.25 (s, 6H), 2.57 (t, 2H), 2.67-2.69 (m, 4H), 3.18 (t, 2H), 5.34 (m, 4H), 6.16 (s, 1H), 6.98 (s, 1H), 7.10 (dd, 2H), 7.18-7.22 (m, 3H), 7.29 (ddd, 2H)

### Example 27

### Synthesis of BET 27

1.14 g (8.00 mmol) of 2-(4-methylpiperazin-1-yl)ethan-1-amine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.93 g of the target product.

<¹H-NMR (600 MHz, CDCl₃) of Compound BET 27>

δ: 0.82-0.94 (m, 9H), 1.13-1.45 (m, 50H), 1.53-1.77 (m, 5H), 1.79-1.88 (m, 2H), 1.96-2.15 (m, 3H), 2.16-2.48 (m, 13H), 2.51-2.65 (m, 2H), 3.04-3.20 (m, 2H), 5.31-5.40 (m, 4H), 6.01 (s, 1H), 7.00 (s, 1H), 7.20-7.29 (m, 1H), 7.29-7.36 (m, 1H), 7.40-7.47 (m, 1H)

### Example 28

### Synthesis of BET 28

0.92 g (8.00 mmol) of 4-heptylamine, 1.8 g (8.80 mmol) of 4-(4-methylpiperazinyl)benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 28>

δ: 0.89-0.92 (m, 12H), 1.26-1.53 (m, 50H), 1.66 (m, 2H), 2.02-2.05 (m, 10H), 2.21 (s, 3H), 2.35 (t, 4H), 2.57 (t, 2H), 3.40-3.44 (m, 5H), 5.34 (m, 4H), 6.16 (s, 1H), 6.71 (d, 2H), 7.07-7.10 (m, 3H), 7.30-7.35 (m, 2H), 7.44 (dd, 1H)

### Example 29

### Synthesis of BET 29

0.79 g (8.00 mmol) of cyclohexylamine, 1.8 g (8.80 mmol) of 4-(4-methylpiperazinyl)benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 29>

δ: 0.89 (t, 6H), 1.11 (m, 2H), 1.21-1.49 (m, 48H), 1.66 (m, 2H), 1.74 (m, 2H), 2.02-2.05 (m, 10H), 2.21 (s, 3H), 2.35 (t, 4H), 2.57 (t, 2H), 3.44 (t, 4H), 3.54 (m, 1H), 5.34 (m, 4H), 6.16 (s, 1H), 6.71 (d, 2H), 7.07-7.10 (m, 3H), 7.30-7.35 (m, 2H), 7.44 (dd, 1H)

### Example 30

### Synthesis of BET 30

0.92 g (8.00 mmol) of 4-heptylamine, 0.76 g (8.80 mmol) of valeraldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 30>

δ: 0.80-0.95 (m, 15H), 1.13-1.45 (m, 54H), 1.53-1.77 (m, 4H), 1.79-1.88 (m, 2H), 2.16-2.48 (m, 18H), 2.51-2.65 (m, 2H), 3.52-3.56 (m, 1H), 4.76-4, 80 (m, 1H), 5.31-5.40 (m, 4H), 6.98 (s, 1H), 7.18 (d, 1H), 7.25 (d, 1H)

### Example 31

### Synthesis of BET 31

0.79 g (8.00 mmol) of cyclohexylamine, 0.76 g (8.80 mmol) of valeraldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.96 g of the target product.

<¹H-NMR (600 MHz, CDCl₃) of Compound BET 31>

δ: 0.89-0.91 (t, 9H), 1.11-1.53 (m, 54H), 1.66-1.74 (m, 4H), 1.82 (m, 2H), 2.02-2.05 (m, 10H), 2.25 (s, 6H), 2.57 (t, 2H), 2.67-2.69 (m, 4H), 3.54 (m, 1H), 4.78 (t, 1H), 5.34 (m, 4H), 6.98 (s, 1H), 7.15-7.18 (m, 2H)

### Example 32

### Synthesis of BET 32

0.79 g (8.00 mmol) of cyclohexylamine, 1.2 g (8.80 mmol) of 4-methoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 32>

δ: 0.89 (t, 6H), 1.11 (m, 2H), 1.21-1.53 (m, 48H), 1.66 (m, 2H), 1.74 (m, 2H), 2.02-2.05 (m, 10H), 2.25 (s, 6H), 2.57 (t, 2H), 2.67-2.69 (m, 4H), 3.54 (m, 1H), 3.80 (s, 3H), 5.34 (m, 4H), 6.16 (s, 1H), 6.89 (d, 2H), 6.98 (s, 1H), 7.15-7.21 (m, 4H)

### Example 33

### Synthesis of BET 33

0.79 g (8.00 mmol) of cyclohexylamine, 1.62 g (8.80 mmol) of dodecanal, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 33>

δ: 0.82-0.94 (m, 9H), 1.13-1.45 (m, 68H), 1.53-1.77 (m, 6H), 1.79-1.88 (m, 2H), 2.16-2.48 (m, 18H), 2.51-2.65 (m, 2H), 3.52-3.56 (m, 1H), 4.76-4, 80 (m, 1H), 5.31-5.40 (m, 4H), 6.98 (s, 1H), 7.18 (d, 1H), 7.25 (d, 1H)

### Example 34

### Synthesis of BET 34

1.14 g (8.00 mmol) of 2-(4-methylpiperazin-1-yl)ethan-1-amine, 1.2 g (8.80 mmol) of 4-methoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 34>

δ: 0.84-0.92 (m, 6H), 1.19-1.40 (m, 43H), 1.43-1.76 (m, 5H), 1.83-1.90 (m, 0.5H), 1.95-2.49 (m, 20.5H), 3.31 (dt, 0.5H), 3.36-3.48 (m, 1H), 3.65 (dt, 0.5H), 3.78 (d, 3H), 5.29-5.40 (m, 4H), 0.5 (s, 0.5H), 6.82 (t, 2H), 7.00 (t, 2H), 7.09 (dd, 2H), 7.13-7.19 (m, 1H), 7.23 (t, 0.5H), 7.34 (dt, 1H)

### Example 35

### Synthesis of BET 35

1.14 g (8.00 mmol) of 2-(4-methylpiperazin-1-yl)ethan-1-amine, 1.62 g (8.80 mmol) of dodecanal, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 35>

δ: 0.89 (t, 9H), 1.25-1.40 (m, 58H), 1.53 (m, 2H), 1.66 (m, 2H), 1.82 (dt, 2H), 2.02-2.05 (m, 10H), 2.14 (s, 3H), 2.29 (m, 8H), 2.57 (t, 2H), 2.62 (t, 2H), 3.30 (t, 2H), 4.78 (t, 1H), 5.34 (m, 4H), 7.10 (dd, 1H), 7.22-7.25 (m, 2H), 7.30 (m, 1H)

### Example 36

### Synthesis of BET 36

0.91 g (8.00 mmol) of 1-(2-aminoethyl) pyrrolidine, 1.2 g (8.80 mmol) of 4-methoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.96 g of the target product.

<¹H-NMR (600 MHz, CDCl₃) of Compound BET 36>

δ: 0.85-0.92 (m, 6H), 1.20-1.40 (m, 40H), 1.45-1.57 (m, 2H), 1.58-1.67 (m, 4H), 1.68-1.80 (m, 1.5H), 1.82-1.89 (m, 0.5H), 1.92-2.02 (m, 9H), 2.10-2.51 (m, 9H), 3.33-3.53 (m, 1.5H), 3.63-3.70 (m, 0.5H), 3.74-3.83 (m, 3H), 5.29-5.40 (m, 4H), 6.23 (s, 0.5H), 6.83 (dd, 2H), 7.00 (dd, 2H), 7.09-7.12 (m, 1.5H), 7.14-7.20 (1.5H), 7.25 (t, 0.5H), 7.34 (dt, 1H)

### Example 37

### Synthesis of BET 37

0.91 g (8.00 mmol) of 1-(2-aminoethyl) pyrrolidine, 1.62 g (8.80 mmol) of dodecanal, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 37>

δ: 0.89 (t, 9H), 1.26-1.40 (m, 58H), 1.53 (m, 2H), 1.66-1.68 (m, 6H), 1.82 (m, 2H), 2.02-2.06 (m, 10H), 2.51-2.62 (m, 8H), 3.30 (t, 2H), 4.78 (t, 1H), 5.34 (m, 4H), 7.10 (dd, 1H), 7.22-7.30 (m, 3H)

### Example 38

### Synthesis of BET 38

0.81 g (8.00 mmol) of hexylamine, 1.2 g (8.80 mmol) of 4-methoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 38>

δ: 0.89 (t, 9H), 1.25-1.40 (m, 46H), 1.53 (m, 4H), 1.66 (m, 2H), 2.02-2.05 (m, 10H), 2.25 (s, 6H), 2.57 (t, 2H), 2.67-2.69 (m, 4H), 3.18 (t, 2H), 3.80 (s, 3H), 5.34 (m, 4H), 6.16 (s, 1H), 6.89 (d, 2H), 6.98 (s, 1H), 7.15-7.21 (m, 4H)

### Example 39

### Synthesis of BET 39

0.92 g (8.00 mmol) of 4-heptylamine, 0.93 g (8.80 mmol) of benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 39>

δ: 0.81-0.94 (m, 12H), 1.13-1.70 (m, 50H), 1.79-1.85 (m, 2H), 2.16-2.50 (m, 18H), 2.53-2.65 (m, 4H), 3.48-3.53 (m, 1H), 5.31-5.40 (m, 4H), 6.29 (s, 0.5H), 6.99-7.19 (m, 3.5H), 7.21-7.39 (m, 5H)

### Example 40

### Synthesis of BET 40

1.48 g (8.00 mmol) of dodecylamine, 0.93 g (8.80 mmol) of benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 40>

δ: 0.89 (t, 9H), 1.26-1.40 (m, 58H), 1.53 (m, 4H), 1.66 (m, 2H), 2.02-2.05 (m, 10H), 2.25 (s, 6H), 2.57 (t, 2H), 2.67-2.69 (m, 4H), 3.18 (t, 2H), 5.34 (m, 4H), 6.16 (s, 1H), 6.98 (s, 1H), 7.10 (m, 2H), 7.15 (d, 1H), 7.18 (d, 1H), 7.22-7.29 (3H)

### Example 41

### Synthesis of BET 41

0.92 g (8.00 mmol) of heptylamine, 1.62 g (8.80 mmol) of dodecanal, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.09 g of the target product.

<¹H-NMR (600 MHz, CDCl₃) of Compound BET 41>8:0.89 (t, 12H), 1.25-1.40 (m, 66H), 1.53 (m, 4H), 1.66 (m, 2H), 1.82 (dt, 2H), 2.02-2.05 (m, 10H), 2.25 (s, 6H), 2.57 (t, 2H), 2.67-2.69 (m, 4H), 3.18 (t, 2H), 4.78 (t, 1H), 5, 34 (m, 4H), 6.98 (s, 1H), 7.15 (dd, 1H), 7.18 (d, 1H)

### Example 42

### Synthesis of BET 42

0.71 g (8.00 mmol) of 3-methoxypropylamine, 0.93 g (8.80 mmol) of benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 42>

δ: 0.83-0.93 (m, 6H), 1.12-1.68 (m, 45H), 1.79-1.85 (m, 2H), 2.16-2.50 (m, 16H), 2.53-2.65 (m, 6H), 3.35 (t, 2H), 3.42 (s, 3H), 3.48-3.53 (m, 1H), 5.31-5.40 (m, 4H), 6.29 (s, 0.5H), 6.99-7.19 (m, 3.5H), 7.21-7.39 (m, 5H)

### Example 43

### Synthesis of BET 43

0.91 g (8.00 mmol) of 1-(2-aminoethyl) pyrrolidine, 0.88 g (8.80 mmol) of 2-ethylbutyraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.92 g of the target product.

<¹H-NMR (600 MHz, CDCl₃) of Compound BET 43>

δ: 0.86-0.89 (t, 12H), 1.26-1.33 (m, 40H), 1.53-1.55 (m, 6H), 1.66-1.68 (m, 6H), 2.02-2.05 (m, 10H), 2.30 (m, 1H), 2.51 (m, 4H), 2.57-2.62 (m, 4H), 3.30 (t, 2H), 4.68 (t, 1H), 5.34 (m, 4H), 7.10 (dd, 1H), 7.22-7.30 (m, 3H)

### Example 44

### Synthesis of BET 44

0.91 g (8.00 mmol) of 1-(2-aminoethyl) pyrrolidine, 0.93 g (8.80 mmol) of benzaldehyde, and 1.99 g (16.0 mmol) of 3-methoxyphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.96 g of the target product.

<¹H-NMR (600 MHz, CDCl₃) of Compound BET 44>

δ: 0.89 (t, 6H), 1.26-1.40 (m, 40H), 1.53 (m, 2H), 1.66-1.68 (m, 6H), 2.02-2.05 (m, 10H), 2.51 (t, 4H), 2.57 (t, 2H), 2.62 (t, 2H), 3.30 (t, 2H), 3.72 (s, 3H), 5.34 (m, 4H), 6.16 (s, 1H), 6.78 (dd, 1H), 6.88 (dd, 1H), 7.10 (dd, 2H), 7.22 (dd, 1H), 7.29 (m, 2H), 7.36 (dd, 1H)

### Example 45

### Synthesis of BET 45

0.91 g (8.00 mmol) of 1-(2-aminoethyl) pyrrolidine, 1.46 g (8.80 mmol) of 3,4-dimethoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 45>

δ: 0.83-0.93 (m, 6H), 1.18-1.42 (m, 42H), 1.48-1.79 (m, 6H), 1.94-2.10 (m, 9H), 2.16-2.53 (m, 9H), 3.38-3.61 (m, 2H), 3.77 (d, 3H), 3.86 (d, 3H), 5.29-5.40 (m, 4H), 6.25 (s, 0.5H), 6.56-6.67 (m, 2H), 6.80 (t, 1H), 7.07-7.41 (m, 4.5H)

### Example 46

### Synthesis of BET 46

0.91 g (8.00 mmol) of 1-(2-aminoethyl) pyrrolidine, 1.31 g (8.80 mmol) of p-dimethylaminobenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.97 g of the target product.

<¹H-NMR (600 MHz, CDCl₃) of Compound BET 46>

δ: 0.89 (t, 6H), 1.26-1.40 (m, 40H), 1.53 (m, 2H), 1.66-1.68 (m, 6H), 2.02-2.05 (m, 10H), 2.51-2.62 (m, 8H), 2.94 (s, 6H), 3.30 (t, 2H), 5.34 (m, 4H), 6.16 (s, 1H), 6.71 (d, 2H), 7.07-7.10 (m, 3H), 7.30-7.35 (m, 2H), 7.44 (dd, 1H)

### Example 47

### Synthesis of BET 47

0.91 g (8.00 mmol) of 1-(2-aminoethyl) pyrrolidine, 0.94 g (8.80 mmol) of 2-pyridinecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.93 g of the target product.

<¹H-NMR (600 MHz, CDCl₃) of Compound BET 47>

δ: 0.89 (t, 6H), 1.26-1.40 (m, 40H), 1.53 (m, 2H), 1.66-1.68 (m, 6H), 2.02-2.05 (m, 10H), 2.51 (t, 4H), 2.57 (t, 2H), 2.62 (t, 2H), 3.30 (t, 2H), 5.34 (m, 4H), 6.16 (s, 1H), 7.10 (dd, 1H), 7.16 (m, 1H), 7.24 (m, 1H), 7.30-7.35 (m, 2H), 7.44 (dd, 1H), 7.50 (m, 1H), 8.54 (dd, 1H)

### Example 48

### Synthesis of BET 48

0.91 g (8.00 mmol) of 1-(2-aminoethyl) pyrrolidine, 1.69 g (8.80 mmol) of 4-amyloxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 48>

δ: 0.79 (m, 9H), 1.14-1.82 (m, 56H), 1.91-2.07 (m, 8H), 2.10-2.52 (m, 8H), 3.29-3.53 (m, 1.5H), 3.61-3.71 (m, 0.5H), 3.87-3.98 (m, 2H), 5.27-5.30 (m, 4H), 6.24 (s, 0.5H), 6.82 (t, 2H), 6.98 (t, 2H), 7.06-7.20 (m, 3.5H), 7.21-7.38 (m, 1H)

### Example 49

### Synthesis of BET 49

1.21 g (8.00 mmol) of 2-(4-methoxyphenyl)ethylamine, 1.2 g (8.80 mmol) of 4-methoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 49>

δ: 0.89 (t, 6H), 1.26-1.40 (m, 40H), 1.53 (m, 2H), 1.66 (m, 2H), 2.02-2.05 (m, 10H), 2.25 (s, 6H), 2.57 (t, 2H), 2.67-2.69 (m, 4H), 2.81 (t, 2H), 3.53 (t, 2H), 3.80 (s, 6H), 5.34 (m, 4H), 6.16 (s, 1H), 6.86-6.89 (m, 4H), 6.98 (s, 1H), 7.13-7.25 (m, 6H)

### Example 50

### Synthesis of BET 50

1.21 g (8.00 mmol) of 2-(4-methoxyphenyl)ethylamine, 1.69 g (8.80 mmol) of 4-amyloxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.14 g of the target product.

<¹H-NMR (600 MHz, CDCl₃) of Compound BET 50>

δ: 0.89 (t, 9H), 1.26-1.40 (m, 44H), 1.53 (m, 2H), 1.66 (m, 2H), 1.77 (m, 2H), 2.02-2.05 (m, 10H), 2.25 (s, 6H), 2.57 (t, 2H), 2.67-2.69 (m, 4H), 2.81 (t, 2H), 3.53 (t, 2H), 3.80 (s, 3H), 4.11 (t, 2H), 5.34 (m, 4H), 6.16 (s, 1H), 6.86-6.89 (m, 4H), 6.98 (s, 1H), 7.13-7.21 (m, 6H)

### Example 51

### Synthesis of BET 51

0.81 g (8.00 mmol) of hexylamine, 1.69 g (8.80 mmol) of 4-amyloxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.09 g of the target product.

<¹H-NMR (600 MHz, CDCl₃) of Compound BET 51>

δ: 0.71-0.83 (m, 4H), 0.85-1.14 (m, 16H), 1.20-1.66 (m, 47H), 1.68-1.83 (m, 3H), 1.95-2.08 (m, 8H), 2.10-2.17 (m, 0.5H), 2.19-2.52 (m, 11.5H), 2.66-2.77 (m, 2H), 3.10-3.27 (m, 1.5H), 3.35-3.43 (m, 0.5H), 3.88-3.96 (m, 2H), 5.27-5.39 (m, 4H), 6.20 (s, 0.5H), 6.81 (t, 2H), 6.89-7.04 (m, 4H), 7.12-7.21 (m, 1.5H)

### Example 52

### Synthesis of BET 52

0.68 g (8.00 mmol) of piperidine, 1.32 g (8.80 mmol) of 4-hydroxy-3,5-dimethylbenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.94 g of the target product.

<¹H-NMR (600 MHz, CDCl₃) of Compound BET 52>

δ: 0.89 (t, 6H), 1.26-1.49 (m, 46H), 1.66 (m, 4H), 2.02 (m, 8H), 2.15 (s, 6H), 2.32 (t, 4H), 2.57 (t, 4H), 5.14 (s, 1H), 5.34 (m, 4H), 6.84 (s, 2H), 7.10 (dd, 1H), 7.30-7.35 (m, 2H), 7.44 (dd, 1H)

### Example 53

### Synthesis of BET 53

1.21 g (8.00 mmol) of 2-(4-methoxyphenyl)ethylamine, 1.8 g (8.80 mmol) of 4-(4-methylpiperazinyl)benzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 53>

δ: 0.89 (t, 6H), 1.26-1.40 (m, 40H), 1.53 (m, 2H), 1.66 (m, 2H), 2.02-2.05 (m, 10H), 2.21 (s, 3H), 2.35 (t, 4H), 2.57 (t, 2H), 2.81 (t, 2H), 3.44 (t, 4H), 3.53 (t, 2H), 3.80 (s, 3H), 5.34 (m, 4H), 6.16 (s, 1H), 6.71 (d, 2H), 6.86 (d, 2H), 7.07-7.13 (m, 5H), 7.30-7.35 (m, 2H), 7.44 (dd, 1H)

### Example 54

### Synthesis of BET 54

1.14 g (8.00 mmol) of 2-(4-methylpiperazin-1-yl)ethan-1-amine, 1.43 g (8.80 mmol) of 4-butylbenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 54>

δ: 0.85-0.96 (m, 9H), 1.17-1.39 (m, 43H), 1.40-1.76 (m, 8H), 1.95-2.50 (m, 22H), 2.58 (dd, 2H), 3.30 (dt, 0.5H), 3.36-3.49 (m, 1H), 3.67 (dt, 0.5H), 5.28-5.39 (m, 4H), 6.25 (s, 0.5H), 6.98 (dd, 2H), 7.09 (dd, 3.5H), 7.14-7.18 (m, 1H), 7.20-7.24 (m, 1H), 7.34 (dt, 1H)

### Example 55

### Synthesis of BET 55

1.14 g (8.00 mmol) of 2-(4-methylpiperazin-1-yl)ethan-1-amine, 1.69 g (8.80 mmol) of 4-amyloxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 55>

δ: 0.89-0.90 (m, 9H), 1.26-1.40 (m, 44H), 1.53 (m, 2H), 1.66 (m, 2H), 1.77 (m, 2H), 2.02-2.05 (m, 10H), 2.14 (s, 3H), 2.29 (m, 8H), 2.57-2.62 (m, 4H), 3.30 (t, 2H), 4.11 (t, 2H), 5.34 (m, 4H), 6.16 (s, 1H), 6.89 (d, 2H), 7.10 (dd, 1H), 7.21 (d, 2H), 7.30-7.35 (m, 2H), 7.44 (dd, 1H)

### Example 56

### Synthesis of BET 56

1.14 g (8.00 mmol) of 2-(4-methylpiperazin-1-yl)ethan-1-amine, 0.94 g (8.80 mmol) of 2-pyridinecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.96 g of the target product.

<¹H-NMR (600 MHz, CDCl₃) of Compound BET 56>

δ: 0.89 (t, 6H), 1.26-1.40 (m, 40H), 1.53 (m, 2H), 1.66 (m, 2H), 2.02-2.05 (m, 10H), 2.14 (s, 3H), 2.29 (m, 8H), 2.57 (t, 2H), 2.62 (t, 2H), 3.30 (t, 2H), 5.34 (m, 4H), 6.16 (s, 1H), 7.10 (dd, 1H), 7.16 (m, 1H), 7.24 (d, 1H), 7.30-7.35 (m, 2H), 7.44 (d, 1H), 7.50 (m, 1H), 8.54 (dd, 1H)

### Example 57

### Synthesis of BET 57

1.14 g (8.00 mmol) of 2-(4-methylpiperazin-1-yl)ethan-1-amine, 1.46 g (8.80 mmol) of 3,4-dimethoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 57>

δ: 0.84-0.93 (m, 6H), 1.20-1.40 (m, 40H), 1.48-1.65 (m, 4H), 1.65-1.68 (m, 1H), 1.84-2.06 (m, 10H), 2.14-2.52 (m, 14H), 3.36-3.58 (m, 2H), 3.76 (d, 3H), 3.86 (d, 3H), 5.28-5.39 (m, 4H), 6.24 (s, 0.5H), 6.56 (d, 1H), 6.63 (dd, 1H), 6.79 (dd, 1H), 7.06-7.20 (m, 3H), 7.22 (t, 0.5H), 7.35 (dt, 1H)

### Example 58

### Synthesis of BET 58

1.21 g (8.00 mmol) of 2-(4-methoxyphenyl)ethylamine, 1.43 g (8.80 mmol) of 4-butylbenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.03 g of the target product.

<¹H-NMR (600 MHz, CDCl₃) of Compound BET 58>

δ: 0.89-0.90 (m, 9H), 1.26-1.40 (m, 42H), 1.53-1.56 (m, 4H), 1.66 (m, 2H), 2.02-2.05 (m, 10H), 2.57 (t, 2H), 2.65 (t, 2H), 2.81 (t, 2H), 3.53 (t, 2H), 3.80 (s, 3H), 5.34 (m, 4H), 6.16 (s, 1H), 6.86 (d, 2H), 7.08-7.13 (m, 5H), 7.29-7.35 (m, 4H), 7.44 (dd, 1H)

### Example 59

### Synthesis of BET 59

1.26 g (8.00 mmol) of 2-(furfurylthio)ethylamine, 1.43 g (8.80 mmol) of 4-butylbenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.12 g of the target product.

<¹H-NMR (600 MHz, CDCl₃) of Compound BET 59>

δ: 0.89 (t, 9H), 1.26-1.40 (m, 42H), 1.53-1.56 (m, 4H), 1.66 (m, 2H), 2.02-2.05 (m, 10H), 2.25 (s, 6H), 2.57 (t, 2H), 2.65-2.70 (m, 8H), 3.57 (t, 2H), 3.73 (s, 2H), 5.34 (m, 4H), 5.94 (d, 1H), 6.16 (s, 1H), 6.23 (dd, 1H), 6.98 (s, 1H), 7.08 (d, 2H), 7.15-7.18 (d, 2H), 7.27-7.29 (m, 3H)

### Example 60

### Synthesis of BET 60

1.26 g (8.00 mmol) of 2-(furfurylthio)ethylamine, 0.93 g (8.80 mmol) of benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 60>

δ: 0.81-0.92 (m, 6H), 1.17-1.39 (m, 40H), 1.41-1.86 (m, 6H), 1.95-2.51 (m, 21H), 2.70 (dt, 2H), 3.35-3.54 (m, 3.5H), 3.67-3.76 (m, 0.5H), 5.28-5.40 (m, 4H), 6.02-6.29 (m, 2.5H), 6.90 (d, 1H), 6.97-7.11 (m, 2.5H), 7.15-7.35 (m, 5H)

### Example 61

### Synthesis of BET 61

1.26 g (8.00 mmol) of 2-(furfurylthio)ethylamine, 1.2 g (8.80 mmol) of 4-methoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.09 g of the target product.

<¹H-NMR (600 MHz, CDCl₃) of Compound BET 61>

δ: 0.89 (t, 6H), 1.26-1.40 (m, 40H), 1.53 (m, 2H), 1.66 (m, 2H), 2.02-2.05 (m, 10H), 2.25 (s, 6H), 2.57 (t, 2H), 2.67-2.70 (m, 6H), 3.57 (t, 2H), 3.73 (s, 2H), 3.80 (s, 3H), 5.34 (m, 4H), 5.94 (d, 1H), 6.16 (s, 1H), 6.23 (m, 1H), 6.89 (d, 2H), 6.98 (s, 1H), 7.15-7.21 (m, 4H), 7.27 (d, 1H)

### Example 62

### Synthesis of BET 62

1.0 g (8.00 mmol) of cis-octahydroisoindole, 0.93 g (8.80 mmol) of benzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.0 g of the target product.

<¹H-NMR (600 MHz, CDCl₃) of Compound BET 62>

δ: 0.89 (t, 6H), 1.26-1.66 (m, 54H), 2.02 (dt, 8H), 2.17 (m, 2H), 2.32 (t, 2H), 2.42 (m, 2H), 2.57 (t, 2H), 3.83 (s, 3H), 5.14 (s, 1H), 5.20 (s, 2H), 5.34 (m, 4H), 6.74 (s, 1H), 6.94 (s, 1H), 7.22 (m, 3H), 7.54 (dd, 2H)

### Example 63

### Synthesis of BET 63

0.92 g (8.00 mmol) of N-methylhexan-1-amine, 1.46 g (8.80 mmol) of 3,4-dimethoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.06 g of the target product.

<¹H-NMR (600 MHz, CDCl₃) of Compound BET 63>

δ: 0.81-0.92 (m, 6H), 1.17-1.39 (m, 54H), 1.41-1.86 (m, 7H), 1.95-2.30 (m, 13H), 2.40-2.45 (m, 2H), 2.57 (t, 2H), 3.75 (s, 3H), 3.83 (s, 3H), 5.28-5.40 (m, 4H), 6.74 (s, 1H), 6.86-6.91 (m, 2H), 6.97 (s, 1H), 7.00 (s, 1H)

### Example 64

### Synthesis of BET 64

0.91 g (8.00 mmol) of 4-amino-1-methylpiperidine, 1.41 g (8.80 mmol) of 4-(propargyloxy)benzaldehyde, and 1.95 g (16.0 mmol) of 4-ethylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 64>

δ: 0.89 (t, 6H), 1.18-1.40 (m, 43H), 1.53-1.66 (m, 6H), 1.84 (m, 2H), 2.02-2.05 (m, 10H), 2.18 (s, 3H), 2.41-2.51 (m, 4H), 2.57 (t, 2H), 2.72 (q, 2H), 3.37 (s, 1H), 3.60 (m, 1H), 4.68 (s, 2H), 5.34 (m, 4H), 6.16 (m, 1H), 6.89 (d, 2H), 6.98 (s, 1H), 7.15-7.21 (m, 4H)

### Example 65

### Synthesis of BET 65

0.91 g (8.00 mmol) of 3-methylcyclohexylamine (cis- and trans-mixture), 1.74 g (8.80 mmol) of 4-phenoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.11 g of the target product.

<¹H-NMR (600 MHz, CDCl₃) of Compound BET 65>

δ: 0.86-0.89 (m, 9H), 1.26-1.74 (m, 53H), 2.02-2.05 (m, 10H), 2.25 (s, 6H), 2.57 (t, 2H), 2.67-2.69 (m, 4H), 3.54 (m, 1H), 5.34 (m, 4H), 6.16 (s, 1H), 6.98-7.01 (m, 3H), 7.09 (dd, 1H), 7.15-7.18 (m, 4H), 7.32-7.36 (m, 4H)

### Example 66

### Synthesis of BET 66

0.91 g (8.00 mmol) of 4-amino-1-methylpiperidine, 1.59 g (8.80 mmol) of 3-nitro-p-anisaldehyde, and 2.47 g (16.0 mmol) of 2,5-dimethoxyphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 66>

δ: 0.81-0.92 (m, 6H), 1.17-1.39 (m, 40H), 1.41-1.86 (m, 8H), 1.95-2.30 (m, 13H), 2.40-2.55 (m, 6H), 3.54-3.57 (m, 1H), 3.72 (s, 3H), 3.83 (s, 3H), 4.02 (s, 3H), 5.28-5.40 (m, 4H), 6.20 (s, 0.5H), 6.90-6.95 (m, 2.5H), 7.26 (d, 1H), 7.56 (d, 1H), 7.84 (s, 1H)

### Example 67

### Synthesis of BET 67

0.98 g (8.00 mmol) of 4-(2-aminoethyl)pyridine, 1.46 g (8.80 mmol) of 2,6-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 67>

δ: 0.89 (t, 6H), 1.26-1.40 (M, 40H), 1.53 (m, 2H), 1.66 (m, 2H), 2.02-2.05 (m, 10H), 2.25 (s, 6H), 2.57 (t, 2H), 2.67-2.69 (m, 4H), 3.54-3.59 (m, 4H), 3.72 (s, 6H), 5.34 (m, 4H), 6.16 (s, 1H), 6.61 (dd, 2H), 6.98 (s, 1H), 7.08 (d, 2H), 7.15-7.18 (m, 2H), 7.36 (dd, 1H), 8.44 (d, 2H)

### Example 68

### Synthesis of BET 68

0.91 g (8.00 mmol) of 4-amino-1-methylpiperidine, 0.94 g (8.80 mmol) of 4-pyridinecarboxaldehyde, and 2.44 g (16.0 mmol) of 4-(2-methoxyethyl) phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 68>

δ: 0.89 (t, 6H), 1.26-1.40 (m, 40H), 1.53 (m, 1H), 1.59 (m, 2H), 1.66 (m, 1H), 1.84 (m, 2H), 2.02-2.05 (m, 10H), 2.18 (s, 3H), 2.41 (m, 2H), 2.57 (t, 2H), 2.51 (m, 2H), 2.85 (t, 2H), 3.40 (s, 3H), 3.52 (t, 2H), 3.60 (m, 1H), 5.34 (m, 4H), 6.16 (s, 1H), 6.98 (s, 1H), 7.03 (d, 2H), 7.15-7.18 (m, 2H), 8.43 (d, 2H)

### Example 69

### Synthesis of BET 69

1.29 g (8.00 mmol) of 4-phenylpiperidine, 1.6 g (8.80 mmol) of syringaldehyde, and 2.63 g (16.0 mmol) of 4-tert-amylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.14 g of the target product.

<¹H-NMR (600 MHz, CDCl₃) of Compound BET 69>8:0.80-0.95 (m, 15H), 1.17-1.44 (m, 54H), 1.65-1.74 (m, 2H), 1.95-2.10 (m, 4H), 2.40-2.60 (m, 7H), 3.83 (s, 6H), 5.28-5.40 (m, 4H), 6.20 (s, 0.5H), 6.65 (s, 2H), 7.02-7.21 (m, 2.5H), 7.26-7.35 (m, 5H), 7.51 (s, 1H)

### Example 70

### Synthesis of BET 70

0.92 g (8.00 mmol) of N-methylhexan-1-amine, 0.93 g (8.80 mmol) of benzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 70>

δ: 0.89 (t, 9H), 1.26-1.40 (m, 48H), 1.66 (m, 4H), 2.02 (m, 8H), 2.22 (s, 3H), 2.32 (t, 2H), 2.43 (t, 2H), 2.57 (t, 2H), 3.83 (s, 3H), 5.14 (s, 1H), 5.20 (d, 2H), 5.34 (m, 4H), 6.74 (s, 1H), 6.94 (s, 1H), 7.22-7.23 (m, 3H), 7.54 (dd, 2H)

### Example 71

### Synthesis of BET 71

0.59 g (8.00 mmol) of N-methylpropylamine, 0.93 g (8.80 mmol) of benzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.95 g of the target product.

<¹H-NMR (600 MHz, CDCl₃) of Compound BET 71>

δ: 0.89-0.91 (m, 9H), 1.26-1.43 (m, 42H), 1.66 (m, 4H), 2.02 (dt, 8H), 2.22 (s, 3H), 2.32 (t, 2H), 2.43 (t, 2H), 2.57 (t, 2H), 3.83 (s, 3H), 5.14 (s, 1H), 5.20 (s, 2H), 5.34 (m, 4H), 6.74 (s, 1H), 6.94 (s, 1H), 7.22-7.23 (m, 3H), 7.54 (dd, 2H)

### Example 72

### Synthesis of BET 72

0.59 g (8.00 mmol) of N-methylpropylamine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 72>

δ: 0.89-0.91 (m, 9H), 1.26-1.43 (m, 42H), 1.66 (m, 4H), 2.02 (dt, 8H), 2.22 (s, 3H), 2.32 (t, 2H), 2.43 (t, 2H), 2.57 (t, 2H), 3.80 (s, 3H), 3.83 (s, 3H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 73

### Synthesis of BET 73

0.68 g (8.00 mmol) of piperidine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 73>

δ: 0.89 (t, 6H), 1.26-1.40 (m, 42H), 1.49 (m, 4H), 1.66 (m, 4H), 2.02 (dt, 8H), 2.32 (m, 6H), 2.57 (t, 2H), 3.80 (s, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 74

### Synthesis of BET 74

0.57 g (8.00 mmol) of pyrrolidine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 74>

δ: 0.89 (t, 6H), 1.26-1.40 (m, 40H), 1.59 (m, 4H), 1.66 (m, 4H), 2.02 (dt, 8H), 2.35 (m, 6H), 2.57 (t, 2H), 3.80 (s, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 75

### Synthesis of BET 75

1.31 g (8.00 mmol) of 1-(methylsulfonyl) piperazine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 75>

δ: 0.89 (t, 6H), 1.26-1.40 (m, 40H), 1.66 (m, 4H), 2.02 (dt, 8H), 2.32 (m, 2H), 2.57 (t, 2H), 2.65 (m, 4H), 2.73 (m, 4H), 2.87 (s, 3H), 3.80 (s, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 76

### Synthesis of BET 76

0.82 g (8.00 mmol) of N,N,N′-trimethylethylenediamine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 76>

δ: 0.89 (t, 6H), 1.26-1.40 (m, 40H), 1.66 (m, 4H), 2.02 (dt, 8H), 2.21-2.22 (s, 9H), 2.32 (t, 2H), 2.46 (m, 4H), 2.57 (t, 2H), 3.80-3.83 (m, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 77

### Synthesis of BET 77

1.23 g (8.00 mmol) of 4-(1-pyrrolidinyl) piperidine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 77>

δ: 0.89 (t, 6H), 1.26-1.44 (m, 44H), 1.66-1.68 (m, 12H), 2.02 (dt, 8H), 2.32 (t, 2H), 2.41-1.57 (m, 6H), 2.63 (m, 1H), 3.80-3.83 (m, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 78

### Synthesis of BET 78

0.91 g (8.00 mmol) of 3-(dimethylamino)pyrrolidine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.03 g of the target product.

<¹H-NMR (600 MHz, CDCl₃) of Compound BET 78>

δ: 0.89 (t, 6H), 1.26-1.33 (m, 40H), 1.52-1.66 (m, 8H), 2.02 (dt, 8H), 2.22 (s, 6H), 2.29-1.32 (m, 6H), 2.72 (m, 1H), 3.80-3.83 (m, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 79

### Synthesis of BET 79

1.14 g (8.00 mmol) of 3-(diethylamino)pyrrolidine, 1.2 g (8.80 mmol) of 4-methoxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.06 g of the target product.

<¹H-NMR (600 MHz, CDCl₃) of Compound BET 79>

δ: 0.89 (t, 6H), 1.03 (q, 6H), 1.26-1.33 (m, 40H), 1.52-1.77 (m, 8H), 2.02 (dt, 8H), 2.29-1.40 (m, 10H), 2.72 (m, 1H), 3.80-3.83 (m, 6H), 5.14 (s, 1H), 5.20 (s, 2H), 5.34 (m, 4H), 6.74 (s, 1H), 6.89 (d, 2H), 6.94 (s, 1H), 7.21 (d, 2H)

### Example 80

### Synthesis of BET 80

0.81 g (8.00 mmol) of hexylamine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.06 g of the target product.

<¹H-NMR (600 MHz, CDCl₃) of Compound BET 80>

δ: 0.89 (m, 9H), 1.26-1.40 (m, 46H), 1.53 (m, 4H), 1.66 (m, 2H), 2.02-2.05 (m, 10H), 2.25 (s, 6H), 2.57 (t, 2H), 2.67-2.69 (m, 4H), 3.18 (t, 2H), 3.75 (s, 3H), 3.83 (s, 3H), 5.34 (m, 4H), 6.16 (s, 1H), 6.87-6.89 (m, 2H), 6.97-6.98 (m, 2H), 7.15-7.18 (m, 2H)

### Example 81

### Synthesis of BET 81

1.48 g (8.00 mmol) of dodecylamine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.15 g of the target product.

<¹H-NMR (600 MHz, CDCl₃) of Compound BET 81>

δ: 0.89 (m, 9H), 1.26-1.40 (m, 58H), 1.53 (m, 4H), 1.66 (m, 2H), 2.02-2.05 (m, 10H), 2.25 (s, 6H), 2.57 (t, 2H), 2.67-2.69 (m, 4H), 3.18 (t, 2H), 3.75 (s, 3H), 3.83 (s, 3H), 5.34 (m, 4H), 6.16 (s, 1H), 6.87-6.89 (m, 2H), 6.97-6.98 (m, 2H), 7.15-7.18 (m, 2H)

### Example 82

### Synthesis of BET 82

0.91 g (8.00 mmol) of 3-methylcyclohexylamine (cis- and trans-mixture), 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 82>

δ: 0.86-0.89 (m, 9H), 1.26-1.74 (m, 53H), 2.02-2.05 (m, 10H), 2.25 (s, 6H), 2.57 (t, 2H), 2.67-2.69 (m, 4H), 3.54 (m, 1H), 3.75 (s, 3H), 3.83 (s, 3H), 5.34 (m, 4H), 6.16 (s, 1H), 6.87-6.89 (m, 2H), 6.97-6.98 (m, 2H), 7.15-7.18 (m, 2H)

### Example 83

### Synthesis of BET 83

0.92 g (8.00 mmol) of 4-heptylamine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 83>

δ: 0.89-0.92 (m, 12H), 1.26-1.40 (m, 50H), 1.66 (m, 2H), 2.02-2.05 (m, 10H), 2.25 (s, 6H), 2.57 (t, 2H), 2.67-2.69 (m, 4H), 3.40 (s, 1H), 3.75 (s, 3H), 3.83 (s, 3H), 5.34 (m, 4H), 6.16 (s, 1H), 6.87-6.89 (m, 2H), 6.97-6.98 (m, 2H), 7.15-7.18 (m, 2H)

### Example 84

### Synthesis of BET 84

0.81 g (8.00 mmol) of hexylamine, 1.31 g (8.80 mmol) of 4-dimethylaminobenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 84>

δ: 0.89 (m, 9H), 1.25-1.40 (m, 46H), 1.53 (m, 4H), 1.66 (m, 2H), 2.02-2.05 (m, 10H), 2.25 (s, 6H), 2.57 (t, 2H), 2.67-2.69 (m, 4H), 2.94 (s, 6H), 3.18 (t, 2H), 5.34 (m, 4H), 6.16 (s, 1H), 6.71 (m, 2H), 6.98 (m, 1H), 7.07 (m, 2H), 7.18 (m, 2H)

### Example 85

### Synthesis of BET 85

0.79 g (8.00 mmol) of cyclohexylamine, 1.31 g (8.80 mmol) of 4-dimethylaminobenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 85>

δ: 0.89 (m, 6H), 1.11-1.74 (m, 54H), 2.02-2.05 (m, 10H), 2.25 (s, 6H), 2.57 (t, 2H), 2.67-2.69 (m, 4H), 2.94 (s, 6H), 3.54 (m, 1H), 5.34 (m, 4H), 6.16 (s, 1H), 6.71 (m, 2H), 6.98 (m, 1H), 7.07 (m, 2H), 7.15-7.18 (m, 2H)

### Example 86

### Synthesis of BET 86

0.71 g (8.00 mmol) of 3-methoxypropylamine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 86>

δ: 0.89 (m, 6H), 1.26-1.40 (m, 40H), 1.53 (tt, 2H), 1.66 (tt, 2H), 1.72 (tt, 2H), 2.02-2.05 (m, 10H), 2.25 (s, 6H), 2.57 (t, 2H), 2.67-2.69 (m, 4H), 3.18 (t, 2H), 3.35 (t, 2H), 5.40 (s, 3H), 3.75 (s, 3H), 3.83 (s, 3H), 5.34 (m, 4H), 6.16 (s, 1H), 6.87-6.89 (m, 2H), 6.97-6.98 (m, 2H), 7.15-7.18 (m, 2H)

### Example 87

### Synthesis of BET 87

0.71 g (8.00 mmol) of 3-methoxypropylamine, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 87>

δ: 0.89 (m, 6H), 1.26-1.40 (m, 40H), 1.53 (tt, 2H), 1.66-1.72 (m, 4H), 2.02-2.05 (m, 10H), 2.25 (s, 6H), 2.57 (t, 2H), 2.67-2.69 (m, 4H), 3.18 (t, 2H), 3.35-3.40 (m, 5H), 5.34 (m, 4H), 6.16 (s, 1H), 6.98 (m, 1H), 7.15-7.18 (m, 3H), 7.24 (m, 1H), 7.50 (m, 1H), 8.54 (m, 1H)

### Example 88

### Synthesis of BET 88

1.48 g (8.00 mmol) of dodecylamine, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 88>

δ: 0.89 (m, 9H), 1.26-1.40 (m, 58H), 1.53 (tt, 2H), 1.66 (m, 4H), 2.02-2.05 (m, 10H), 2.25 (s, 6H), 2.57 (t, 2H), 2.67-2.69 (m, 4H), 3.18 (t, 2H), 5.34 (m, 4H), 6.16 (s, 1H), 6.98 (m, 1H), 7.15-7.18 (m, 3H), 7.24 (m, 1H), 7.50 (m, 1H), 8.54 (m, 1H)

### Example 89

### Synthesis of BET 89

1.21 g (8.00 mmol) of 2-(benzyloxy) ethanamine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.11 g of the target product.

<¹H-NMR (600 MHz, CDCl₃) of Compound BET 89>

δ: 0.89 (m, 6H), 1.26-1.40 (m, 40H), 1.53 (tt, 2H), 1.66 (m, 2H), 2.02-2.05 (m, 10H), 2.25 (s, 6H), 2.57 (t, 2H), 2.67-2.69 (m, 4H), 3.37 (t, 2H), 3.63 (t, 2H), 3.75 (s, 3H), 3.83 (s, 3H), 4.80 (s, 2H), 5.34 (m, 4H), 6.16 (s, 1H), 6.87-6.89 (m, 2H), 6.97-6.98 (m, 2H), 7.15-7.18 (m, 2H), 7.31-7.32 (m, 5H)

### Example 90

### Synthesis of BET 90

1.21 g (8.00 mmol) of 2-(benzyloxy) ethanamine, 0.93 g (8.80 mmol) of benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 90>

δ: 0.89 (m, 6H), 1.26-1.40 (m, 40H), 1.53 (tt, 2H), 1.66 (m, 2H), 2.02-2.05 (m, 10H), 2.25 (s, 6H), 2.57 (t, 2H), 2.67-2.69 (m, 4H), 3.37 (t, 2H), 3.63 (t, 2H), 4.80 (s, 2H), 5.34 (m, 4H), 6.16 (s, 1H), 6.87-6.89 (m, 2H), 6.98 (m, 1H), 7.10 (m, 2H), 7.15-7.22 (m, 3H), 7.31-7.32 (m, 5H)

### Example 91

### Synthesis of BET 91

1.21 g (8.00 mmol) of 2-(benzyloxy)ethanamine, 0.94 g (8.80 mmol) of 4-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 91>

δ: 0.89 (m, 6H), 1.26-1.40 (m, 40H), 1.53 (tt, 2H), 1.66 (m, 2H), 2.02-2.05 (m, 10H), 2.25 (s, 6H), 2.57 (t, 2H), 2.67-2.69 (m, 4H), 3.37 (t, 2H), 3.63 (t, 2H), 4.80 (s, 2H), 5.34 (m, 4H), 6.16 (s, 1H), 6.98 (m, 1H), 7.03 (m, 2H), 7.15-7.18 (m, 2H), 7.31-7.32 (m, 5H), 8.43 (m, 2H)

### Example 92

### Synthesis of BET 92

1.34 g (8.00 mmol) of 3,5-dimethoxybenzylamine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.13 g of the target product.

<¹H-NMR (600 MHz, CDCl₃) of Compound BET 92>

δ: 0.89 (m, 6H), 1.26-1.40 (m, 40H), 1.53 (tt, 2H), 1.66 (m, 2H), 2.02-2.05 (m, 10H), 2.25 (s, 6H), 2.57 (t, 2H), 2.67-2.69 (m, 4H), 3.75 (s, 3H), 3.80-3.83 (m, 9H), 4.46 (s, 2H), 5.34 (m, 4H), 6.16 (s, 1H), 6.36 (m, 1H), 6.65 (m, 2H), 6.87-6.89 (m, 2H), 6.97-6.98 (m, 2H), 7.15-7.18 (m, 2H)

### Example 93

### Synthesis of BET 93

1.34 g (8.00 mmol) of 3,5-dimethoxybenzylamine, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.06 g of the target product.

<¹H-NMR (600 MHz, CDCl₃) of Compound BET 93>

δ: 0.89 (m, 6H), 1.26-1.40 (m, 40H), 1.53 (tt, 2H), 1.66 (m, 2H), 2.02-2.05 (m, 10H), 2.25 (s, 6H), 2.57 (t, 2H), 2.67-2.69 (m, 4H), 3.80 (s, 6H), 4.46 (s, 2H), 5.34 (m, 4H), 6.16 (s, 1H), 6.36 (m, 1H), 6.65 (m, 2H), 6.98 (m, 1H), 7.15-7.18 (m, 3H), 7.24 (m, 1H), 7.50 (m, 1H), 8.54 (m, 1H)

### Example 94

### Synthesis of BET 94

0.98 g (8.00 mmol) of 4-(2-aminoethyl)pyridine, 1.46 g (8.80 mmol) of 3,4-dimethoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 94>

δ: 0.89 (m, 6H), 1.26-1.40 (m, 40H), 1.53 (tt, 2H), 1.66 (m, 2H), 2.02-2.05 (m, 10H), 2.25 (s, 6H), 2.57 (t, 2H), 2.67-2.69 (m, 4H), 3.54-3.59 (m, 4H), 3.75 (s, 3H), 3.83 (s, 3H), 5.34 (m, 4H), 6.16 (s, 1H), 6.87-6.89 (m, 2H), 6.97-6.98 (m, 2H), 7.08 (m, 2H), 7.15-7.18 (m, 2H), 8.44 (m, 2H)

### Example 95

### Synthesis of BET 95

0.98 g (8.00 mmol) of 4-(2-aminoethyl)pyridine, 0.94 g (8.80 mmol) of 4-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 95>

δ: 0.89 (m, 6H), 1.26-1.40 (m, 40H), 1.53 (m, 2H), 1.66 (m, 2H), 2.02-2.05 (m, 10H), 2.25 (s, 6H), 2.57 (t, 2H), 2.67-2.69 (m, 4H), 3.54-3.59 (m, 4H), 5.34 (m, 4H), 6.16 (s, 1H), 6.98 (m, 1H), 7.03-7.08 (m, 4H), 7.15-7.18 (m, 2H), 8.43-8.44 (m, 4H)

### Example 96

### Synthesis of BET 96

1.03 g (8.00 mmol) of 2-(2-aminoethyl)-1-methylpyrrolidine, 1.75 g (8.80 mmol) of tridecanal, and 1.73 g (16.0 mmol) of p-cresol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.06 g of the target product.

<¹H-NMR (600 MHz, CDCl₃) of Compound BET 96>

δ: 0.89 (m, 9H), 1.26-1.66 (m, 70H), 1.82 (m, 2H), 2.02-2.05 (m, 10H), 2.22-2.25 (m, 4H), 2.30-2.40 (m, 5H), 2.57 (t, 2H), 3.18 (t, 2H), 4.78 (s, 1H), 5.34 (m, 4H), 6.94 (m, 1H), 7.13-7.14 (m, 2H)

### Example 97

### Synthesis of BET 97

0.93 g (8.00 mmol) of N,N-diethylethylenediamine, 1.62 g (8.80 mmol) of dodecanal, and 1.73 g (16.0 mmol) of p-cresol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.03 g of the target product.

<¹H-NMR (600 MHz, CDCl₃) of Compound BET 97>

δ: 0.89-0.93 (m, 15H), 1.26-1.40 (m, 58H), 1.53 (tt, 2H), 1.66 (tt, 2H), 1.82 (m, 2H), 2.02-2.05 (m, 10H), 2.31 (s, 3H), 2.46 (q, 4H), 2.57 (t, 2H), 2.62 (t, 2H), 3.30 (t, 2H), 4.78 (s, 1H), 5.34 (m, 4H), 6.94 (m, 1H), 7.13-7.14 (m, 2H)

### Example 98

### Synthesis of BET 98

0.71 g (8.00 mmol) of N,N-dimethylethylenediamine, 0.88 g (8.80 mmol) of 3,3-dimethylbutyraldehyde, and 2.4 g (16.0 mmol) of 4-butylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.95 g of the target product.

<¹H-NMR (600 MHz, CDCl₃) of Compound BET 98>

δ: 0.89-0.91 (m, 18H), 1.26-1.40 (m, 42H), 1.53-1.56 (m, 4H), 1.66-1.70 (m, 4H), 2.02-2.05 (m, 10H), 2.21 (s, 6H), 2.57-2.64 (m, 6H), 3.30 (t, 2H), 4.78 (s, 1H), 5.34 (m, 4H), 6.98 (m, 1H), 7.15-7.18 (m, 2H)

### Example 99

### Synthesis of BET 99

1.14 g (8.00 mmol) of 2-(4-methylpiperazin-1-yl)ethan-1-amine, 0.88 g (8.80 mmol) of 3,3-dimethylbutyraldehyde, and 1.96 g (16.0 mmol) of 2,4-dimethylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 99>

δ: 0.89-0.91 (m, 15H), 1.26-1.40 (m, 40H), 1.53 (m, 2H), 1.66-1.70 (m, 4H), 2.02-2.05 (m, 10H), 2.14-2.15 (m, 6H), 2.27-2.29 (m, 11H), 2.57-2.62 (m, 4H), 3.30 (t, 2H), 4.78 (s, 1H), 5.34 (m, 4H), 6.79 (m, 1H), 6.84 (m, 1H)

### Example 100

### Synthesis of BET 100

1.14 g (8.00 mmol) of N,N-dimethyl-1,4-cyclohexanediamine, 0.94 g (8.80 mmol) of 4-pyridinecarboxaldehyde, and 2.44 g (16.0 mmol) of 4-(2-methoxyethyl) phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 100>

δ: 0.89 (m, 6H), 1.26-1.74 (m, 52H), 2.02-2.05 (m, 10H), 2.22 (s, 6H), 2.57 (m, 3H), 2.85 (t, 2H), 3.40 (s, 3H), 3.52-3.54 (m, 3H), 5.34 (m, 4H), 6.16 (m, 1H), 6.98 (m, 1H), 7.03 (m, 2H), 7.15-7.18 (m, 2H), 8.43 (m, 2H)

### Example 101

### Synthesis of BET 101

1.52 g (8.00 mmol) of 1-benzyl-3-(methylamino)pyrrolidine, 0.76 g (8.80 mmol) of pivalaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 101>

δ: 0.89 (t, 6H), 0.95 (s, 9H), 1.26-1.40 (m, 40H), 1.53-1.77 (m, 6H), 2.02 (m, 8H), 2.22 (s, 3H), 2.29-2.57 (m, 8H), 2.72 (m, 1H), 3.61-3.62 (m, 3H), 3.83 (s, 3H), 5.20 (s, 2H), 5.34 (m, 4H), 6.74 (d, 1H), 6.94 (d, 1H), 7.21-7.29 (m, 5H)

### Example 102

### Synthesis of BET 102

1.14 g (8.00 mmol) of 3-(diethylamino)pyrrolidine, 0.74 g (8.80 mmol) of 3-methyl-2-butenal, and 1.99 g (16.0 mmol) of 3-methoxycatechol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 102>

δ: 0.89 (t, 6H), 1.03 (d, 6H), 1.26-1.45 (m, 40H), 1.55-1.80 (m, 6H), 1.70 (s, 3H), 1.82 (s, 3H), 2.02 (m, 8H), 2.25-2.65 (m, 12H), 2.72 (m, 1H), 3.83 (s, 3H), 4.59 (d, 1H), 5.34 (m, 4H), 5.80 (m, 1H), 6.82 (d, 1H), 7.20 (d, 1H)

### Example 103

### Synthesis of BET 103

1.14 g (8.00 mmol) of N,N-dimethyl-1,4-cyclohexanediamine, 0.76 g (8.80 mmol) of valeraldehyde, and 1.96 g (16.0 mmol) of 3,4-dihydro-5-hydroxy-1H-quinolin-2-one were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 103>

δ: 0.89 (t, 9H), 1.25-1.82 (m, 58H), 2.02-2.05 (m, 10H), 2.22 (s, 6H), 2.43 (m, 2H), 2.57 (m, 3H), 2.88 (m, 2H), 3.54 (m, 1H), 4.78 (m, 1H), 5.34 (m, 4H), 7.23-7.25 (m, 2H), 10.10 (s, 1H)

### Example 104

### Synthesis of BET 104

0.8 g (8.00 mmol) of 1-methylpiperazine, 1.86 g (8.80 mmol) of 5-(4-hydroxypiperidin-1-yl)thiophene-2-carboxaldehyde, and 2.18 g (16.0 mmol) of 4-Isopropylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 104>

δ: 0.89 (t, 6H), 1.20-1.90 (m, 54H), 2.02 (m, 8H), 2.14 (s, 3H), 2.32 (t, 2H), 2.57 (t, 2H), 2.71 (m, 8H), 2.84-2.94 (m, 5H), 5.19-5.34 (m, 6H), 5.81 (d, 1H), 6.44 (d, 1H), 7.10-7.36 (m, 3H)

### Example 105

### Synthesis of BET 105

0.8 g (8.00 mmol) of 1-methylpiperazine, 1.46 g (8.80 mmol) of 2-(2-hydroxyethoxy)benzaldehyde, and 1.95 g (16.0 mmol) of 4-ethylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 105>

δ: 0.89 (t, 6H), 1.18-1.66 (m, 47H), 2.02 (m, 8H), 2.14 (s, 3H), 2.32 (t, 2H), 2.57 (t, 2H), 2.71-2.72 (m, 10H), 4.42-4.45 (m, 4H), 5.14 (m, 1H), 5.34 (m, 4H), 6.92-7.18 (m, 6H), 7.44 (d, 1H)

### Example 106

### Synthesis of BET 106

1.37 g (8.00 mmol) of 1-aminoundecane, 0.94 g (8.80 mmol) of 2-pyridinecarboxaldehyde, and 2.42 g (16.0 mmol) of 2-dimethylaminomethylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 106>

δ: 0.89 (t, 9H), 1.26-1.66 (m, 62H), 2.02-2.05 (m, 10H), 2.21 (s, 6H), 2.57 (m, 2H), 3.18 (m, 2H), 3.57 (s, 2H), 5.34 (m, 4H), 6.16 (m, 1H), 7.16-7.54 (m, 6H), 8.54 (d, 1H)

### Example 107

### Synthesis of BET 107

1.36 g (8.00 mmol) of 2-(tert-butylthio)ethylamine hydrochloride, 0.92 g (8.80 mmol) of 3-(methylthio) propionaldehyde, and 2.42 g (16.0 mmol) of 2-dimethylaminomethylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 107>

δ: 0.89 (t, 6H), 1.26-1.66 (m, 53H), 2.02-2.07 (m, 13H), 2.21 (m, 8H), 2.57-2.70 (m, 6H), 3.57 (m, 4H), 4.78 (m, 1H), 5.34 (m, 4H), 7.22-7.54 (m, 3H)

### Example 108

### Synthesis of BET 108

0.7 g (8.00 mmol) of 2-methylbutylamine, 0.76 g (8.80 mmol) of 2-methylbutyraldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.95 g of the target product.

<¹H-NMR (600 MHz, CDCl₃) of Compound BET 108>

δ: 0.86-0.93 (m, 18H), 1.26-1.66 (m, 48H), 2.02-2.12 (m, 11H), 2.25 (s, 6H), 2.50-2.69 (m, 7H), 3.00-3.25 (m, 2H), 4.68 (m, 1H), 5.34 (m, 4H), 6.98-7.18 (m, 3H)

### Example 109

### Synthesis of BET 109

1.21 g (8.00 mmol) of 1-adamantanamine, 0.92 g (8.80 mmol) of 3-(methylthio) propionaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 109>

δ: 0.89 (t, 6H), 1.26-1.98 (m, 56H), 2.02-2.05 (m, 10H), 2.07 (s, 3H), 2.14 (m, 3H), 2.21 (m, 2H), 2.25 (s, 6H), 2.57-2.69 (m, 8H), 4.78 (m, 1H), 5.34 (m, 4H), 6.98-7.18 (m, 3H)

### Example 110

### Synthesis of BET 110

1.21 g (8.00 mmol) of 1-adamantanamine, 1.12 g (8.80 mmol) of 4-methylthiazole-5-carboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 110>

δ: 0.89 (t, 6H), 1.26-1.98 (m, 56H), 2.02-2.05 (m, 10H), 2.14 (m, 3H), 2.25 (s, 6H), 2.39 (s, 3H), 2.57 (t, 2H), 2.67-2.69 (m, 4H), 5.34 (m, 4H), 6.16 (m, 1H), 6.98-7.18 (m, 3H), 8.74 (s, 1H)

### Example 111

### Synthesis of BET 111

1.21 g (8.00 mmol) of 1-adamantanamine, 1.52 g (8.80 mmol) of 4-(1H-1,2,4-Triazol-1-yl)benzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.12 g of the target product.

<¹H-NMR (600 MHz, CDCl₃) of Compound BET 111>

δ: 0.89 (t, 6H), 1.26-1.98 (m, 56H), 2.02-2.14 (m, 13H), 2.25 (s, 6H), 2.57-2.69 (m, 6H), 5.34 (m, 4H), 6.16 (m, 1H), 6.98-7.64 (m, 7H), 8.49 (s, 1H), 9.24 (s, 1H)

### Example 112

### Synthesis of BET 112

1.14 g (8.00 mmol) of 2-(4-methylpiperazin-1-yl)ethan-1-amine, 1.52 g (8.80 mmol) of 4-(1H-1,2,4-Triazol-1-yl)benzaldehyde, and 2.42 g (16.0 mmol) of 2′-hydroxyacetanilide were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.1 g of the target product.

<¹H-NMR (600 MHz, CDCl₃) of Compound BET 112>

δ: 0.89 (t, 6H), 1.26-1.66 (m, 44H), 2.02-2.05 (m, 10H), 2.10 (s, 3H), 2.14 (s, 3H), 2.29 (m, 8H), 2.57-2.62 (m, 4H), 3.30 (m, 2H), 5.34 (m, 4H), 6.16 (m, 1H), 6.98-7.82 (m, 7H), 8.49 (s, 1H), 9.24 (s, 1H), 10.10 (s, 1H)

### Example 113

### Synthesis of BET 113

1.14 g (8.00 mmol) of 2-(4-methylpiperazin-1-yl)ethan-1-amine, 1.12 g (8.80 mmol) of 4-methylthiazole-5-carboxaldehyde, and 2.42 g (16.0 mmol) of 2′-hydroxyacetanilide were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 113>

δ: 0.89 (t, 6H), 1.26-1.66 (m, 44H), 2.02-2.05 (m, 10H), 2.10 (s, 3H), 2.14 (s, 3H), 2.29 (m, 8H), 2.39 (s, 3H), 2.57-2.62 (m, 4H), 3.30 (m, 2H), 5.34 (m, 4H), 6.16 (m, 1H), 7.28-7.32 (m, 2H), 7.82 (d, 1H), 8.74 (s, 1H), 10.10 (s, 1H)

### Example 114

### Synthesis of BET 114

1.14 g (8.00 mmol) of 2-(4-methylpiperazin-1-yl)ethan-1-amine, 0.93 g (8.80 mmol) of benzaldehyde, and 2.42 g (16.0 mmol) of 2′-hydroxyacetanilide were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 114>

δ: 0.89 (t, 6H), 1.26-1.66 (m, 44H), 2.02-2.05 (m, 10H), 2.10 (s, 3H), 2.14 (s, 3H), 2.29 (m, 8H), 2.57 (t, 2H), 2.62 (t, 2H), 3.30 (t, 2H), 5.34 (m, 4H), 6.16 (m, 1H), 7.10-7.32 (m, 7H), 7.82 (d, 1H), 10.10 (s, 1H)

### Example 115

### Synthesis of BET 115

0.82 g (8.00 mmol) of N-acetylethylenediamine, 0.93 g (8.80 mmol) of benzldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 115>

δ: 0.89 (t, 6H), 1.26-1.66 (m, 44H), 1.78 (s, 3H), 2.02-2.05 (m, 10H), 2.25 (s, 6H), 2.57 (t, 2H), 2.67-2.69 (m, 4H), 3.18 (t, 2H), 3.42 (t, 2H), 5.34 (m, 4H), 6.16 (m, 1H), 6.98-7.29 (m, 8H), 7.50 (s, 1H)

### Example 116

### Synthesis of BET 116

1.03 g (8.00 mmol) of 2-(2-aminoethyl)-1-methylpyrrolidine, 1.62 g (8.80 mmol) of dodecanal, and 1.73 g (16.0 mmol) of p-cresol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 116>

δ: 0.89 (t, 9H), 1.25-1.82 (m, 70H), 2.02-2.05 (m, 10H), 2.22 (s, 3H), 2.25 (m, 1H), 2.31 (s, 3H), 2.35 (m, 2H), 2.57 (t, 2H), 3.18 (t, 2H), 4.78 (m, 1H), 5.34 (m, 4H), 6.94-7.14 (m, 3H)

### Example 117

### Synthesis of BET 117

0.8 g (8.00 mmol) of 1-methylpiperazine, 1.46 g (8.80 mmol) of 2-(2-hydroxyethoxy)benzaldehyde, and 2.44 g (16.0 mmol) of 4-(2-methoxyethyl) phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 117>

δ: 0.89 (t, 6H), 1.26-1.66 (m, 44H), 2.02 (m, 8H), 2.14 (s, 3H), 2.32 (t, 2H), 2.57 (t, 2H), 2.71 (m, 8H), 2.85 (t, 2H), 3.40 (s, 3H), 3.52 (t, 2H), 4.42-4.45 (m, 4H), 5.14 (m, 1H), 5.34 (m, 4H), 6.92-7.44 (m, 7H)

### Example 118

### Synthesis of BET 118

1.03 g (8.00 mmol) of 2-(2-aminoethyl)-1-methylpyrrolidine, 1.12 g (8.80 mmol) of 4-methylthiazole-5-carboxaldehyde, and 1.73 g (16.0 mmol) of p-cresol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.98 g of the target product.

<¹H-NMR (600 MHz, CDCl₃) of Compound BET 118>

δ: 0.89 (t, 6H), 1.26-1.66 (m, 50H), 2.02-2.05 (m, 10H), 2.22 (s, 3H), 2.25 (m, 1H), 2.31 (s, 3H), 2.35 (m, 2H), 2.39 (s, 3H), 2.57 (t, 2H), 3.18 (t, 2H), 5.34 (m, 4H), 6.16 (m, 1H), 6.94-7.14 (m, 3H), 8.74 (s, 1H)

### Example 119

### Synthesis of BET 119

1.36 g (8.00 mmol) of 2-(tert-butylthio)ethylamine hydrochloride, 0.92 g (8.80 mmol) of 3-(methylthio) propionaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.02 g of the target product.

<¹H-NMR (600 MHz, CDCl₃) of Compound BET 119>

δ: 0.89 (t, 6H), 1.26-1.66 (m, 53H), 2.02-2.05 (m, 10H), 2.07 (s, 3H), 2.21 (m, 2H), 2.25 (s, 6H), 2.57-2.70 (m, 10H), 3.57 (t, 2H), 4.78 (m, 1H), 5.34 (m, 4H), 6.98-7.18 (m, 3H)

### Example 120

### Synthesis of BET 120

1.37 g (8.00 mmol) of 1-aminoundecane, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.07 g of the target product.

<¹H-NMR (600 MHz, CDCl₃) of Compound BET 120>

δ: 0.89 (t, 9H), 1.26-1.66 (m, 62H), 2.02-2.05 (m, 10H), 2.25 (s, 6H), 2.57 (t, 2H), 2.67-2.69 (m, 4H), 3.18 (t, 2H), 5.34 (m, 4H), 6.16 (m, 1H), 6.98-7.24 (m, 5H), 7.50 (m, 1H), 8.54 (d, 1H)

### Example 121

### Synthesis of BET 121

1.21 g (8.00 mmol) of 2-amino-1-pyrroline hydrochloride, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.97 g of the target product.

<¹H-NMR (600 MHz, CDCl₃) of Compound BET 121>

δ: 0.89 (t, 6H), 1.26-1.97 (m, 46H), 2.02-2.16 (m, 12H), 2.25 (s, 6H), 2.57 (t, 2H), 2.67-2.69 (m, 4H), 3.50 (m, 1H), 5.34 (m, 4H), 6.16 (m, 1H), 6.98-7.50 (m, 7H), 8.54 (d, 1H)

### Example 122

### Synthesis of BET 122

0.91 g (8.00 mmol) of 1-(2-aminoethyl) pyrrolidine, 0.94 g (8.80 mmol) of 2-pyridinecarboxaldehyde, and 3.08 g (16.0 mmol) of 2,4-dimethoxyphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 122>

δ: 0.89 (t, 6H), 1.26-1.68 (m, 48H), 2.02-2.05 (m, 10H), 2.51 (m, 4H), 2.57 (t, 2H), 2.62 (t, 2H), 3.30 (t, 2H), 3.80 (s, 3H), 3.83 (s, 3H), 5.34 (m, 4H), 6.16 (m, 1H), 6.54-6.65 (m, 2H), 7.16-7.50 (m, 3H), 8.54 (d, 1H)

### Example 123

### Synthesis of BET 123

0.81 g (8.00 mmol) of hexylamine, 0.94 g (8.80 mmol) of 4-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.99 g of the target product.

<¹H-NMR (600 MHz, CDCl₃) of Compound BET 123>

δ: 0.89 (t, 9H), 1.25-1.66 (m, 52H), 2.02-2.05 (m, 10H), 2.25 (s, 6H), 2.57 (t, 2H), 2.67-2.69 (m, 4H), 3.18 (t, 2H), 5.34 (m, 4H), 6.16 (m, 1H), 6.98-7.18 (m, 5H), 8.43 (d, 2H)

### Example 124

### Synthesis of BET 124

0.91 g (8.00 mmol) of 3-methylcyclohexylamine (cis- and trans-mixture), 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.0 g of the target product.

<¹H-NMR (600 MHz, CDCl₃) of Compound BET 124>

δ: 0.86-0.89 (m, 9H), 1.25-1.74 (m, 53H), 2.02-2.05 (m, 10H), 2.25 (s, 6H), 2.57 (t, 2H), 2.67-2.69 (m, 4H), 3.54 (m, 1H), 5.34 (m, 4H), 6.16 (m, 1H), 6.98-7.24 (m, 5H), 7.50 (m, 1H), 8.54 (d, 1H)

### Example 125

### Synthesis of BET 125

0.92 g (8.00 mmol) of 4-heptylamine, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 125>

δ: 0.86-0.92 (m, 15H), 1.26-1.66 (m, 49H), 2.02-2.05 (m, 10H), 2.25 (s, 6H), 2.57 (t, 2H), 2.67-2.69 (m, 4H), 3.40 (m, 1H), 5.34 (m, 4H), 6.16 (m, 1H), 6.98-7.50 (m, 6H), 8.54 (d, 1H)

### Example 126

### Synthesis of BET 126

1.21 g (8.00 mmol) of 2-(4-methoxyphenyl)ethylamine, 0.94 g (8.80 mmol) of 2-pyridinecarboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 126>

δ: 0.89-0.92 (m, 12H), 1.26-1.66 (m, 48H), 2.02-2.05 (m, 10H), 2.25 (s, 6H), 2.57 (t, 2H), 2.67-2.69 (m, 4H), 3.40 (m, 1H), 5.34 (m, 4H), 6.16 (m, 1H), 6.98-7.24 (m, 5H), 7.50 (m, 1H), 8.54 (d, 1H)

### Example 127

### Synthesis of BET 127

1.99 g (8.00 mmol) of 3-aminoquinuclidine dihydrochloride, 1.46 g (8.80 mmol) of 3,4-dimethoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 127>

δ: 0.89 (t, 6H), 1.26-1.66 (m, 48H), 2.02-2.05 (m, 11H), 2.21-2.69 (m, 8H), 3.50 (m, 1H), 3.75 (s, 3H), 3.83 (s, 3H), 5.34 (m, 4H), 6.16 (s, 1H), 6.87-6.97 (m, 3H), 7.10 (d, 1H), 7.30-7.44 (m, 3H)

### Example 128

### Synthesis of BET 128

1.99 g (8.00 mmol) of 3-aminoquinuclidine dihydrochloride, 0.94 g (8.80 mmol) of 2-pyridinecarboxaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 0.94 g of the target product.

<¹H-NMR (600 MHz, CDCl₃) of Compound BET 128>

δ: 0.89 (t, 6H), 1.26-1.66 (m, 48H), 2.02-2.05 (m, 11H), 2.21-2.69 (m, 8H), 3.50 (m, 1H), 5.34 (m, 4H), 6.16 (m, 1H), 6.86-7.50 (m, 7H), 8.54 (d, 1H)

### Example 129

### Synthesis of BET 129

1.99 g (8.00 mmol) of 3-aminoquinuclidine dihydrochloride, 0.94 g (8.80 mmol) of 2-pyridinecarboxaldehyde, and 3.08 g (16.0 mmol) of 2,4-dimethoxyphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.01 g of the target product.

<¹H-NMR (600 MHz, CDCl₃) of Compound BET 129>

δ: 0.89 (t, 6H), 1.26-1.66 (m, 48H), 2.02-2.05 (m, 11H), 2.21-2.69 (m, 8H), 3.50 (m, 1H), 3.75 (s, 3H), 3.83 (s, 3H), 5.34 (m, 4H), 6.16 (m, 1H), 7.10-7.50 (m, 5H), 8.54 (d, 1H)

### Example 130

### Synthesis of BET 130

1.23 g (8.00 mmol) of 4-(1-pyrrolidinyl) piperidine, 1.28 g (8.80 mmol) of indole-6-carboxaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.1 g of the target product.

<¹H-NMR (600 MHz, CDCl₃) of Compound BET 130>

δ: 0.89 (t, 6H), 1.26-1.66 (m, 52H), 2.02-2.05 (m, 8H), 2.32 (t, 2H), 2.41-2.63 (m, 11H), 3.66 (s, 3H), 5.14 (m, 1H), 5.20 (m, 2H), 5.34 (m, 4H), 6.91-7.29 (m, 8H)

### Example 131

### Synthesis of BET 131

1.23 g (8.00 mmol) of 4-(1-pyrrolidinyl) piperidine, 1.45 g (8.80 mmol) of 3,4-ethylenedioxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.12 g of the target product.

<¹H-NMR (600 MHz, CDCl₃) of Compound BET 131>

δ: 0.89 (t, 6H), 1.26-1.66 (m, 52H), 2.02-2.05 (m, 8H), 2.32 (t, 2H), 2.41-2.63 (m, 11H), 3.66 (s, 3H), 4.28 (m, 4H), 5.14 (m, 1H), 5.20 (m, 2H), 5.34 (m, 4H), 6.54-6.97 (m, 5H)

### Example 132

### Synthesis of BET 132

1.23 g (8.00 mmol) of 4-(1-pyrrolidinyl) piperidine, 1.77 g (8.80 mmol) of 9-julolidinecarboxaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.16 g of the target product.

<¹H-NMR (600 MHz, CDCl₃) of Compound BET 132>

δ: 0.89 (t, 6H), 1.26-1.66 (m, 50H), 2.02-2.05 (m, 12H), 2.32 (t, 2H), 2.41-2.63 (m, 11H), 2.79 (t, 4H), 2.96 (t, 2H), 3.37 (t, 4H), 3.83 (s, 3H), 5.14 (s, 1H), 5.20 (s, 2H), 5.34 (m, 4H), 6.54-6.97 (m, 4H)

### Example 133

### Synthesis of BET 133

1.35 g (8.00 mmol) of 4-piperidinopiperidine, 1.28 g (8.80 mmol) of indole-6-carboxaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.11 g of the target product.

<¹H-NMR (600 MHz, CDCl₃) of Compound BET 133>

δ: 0.89 (t, 6H), 1.26-1.66 (m, 54H), 2.02-2.05 (m, 8H), 2.32 (t, 2H), 2.41-2.63 (m, 11H), 3.66 (s, 3H), 5.14 (m, 1H), 5.20 (m, 2H), 5.34 (m, 4H), 6.91-7.29 (m, 8H)

### Example 134

### Synthesis of BET 134

1.35 g (8.00 mmol) of 4-piperidinopiperidine, 1.45 g (8.80 mmol) of 3,4-ethylenedioxybenzaldehyde, and 2.47 g (16.0 mmol) of vanillyl alcohol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.13 g of the target product.

<¹H-NMR (600 MHz, CDCl₃) of Compound BET 134>

δ: 0.89 (t, 6H), 1.26-1.66 (m, 52H), 2.02-2.05 (m, 8H), 2.32 (t, 2H), 2.41-2.63 (m, 11H), 3.66 (s, 3H), 4.28 (t, 3H), 4.27 (t, 3H) 5.14 (m, 1H), 5.20 (m, 2H), 5.34 (m, 4H), 6.91-7.29 (m, 5H)

### Example 135

### Synthesis of BET 135

1.31 g (8.00 mmol) of N-(3-aminopropyl)-N-methylaniline, 1.11 g (8.80 mmol) of 5-methylthiophene-2-carboxaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.08 g of the target product.

<¹H-NMR (600 MHz, CDCl₃) of Compound BET 135>

δ: 0.89 (t, 6H), 1.26-1.66 (m, 44H), 1.78 (m, 2H), 2.02-2.05 (m, 10H), 2.25 (s, 6H), 2.40 (s, 3H), 2.57 (t, 2H), 2.67-2.69 (m, 4H), 2.75 (s, 3H), 3.18 (t, 2H), 3.78 (t, 2H), 5.34 (m, 4H), 6.16 (m, 1H), 6.57-6.74 (m, 5H), 6.98-7.24 (m, 5H)

### Example 136

### Synthesis of BET 136

1.99 g (8.00 mmol) of 3-aminoquinuclidine dihydrochloride, 1.43 g (8.80 mmol) of 4-butylbenzaldehyde, and 2.18 g (16.0 mmol) of 2-Propylphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.05 g of the target product.

<¹H-NMR (600 MHz, CDCl₃) of Compound BET 136>

δ: 0.89-0.94 (m, 12H), 1.26-1.66 (m, 54H), 2.02-2.05 (m, 11H), 2.31 (m, 4H), 2.57 (t, 2H), 2.63-2.69 (m, 6H), 3.50 (m, 1H), 5.34 (m, 4H), 6.16 (m, 1H), 7.08-7.51 (m, 7H)

### Example 137

### Synthesis of BET 137

1.23 g (8.00 mmol) of(S)-(+)-1-(2-pyrrolidinylmethyl) pyrrolidine, 1.65 g (8.80 mmol) of 5-(p-tolyl) isoxazole-3-carboxaldehyde, and 2.21 g (16.0 mmol) of 4-(1-hydroxyethyl) phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.11 g of the target product.

<¹H-NMR (600 MHz, CDCl₃) of Compound BET 137>

δ: 0.89 (t, 6H), 1.26-1.66 (m, 52H), 1.68 (d, 3H), 2.02-2.05 (m, 8H), 2.30 (m, 1H), 2.34 (s, 3H), 2.35 (t, 6H), 2.40 (m, 2H), 2.52 (m, 2H), 2.57 (t, 2H), 5.19 (m, 1H), 5.34 (m, 4H), 6.05 (m, 1H), 6.11 (s, 1H), 7.06-7.35 (m, 5H), 8.01 (m, 2H)

### Example 138

### Synthesis of BET 138

1.35 g (8.00 mmol) of 1-cyclohexylpiperazine, 1.68 g (8.80 mmol) of 3-(4-isopropylphenyl) isobutyraldehyde, and 2.21 g (16.0 mmol) of 2-(2-hydroxyethyl) phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.13 g of the target product.

<¹H-NMR (600 MHz, CDCl₃) of Compound BET 138>

δ: 0.89 (t, 6H), 0.98 (d, 3H), 1.16-1.66 (m, 60H), 2.02-2.05 (m, 8H), 2.31-2.32 (m, 5H), 2.57 (m, 3H), 2.71 (m, 8H), 2.86 (m, 1H), 2.96 (t, 2H), 3.71 (m, 1H), 4.41 (t, 2H), 5.34 (m, 4H), 7.05-7.17 (m, 6H), 7.51 (d, 1H)

### Example 139

### Synthesis of BET 139

1.14 g (8.00 mmol) of N,N-dimethyl-1,3-cyclohexanediamine (cis- and trans-mixture), 1.1 g (8.80 mmol) of 3,5-Dimethylisoxazole-4-carboxaldehyde, and 3.08 g (16.0 mmol) of 2,4-dimethoxyphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.04 g of the target product.

<¹H-NMR (600 MHz, CDCl₃) of Compound BET 139>

δ: 0.89 (t, 6H), 1.26-1.84 (m, 52H), 2.02-2.05 (m, 10H), 2.22 (s, 6H), 2.43 (s, 3H), 2.57 (m, 3H), 2.59 (s, 3H), 3.54 (m, 1H), 3.80 (s, 3H), 3.83 (s, 3H), 5.34 (m, 4H), 6.16 (m, 1H), 6.54-6.65 (m, 2H)

### Example 140

### Synthesis of BET 140

1.14 g (8.00 mmol) of N,N-dimethyl-1,3-cyclohexanediamine (cis- and trans-mixture), 1.5 g (8.80 mmol) of 3,4-ethylenedioxythiophene-2-carboxaldehyde, and 3.08 g (16.0 mmol) of 2,4-dimethoxyphenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.09 g of the target product.

<¹H-NMR (600 MHz, CDCl₃) of Compound BET 140>

δ: 0.89 (t, 6H), 1.26-1.84 (m, 52H), 2.02-2.05 (m, 10H), 2.22 (s, 6H), 2.57 (m, 3H), 3.54 (m, 1H), 3.80 (s, 3H), 3.83 (s, 3H), 4.28 (m, 4H), 5.34 (m, 4H), 6.05 (s, 1H), 6.16 (m, 1H), 6.54-6.65 (m, 2H)

### Example 141

### Synthesis of BET 141

1.43 g (8.00 mmol) of N-benzyl-N,N′-dimethylethylenediamine, 1.46 g (8.80 mmol) of 3-ethoxysalicylaldehyde, and 2.42 g (16.0 mmol) of 4-hydroxyphenylacetamide were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.13 g of the target product.

<¹H-NMR (600 MHz, CDCl₃) of Compound BET 141>

δ: 0.89 (t, 6H), 1.26-1.66 (m, 47H), 2.02-2.05 (m, 10H), 2.22 (s, 3H), 2.57 (t, 2H), 2.62 (t, 2H), 3.30 (t, 2H), 3.66 (s, 2H), 3.68 (s, 3H), 3.85 (s, 2H), 4.13 (t, 2H), 5.34 (m, 4H), 5.42 (s, 2H), 6.16 (m, 1H), 6.91-7.35 (m, 11H)

### Example 142

### Synthesis of BET 142

1.43 g (8.00 mmol) of N-benzyl-N,N′-dimethylethylenediamine, 1.25 g (8.80 mmol) of 3,5,5-trimethylhexanal, and 3.08 g (16.0 mmol) of 2-(2-hydroxycyclohexyl)phenol (cis- and trans-mixture, predominantly cis-isomer) were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified by adsorption and dehydrated by adding silica gel and sodium sulfate, followed by filtering, and the filtrate was concentrated with an evaporator.

The concentrate was dissolved in 21.5 mL of chloroform, and then 2.71 g (9.60 mmol) of oleic acid, 59.0 mg (0.480 mmol) of DMAP, and 2.76 g (1.44 mmol) of EDC hydrochloride were added and reacted at room temperature for 2 hours. The solution after the reaction was purified with a silica gel column using methanol/chloroform to provide 1.15 g of the target product.

<¹H-NMR (600 MHz, CDCl₃) of Compound BET 142>

δ: 0.86-0.91 (m, 18H), 1.07 (m, 2H), 1.26-1.85 (m, 55H), 2.02-2.05 (m, 8H), 2.22 (s, 6H), 2.31-2.35 (m, 2H), 2.46 (m, 4H), 2.57 (t, 2H), 3.31 (m, 1H), 3.66 (s, 2H), 3.81 (m, 1H), 4.99 (m, 1H), 5.34 (m, 4H), 6.91-7.51 (m, 8H)

### Comparative Example 1

### Synthesis of Comparative Example 1

0.91 g (8.00 mmol) of 1-(2-aminoethyl) pyrrolidine, 0.76 g (8.80 mmol) of valeraldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified and dehydrated by adding silica gel and sodium sulfate, followed by filtering, and the filtrate was concentrated with an evaporator.

The concentrate was purified with a silica gel column using methanol/chloroform to provide 0.44 g of the target product.

<¹H-NMR (600 MHz, CDCl₃) of Comparative Example 1>

δ: 0.86-0.88 (t, 3H), 1.20-1.24 (m, 1H), 1.29-1.33 (m, 3H), 1.73-1.78 (m, 6H), 2.45-2.52 (m, 5H), 2.62-2.66 (m, 1H), 2.69-2.75 (m, 2H), 3.65-3.67 (t, 1H), 6.73-6.79 (m, 2H), 6.90-6.91 (dd, 1H), 7.11-7.14 (m, 1H)

### Comparative Example 2

### Synthesis of Comparative Example 2

0.79 g (8.00 mmol) of cyclohexylamine, 1.2 g (8.80 mmol) of 4-methoxybenzaldehyde, and 1.98 g (16.0 mmol) of hordenine were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified and dehydrated by adding silica gel and sodium sulfate, followed by filtering, and the filtrate was concentrated with an evaporator.

The concentrate was purified with a silica gel column using methanol/chloroform to provide 0.61 g of the target product.

<¹H-NMR (600 MHz, CDCl₃) of Comparative Example 2>

δ: 1.14-1.24 (m, 6H), 1.44-1.59 (m, 4H), 2.25 (s, 6H), 2.57 (m, 1H), 2.67-2.69 (m, 4H), 3.78-3.81 (s, 3H), 5.03 (s, 1H), 6.62 (s, 1H), 6.77-6.78 (m, 2H), 6.84-6.86 (m, 2H), 6.93-6.97 (m, 2H)

### Comparative Example 3

### Synthesis of Comparative Example 3

1.14 g (8.00 mmol) of 2-(4-methylpiperazin-1-yl)ethan-1-amine, 1.2 g (8.80 mmol) of 4-methoxybenzaldehyde, and 1.51 g (16.0 mmol) of phenol were mixed and reacted at 80° C. for 3 days. After completing the reaction, ethyl acetate and 1 M hydrochloric acid were added thereto, followed by allowing to stand, and the operation of extracting the aqueous layer, and then again adding ethyl acetate thereto was repeated three times. Subsequently, the aqueous layer was neutralized by adding a 10 M sodium hydroxide aqueous solution, and ethyl acetate was added thereto to extract the target product to the organic layer. The extract was purified and dehydrated by adding silica gel and sodium sulfate, followed by filtering, and the filtrate was concentrated with an evaporator.

The concentrate was purified with a silica gel column using methanol/chloroform to provide 0.55 g of the target product.

<¹H-NMR (600 MHz, CDCl₃) of Comparative Example 3>

δ: 2.27 (s, 3H), 2.28-2.61 (m, 10H), 2.73-2.75 (t, 2H), 3.80 (s, 3H), 4.86 (s, 1H), 6.69-6.72 (m, 1H), 6.76-6.77 (dd, 1H), 6.86-6.88 (m, 3H), 7.13-7.16 (m, 1H), 7.24-7.26 (m, 2H)

### Test Example 1

### Measurement of Liposomal pKa

For evaluation of liposomal pKa, lipid nanoparticles (LNP) containing no nucleic acid were used.

### 1. Preparation of LNP by Microfluidic Method

### (1) Preparation of Ethanol Solution of Lipid

In an Eppendorf tube, a 5 mM ethanol solution of the ionizable lipid of Examples and Comparative Examples, a 3 mM ethanol solution of DOPC, a 10 mM ethanol solution of Chol, and a 0.5 mM ethanol solution of DMG-PEG2k were mixed at the target ratio (ionizable lipid/DOPC/Chol/DMG-PEG2k=52.5/7.5/40/1.5 (molar ratio)) to make a total lipid amount of 800 nmol. Ethanol was then added thereto to prepare an ethanol solution of the lipid (total volume: 1,000 μL).

### (2) Preparation of LNP Using Microfluidic Channel

1,080 μL of an acidic citrate buffer (20 mM, pH 5.0) containing NaCl at a final concentration of 30 mM, and 360 μL of the ethanol solution of the lipid each were measured into a syringe. With an ultrahigh-speed nanomedicine preparation apparatus, NanoAssemblr (available from Precision NanoSystems Inc.), LNP were prepared and collected into a 15 mL tube under conditions of an addition rate of the acidic buffer solution of 3 mL/min, an addition rate of the ethanol solution of the lipid of 1 mL/min, and a syringe holder temperature of 25° C. After adding 1,000 μL of TBS (pH 7.6) to the 15 mL tube, the resulting mixture was transferred to Amicon Ultra 4, and subjected to ultrafiltration under centrifugation conditions (25° C., 1,000 g, and 6 minutes) to concentrate to approximately 100 μL. The resulting concentrate was subjected to an operation of diluting to 4 mL with TBS, and then again concentrating under centrifugation conditions (25° C., 1000 g, and 6 minutes), twice in total. The resulting concentrate was diluted with TBS to make a lipid concentration of 0.5 mM, thereby providing a dispersion liquid containing LNP.

### 2. Measurement of Liposomal pKa

A citrate buffer solution, a sodium phosphate buffer solution, and a Tris-HCl buffer solution each having a concentration of 20 mM and containing NaCl at a final concentration of 150 mM were prepared for various pH values within a range of pH 3.0 to 10.0. A TNS solution (available from Sigma-Aldrich Corporation) was diluted with ultrapure water to 0.6 mM. To a black 96-well plate, 2 μL of the TNS solution, 12 μL of the dispersion liquid containing LNP prepared in [Test Example 1], Section 1, and 186 μL of the buffer solution adjusted to each pH value were added. The plate was shielded from light and shaken at 400 rpm for 10 minutes. With a plate reader (available from TECAN Group Ltd.), the fluorescence intensity (excitation: 321 nm, emission: 447 nm) was measured. With the maximum fluorescence intensity in each LNP set as 100% and the minimum value as 0%, the relative fluorescence intensity was calculated in terms of percentage. The pH value at which the relative fluorescence intensity was 50% was defined as the liposomal pKa. The ionizable lipids used and the liposomal pKa of the LNP are shown in Table 9.

TABLE 9

| Name | Liposomal pKa |
|---|---|
| BET5 | 6.65 |
| BET6 | 6.31 |
| BET19 | 5.85 |
| BET21 | 5.88 |

### 3. Results

As shown in Table 9, the liposomal pKa of each LNP was within the range of pKa (4.5 to 9.5), which was preferred for the endosomal escape.

### Test Example 2

Preparation of mRNA-Encapsulated Particles and Evaluation of Properties

### 1. Preparation of LNP by Microfluidic Method

### (1) Preparation of Ethanol Solution of Lipid

In an Eppendorf tube, a 5 mM ethanol solution of the ionizable lipid of Examples and Comparative Examples, a 5 mM ethanol solution of DOPC, a 10 mM ethanol solution of Chol, and a 0.5 mM ethanol solution of DMG-PEG2k were mixed at the target ratio (ionizable lipid (Comparative Example 1, Comparative Example 2, and Comparative Example 3)/DOPC/Chol/DMG-PEG2k=48.5/7.5/42.5/1.5 (molar ratio)) to make a total lipid amount of 800 nmol. Ethanol was then added thereto to prepare an ethanol solution of the lipid (total volume: 3,200 μL). For Comparative Example 4, each solution was mixed at a ratio of ionizable lipid:DOPC:Chol:DMG-PEG2k=50:10:39:1.5, and ethanol was then added to prepare an ethanol solution of the lipid (total volume: 3,200 μL).

(2) Preparation of Acidic Buffer Solution of Nucleic Acid 4.0 μg of a mRNA solution having a concentration of 0.6 mg/mL was weighed in a 5 mL tube, and an acidic citrate buffer solution (20 mM, pH 5.0) was added thereto to prepare an acidic buffer solution of the nucleic acid (total volume: 15.6 mL).

### (3) Preparation of LNP Using Microfluidic Channel

The acidic buffer solution of the nucleic acid and the ethanol solution of the lipid each were measured into a syringe. With an ultrahigh-speed nanomedicine preparation apparatus, NanoAssemblr (available from Precision NanoSystems Inc.), LNP were prepared and collected into a 15 mL tube under conditions of an addition rate of the acidic buffer solution of nucleic acid of 3 mL/min, an addition rate of the ethanol solution of the lipid of 1 mL/min, and a syringe holder temperature of 25° C. After adding 3,000 μL of TBS buffer solution (pH 7.6) to the 15 mL tube, the resulting mixture was transferred to Amicon Ultra 4, and subjected to ultrafiltration under centrifugation conditions (25° C., 1,000 g, 6 minutes) to concentrate to approximately 500 μL. The resulting concentrate was subjected to an operation of diluting to 4 mL with TBS, and then again concentrating under centrifugation conditions (25° C., 1,000 g, and 6 minutes), twice in total. The resulting concentrate was diluted with TBS to make a lipid concentration of 2 mM, thereby providing a dispersion liquid containing LNP.

2. Measurement of Particle Diameter, PdI, and Zeta Potential of mRNA-Encapsulated LNP

The particle diameter, the PdI (polydispersity index), and the zeta potential of the mRNA-encapsulated LNP prepared in the method in Section 1 above were measured by the dynamic light scattering method (with Zetasizer Nano, available from Malvern Panalytical Ltd.). The ionizable lipids used and the results are shown in Tables 10-1 to 10-3.

TABLE 10-1

| | Particle | | Zeta | |
|---|---|---|---|---|
| Ionizable | diameter | | potential | Aggregation |
| lipid used | (nm) | PdI | (mV) | (%) |
| BET1 | 79 | 0.08 | −2.9 | 0 |
| BET3 | 103 | 0.06 | 0.5 | 0 |
| BET4 | 86 | 0.02 | −2.3 | 0 |
| BET5 | 101 | 0.07 | 0.0 | 0 |
| BET6 | 83 | 0.04 | −1.6 | 0 |
| BET13 | 81 | 0.08 | −5.3 | 0 |
| BET15 | 79 | 0.06 | −2.6 | 0 |
| BET16 | 84 | 0.06 | −2.7 | 0 |
| BET17 | 86 | 0.07 | −3.9 | 0 |
| BET18 | 83 | 0.09 | −6.6 | 0 |
| BET19 | 84 | 0.07 | −5.3 | 0 |
| BET20 | 87 | 0.04 | −3.5 | 0 |
| BET21 | 88 | 0.03 | −2.9 | 0 |
| BET22 | 84 | 0.05 | −4.6 | 0 |
| BET24 | 114 | 0.01 | 0.3 | 0 |
| BET25 | 79 | 0.07 | −1.8 | 0 |
| BET26 | 88 | 0.05 | −2.4 | 0 |
| BET27 | 97 | 0.06 | 0.3 | 0 |
| BET32 | 96 | 0.07 | −2.7 | 0 |
| BET34 | 115 | 0.03 | 0.4 | 0 |
| BET35 | 104 | 0.05 | −4.9 | 0 |
| BET36 | 78 | 0.07 | −0.9 | 0 |
| BET37 | 88 | 0.06 | −3.8 | 0 |
| BET38 | 85 | 0.05 | −1.3 | 0 |
| BET39 | 82 | 0.07 | −1.0 | 0 |
| BET40 | 89 | 0.07 | −2.2 | 0 |
| BET42 | 98 | 0.03 | 0.7 | 0 |
| BET43 | 84 | 0.06 | −1.0 | 0 |

TABLE 10-2

| | Particle | | Zeta | |
|---|---|---|---|---|
| Ionizable | diameter | | potential | Aggregation |
| lipid used | (nm) | PdI | (mV) | (%) |
| BET45 | 83 | 0.09 | −1.5 | 0 |
| BET47 | 80 | 0.09 | 1.3 | 0 |
| BET48 | 91 | 0.14 | −1.9 | 0 |
| BET49 | 104 | 0.03 | 0.6 | 0 |
| BET50 | 92 | 0.06 | −0.9 | 0 |
| BET51 | 90 | 0.06 | −1.5 | 0 |
| BET54 | 92 | 0.10 | −2 | 0 |
| BET55 | 87 | 0.07 | −1 | 0 |
| BET56 | 122 | 0.12 | 4.8 | 0 |
| BET57 | 80 | 0.06 | 2.5 | 0 |
| BET58 | 87 | 0.06 | −2.3 | 0 |
| BET59 | 94 | 0.07 | −2.3 | 0 |
| BET60 | 99 | 0.04 | 0.3 | 0 |
| BET61 | 95 | 0.05 | −0.3 | 0 |
| BET65 | 91 | 0.04 | −2.7 | 0 |
| Comparative | 104 | 0.34 | −5.8 | 3 |
| Example 1 | | | | |
| Comparative | 105 | 0.20 | −4.2 | 3 |
| Example 2 | | | | |
| Comparative | 97 | 0.22 | −5.7 | 7 |
| Example 3 | | | | |
| Comparative | 88 | 0.15 | −3.8 | 0 |
| Example 4 | | | | |

TABLE 10-3

| | Particle | | Zeta | |
|---|---|---|---|---|
| Ionizable | diameter | | potential | Aggregation |
| lipid used | (nm) | PdI | (mV) | (%) |
| BET75 | 87 | 0.11 | −5.3 | 0 |
| BET76 | 87 | 0.07 | −2.0 | 0 |
| BET77 | 81 | 0.05 | −1.4 | 0 |
| BET79 | 77 | 0.05 | −4.6 | 0 |
| BET80 | 173 | 0.12 | 0.2 | 0 |
| BET81 | 137 | 0.06 | −0.1 | 0 |
| BET82 | 109 | 0.03 | −1.2 | 0 |
| BET88 | 109 | 0.02 | −6.7 | 0 |
| BET90 | 86 | 0.06 | −2.5 | 0 |
| BET93 | 106 | 0.06 | 0.8 | 0 |
| BET97 | 87 | 0.05 | −4.7 | 0 |
| BET98 | 83 | 0.08 | −4.5 | 0 |
| BET99 | 97 | 0.01 | −1.9 | 0 |
| BET100 | 109 | 0.10 | 1.6 | 0 |
| BET105 | 81 | 0.08 | −6.8 | 0 |
| BET117 | 84 | 0.06 | −4.2 | 0 |
| BET120 | 106 | 0.03 | −2.8 | 0 |

### 3. Results

As shown in Tables 10-1 to 10-3, each mRNA-encapsulated LNP had a particle diameter of 30 to 300 nm as a preferred embodiment, and the charge at physiological pH (zeta potential) thereof was also within a preferred range (−15 to +15 mV). On the other hand, Comparative Examples 1, 2, and 3, which were obtained by removing the aromatic ester moiety from the structures described in Examples 16, 32, and 34, respectively, caused aggregation, failing to provide particles satisfactory as lipid nanoparticles. Accordingly, it was demonstrated that in the structures of the present invention, the molecules derived from the amine and the linker structure functioned as an ionizable lipid that formed LNP only by introducing the aromatic ester thereto.

### Test Example 3

Evaluation of Gene Expression in Hela Cells in vitro
1. Preparation of mRNA-Encapsulated LNP

LNP encapsulating mRNA expressing luciferase were prepared by the method described in Test Example 2, Section 1.

### 2. Time-Course Evaluation of Gene Expression in Hela Cells

HeLa cells, i.e., human cervical cancer cells, were seeded in 3.5 cm dishes to make 5.0×10⁴ cells/2 mL/dish, 24 hours before transfection. After 24 hours, the medium was replaced with a culture medium containing 0.1 mM D-luciferin (D-MEM). The prepared mRNA-encapsulated LNP were diluted with PBS or TBS to make a concentration of the mRNA of approximately 1 μg/mL. The diluted mRNA-encapsulated LNP solution (approximately 200 μL, mRNA: 0.2 μg) was added to a 3.5 cm dish and set in an incubator type luminometer, Kronos Dio. The luminescence intensity of luciferase was measured for 2 minutes every one hour. The 24-hour cumulative luciferase luminescence intensity was calculated from the obtained time course of expression. The ionizable lipids used, the 24-hour cumulative luciferase luminescence intensity, and the relative luminescence intensity (=(24-hour cumulative luciferase luminescence intensity using the ionizable lipid of Example)/(24-hour cumulative luciferase luminescence intensity using the ionizable lipid of Comparative Example 4)) are shown in Table 11. In Table 11, “E+0a” (a: integer) represents “10^{a}”. For example, “1.0E+06” represents “1.0×10⁶”.

TABLE 11

| | 24-hour cumulative | |
|---|---|---|
| Ionizable | luciferase luminescence | Relative |
| lipid used | intensity (RLU) | intensity |
| BET38 | 1.0.E+08 | 1.84 |
| BET39 | 1.2.E+08 | 2.11 |
| BET42 | 1.2.E+08 | 2.12 |
| BET47 | 1.2.E+08 | 2.19 |
| BET49 | 2.5.E+08 | 4.33 |
| BET50 | 2.6.E+08 | 4.61 |
| BET51 | 2.6.E+08 | 4.64 |
| BET54 | 2.7.E+08 | 4.70 |
| BET55 | 2.8.E+08 | 4.97 |
| BET58 | 4.3.E+08 | 7.60 |
| Comparative | 5.7.E+07 | 1.00 |
| Example 4 | | |

### 3. Results

A higher 24-hour cumulative luciferase luminescence intensity, i.e., a higher total luciferase activity, means a higher gene expression. The LNP containing the ionizable lipids of the Examples shown in Table 11 exhibited a superior gene expression activity as compared to the LNP containing the ionizable lipid of Comparative Example 4. Accordingly, it was revealed that the LNP containing the ionizable lipids of the present invention were useful as LNP capable of promoting expression of mRNA.

### Test Example 4

### Evaluation of Gene Expression In Vivo

1. Preparation of mRNA-Encapsulated LNP

LNP encapsulating mRNA expressing luciferase (ionizable lipid/DOPC/Chol/DMG-PEG2k=48.5/7.5/42.5/1.5 (molar ratio)) were prepared by the method described in Test Example 3, Section 1.

### 2. Evaluation of Gene Expression In Vivo

The prepared dispersion liquid of mRNA-encapsulated LNP was administered via the tail vein of Balb/c mice (male, 5 weeks old). The dose of the mRNA was set at 0.05 mg per 1 kg of body weight of the mice, and the dose of the dispersion liquid of mRNA-encapsulated LNP was set at 10 μL per 1 g of body weight of the mice. At 4.5 hours after the administration of the dispersion liquid of the mRNA-encapsulated LNP, a PBS solution of potassium D-luciferin was administered into the peritoneal cavity of the mice. The dose of potassium D-luciferin was set at 3 mg per mouse, and the dose of the PBS solution of potassium D-luciferin was set at 200 μL per mouse. At 15 minutes after the administration of the PBS solution of potassium D-luciferin, the mice were euthanized, and the livers were excised. The luminescence in the livers was quantified with an in vivo imaging system (IVIS) at an exposure time of 10 seconds. The amount of luminescence in the livers was quantified by image analysis using Live Imaging Software attached to the IVIS. The amount of luminescence (photons/sec) was calculated from the acquired images, and used as an index of the gene expression activity. The ionizable lipids used and the amount of luminescence in the livers are shown in Table 12. In Table 12, “E+0a” (a: integer) represents “10ª”. For example, “3.94E+09” represents “3.94×10⁹”. As a comparative example, evaluation was also performed in the same manner for mice to which only PBS was administered, and the result is shown in Tables 12 and 12-2.

TABLE 12

| | Amount of luminescence |
|---|---|
| Ionizable lipid | in the livers |
| BET1 | 2.7.E+06 |
| BET5 | 7.6.E+07 |
| BET6 | 5.3.E+07 |
| BET15 | 2.7.E+08 |
| BET16 | 8.6.E+08 |
| BET19 | 3.3.E+08 |
| BET21 | 4.6.E+08 |
| BET24 | 1.9.E+07 |
| BET26 | 2.6.E+09 |
| BET27 | 1.3.E+08 |
| BET32 | 3.1.E+08 |
| BET34 | 8.5.E+06 |
| BET35 | 3.8.E+08 |
| BET36 | 2.0.E+09 |
| BET37 | 2.5.E+08 |
| BET38 | 2.6.E+09 |
| BET39 | 3.4.E+09 |
| BET40 | 3.7.E+09 |
| BET42 | 8.6.E+06 |
| BET43 | 1.5.E+08 |
| BET47 | 1.4.E+08 |
| BET48 | 2.7.E+09 |
| BET49 | 2.9.E+09 |
| BET50 | 7.4.E+09 |
| BET51 | 5.9.E+09 |
| BET54 | 2.2.E+09 |
| BET55 | 3.1.E+09 |
| BET57 | 8.0.E+05 |
| BET58 | 3.6.E+09 |
| PBS | −1.20E+06 |

TABLE 12-2

| | Amount ofluminescence |
|---|---|
| Ionizable lipid | in the livers |
| BET077 | 1.4 E+09 |
| BET078 | 1.6 E+09 |
| BET080 | 6.0 E+07 |
| BET081 | 2.7 E+07 |
| BET082 | 1.8 E+08 |
| BET083 | 6.7 E+08 |
| BET094 | 9.0 E+07 |
| BET098 | 2.0 E+07 |
| BET099 | 1.4 E+06 |
| BET100 | 3.9 E+08 |
| BET101 | 2.2 E+06 |
| BET106 | 2.8 E+07 |

The amounts of luminescence in the livers shown in Tables 12 and 12-2 demonstrated that the ionizable lipids of the Examples were able be used as nucleic acid delivery carriers.

### Test Example 5

Evaluation of Gene Expression in Extrahepatic Organs with Respect to Liver In Vivo
1. Preparation of mRNA-encapsulated LNP

LNP encapsulating mRNA expressing luciferase (ionizable lipid/DOPC/Chol/DMG-PEG2k=48.5/7.5/42.5/1.5 (molar ratio)) were prepared by the method described in Test Example 3, Section 1.

### 2. Evaluation of Gene Expression In Vivo

The prepared dispersion liquid of mRNA-encapsulated LNP was administered via the tail vein of Balb/c mice (male, 5 weeks old). The dose of mRNA was set at 0.05 mg per 1 kg of body weight of the mice, and the dose of the dispersion liquid of mRNA-encapsulated LNP was set at 10 μL per 1 g of body weight of the mice. At 4.5 hours after the administration of the dispersion liquid of mRNA-encapsulated LNP, a PBS solution of potassium D-luciferin was administered into the peritoneal cavity of the mice. The dose of potassium D-luciferin was set at 3 mg per mouse, and the dose of the PBS solution of potassium D-luciferin was set at 200 μL per mouse. At 15 minutes after administration of the PBS solution of potassium D-luciferin, the mice were euthanized and the livers were excised. The luminescence in the livers and also the spleens and kidneys was quantified with an in vivo imaging system (IVIS) at an exposure time of 10 seconds. The amounts of luminescence in the livers, the spleens, and kidneys were quantified by image analysis using Live Imaging Software attached to the IVIS. The amount of luminescence (photons/sec) was calculated from the acquired images and used as an index of the gene expression activity. The ratio of the amounts of luminescence in the spleens and the kidneys with respect to the amount of luminescence in the livers for each ionizable lipid used are shown in Tables 13 and 14.

TABLE 13

| | Ratio of amount of luminescence |
|---|---|
| Ionizable lipid | in spleens to livers (%) |
| BET5 | 1.5 |
| BET24 | 4.9 |
| BET27 | 1.1 |
| BET32 | 1.3 |
| BET47 | 8.7 |
| BET42 | 36.3 |
| BET32 | 1.3 |
| BET34 | 37.4 |
| BET57 | 177.1 |
| Comparative | 0.2 |
| Example 4 | |

TABLE 14

| | Ratio of amount of luminescence |
|---|---|
| Ionizable lipid | in kidneys to livers (%) |
| BET1 | 5.4 |
| BET34 | 1.1 |
| BET42 | 10.8 |
| BET45 | 6.3 |
| BET47 | 0.7 |
| BET57 | 10.1 |
| Comparative | 0.1 |
| Example 4 | |

### 3. Results

Table 13 and 14 show the amount of luminescence in the extrahepatic organs with respect to the amount of luminescence in the liver for each of the ionizable lipids. With the use of the ionizable lipid LNP described in the description herein, the expression in the spleens and the kidneys relative with respect to the liver was confirmed only with the ionizable lipid alone, the PEG lipid, Chol, and the helper lipid, which showed that the ionizable lipid described in the description herein was useful for expression in the spleens and expression in the kidneys, as compared to Comparative Example 4.

Furthermore, the results of Table 13 and Table 14 suggested that the ionizable lipid having a cyclic tertiary amine described in the description herein potentially had a higher delivery efficiency to extrahepatic organs, such as the spleens and kidneys, as compared to the liver.

It was clarified from the above that the amine structure and the linker structure described in the description herein functioned as an ionizable lipid in LNP by introducing the aromatic ester moiety, and the LNP containing the ionizable lipid of the present invention were useful as LNP capable of promoting expression of mRNA.

### INDUSTRIAL APPLICABILITY

The ionizable lipid of the present invention is useful as a nucleic acid medicine, a gene therapy, a biochemistry experiment, and the like.

1. An ionizable lipid used for delivering pharmaceuticals, comprising at least one linear or cylcic tertiary amino group, at least one phenyl group having an ester bond at an o-position, and an aliphatic hydrocarbon group having 4 or more carbon atoms.

2. The ionizable lipid according to claim 1, wherein the ionizable lipid is selected from the group consisting of compounds represented by the following formulae (1) and (2): 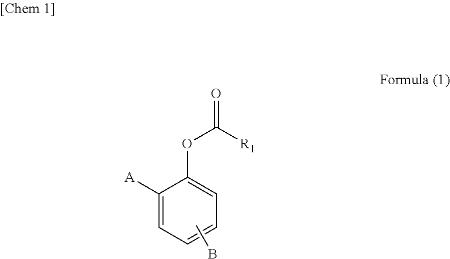
wherein, in the formula (1),
A represents an aliphatic compound having 15 to 45 carbon atoms, having at least one linear or cyclic tertiary amino group, which may have at least any structure selected from the group consisting of a hydroxy group, an ether group, an ester group, a nitrile group, a amide group, a nitro group, a thioamide group, a sulfide group, a urea bond, a thioester bond, a thiocarbamate bond, a thiourea bond, a sulfonamide bond, a sulfonyl group, and a heterocyclic ring having at least any atom selected from nitrogen, oxygen, and sulfur, and may be bonded to an aromatic compound,
B represents (i) a hydrogen atom, (ii) an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur, or (iii) R₁′—CO—O—R₁₄—*, wherein * represents a bonding position, R₁′ represents an aliphatic hydrocarbon group having 10 to 30 carbon atoms, and R₁₄ represents an aliphatic hydrocarbon group having 0 to 8 carbon atoms,
the group represented by B may be substituted at any one substitution position of four substitution positions of the aromatic ring shown in formula (1), or may be substituted at two or more substitution positions thereof, and in the case where the groups represented by B are substituted at two or more substitution positions, the multiple groups may be the same as or different from each other, and the groups at the adjacent two substitution positions may form a ring, and
R₁ represents an aliphatic hydrocarbon group having 10 to 30 carbon atoms, 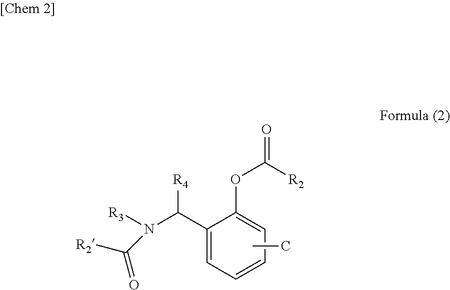
wherein, in the formula (2),
R₂ and R₂′ each independently represent an aliphatic hydrocarbon group having 10 to 30 carbon atoms,
R₃ represents an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound,
R₄ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound, and
C represents R₁₂—R₁₃—*, wherein * represents a bonding position, R₁₂ represents a tertiary amino group having 2 to 6 carbon atoms, and R₁₃ represents an aliphatic compound having 2 to 30 carbon atoms, which may have a hydroxy group, and may have an ester bond.

3. The ionizable lipid according to claim 2, wherein the ionizable lipid is selected from the group consisting of compounds represented by the following formulae (3) to (6): 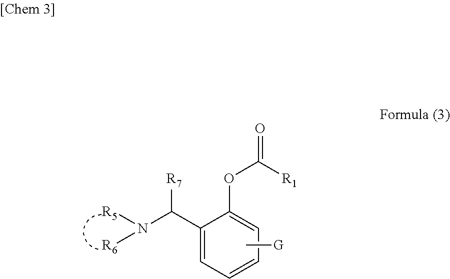
wherein, in the formula (3),
R₁ has the same meaning as in the formula (1),
R₅ and R₆ each independently represent an aliphatic compound having 1 to 30 carbon atoms, 0 to 5 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, and a heterocyclic ring having at least any atom selected from nitrogen, oxygen, and sulfur, and R₅ and R₆ each may be independent from each other, or may be connected to each other to form a ring,
R₇ represents an aliphatic compound having 2 to 30 carbon atoms, 2 to 10 oxygen atoms, 0 to 5 nitrogen atoms, and 0 to 2 sulfur atoms, having an ester bond, which may have at least any structure selected from the group consisting of a hydroxy group, an ether group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound,
G represents a hydrogen atom or an aliphatic compound or an aromatic compound having 1 to 15 carbon atoms, 0 to 10 oxygen atoms, 0 to 5 nitrogen atoms, and 0 to 3 sulfur atoms, which may contain a halogen atom, and may have at least any structure selected from the group consisting of a hydroxy group, an ether group, an ester group, a nitrile group, an amide group, a nitro group, a thioamide group, a sulfide group, a sulfonamide group, a sulfone group, and a heterocyclic ring having at least any atom selected from nitrogen, oxygen, and sulfur, and
the group represented by G may be substituted at any one substitution position of four substitution positions of the aromatic ring shown in formula (3), or may be substituted at two or more substitution positions thereof, and in the case where the groups represented by G are substituted at two or more substitution positions, the multiple groups may be the same as or different from each other, and the groups at the adjacent two substitution positions may form a ring, 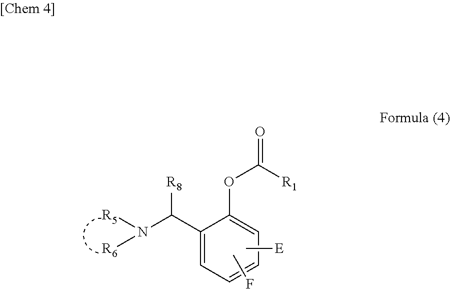
wherein in the formula (4),
R₁ has the same meaning as in the formula (1),
R₅ and R₆ have the same meanings as in the formula (3),
R₈ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound,
E represents a hydrogen atom, a nitro group, a methoxy group, a nitrile group, a methyl group, a tert-butyl group, or a 1,1-dimethylpropyl group, and
F represents R₁′—CO—O—R₁₄—*, wherein * represents a bonding position, R₁′ has the same meaning as in the formula (1), and R₁₄ represents an aliphatic hydrocarbon group having 0 to 8 carbon atoms, 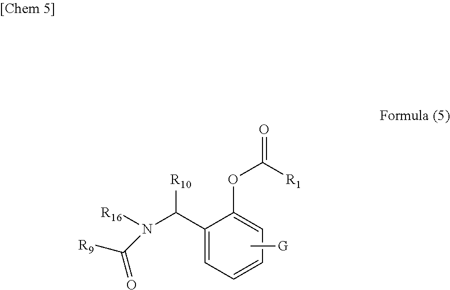
wherein, in the formula (5),
R₁ has the same meaning as in the formula (1),
R₉ represents an aliphatic hydrocarbon group having 10 to 30 carbon atoms,
R₁₀ represents an aliphatic compound having at least one linear or cyclic tertiary amine structure, having 2 to 15 carbon atoms, 0 to 10 oxygen atoms, and 1 to 5 nitrogen atoms, which may have at least any functional group selected from the group consisting of an ester group, an amide group, a carbamate group, an ether group, a urea group, a thioamide group, a sulfide group, and a sulfonamide group, and may be bonded to an aromatic compound,
R₁₆ represents an aliphatic compound having 2 to 15 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a sulfonyl group, a heterocyclic ring having at least any atom selected from nitrogen, oxygen, and sulfur, and an azido group, and may be bonded to an aromatic compound, and
G has the same meaning as in the formula (3), 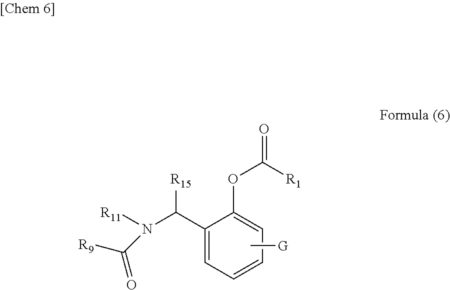
wherein, in the formula (6),
R₁ has the same meaning as in the formula (1),
R₉ has the same meaning as in the formula (5),
R₁₁ represents an aliphatic compound having at least one linear or cyclic tertiary amine structure, having 1 to 15 carbon atoms, which may be bonded to an aromatic compound,
R₁₅ represents an aliphatic compound having 2 to 20 carbon atoms, 0 to 10 oxygen atoms, 0 to 5 nitrogen atoms, and 0 to 2 sulfur atoms, which may have at least any structure selected from the group consisting of a hydroxy group, an ether group, an ester group, a nitrile group, an amide group, a nitro group, a thioamide group, a sulfide group, a sulfonamide group, a heterocyclic ring having at least any atom selected from nitrogen, oxygen, and sulfur, and an azido group, and may be bonded to an aromatic compound, and
G has the same meaning as in the formula (3).

4. The ionizable lipid according to claim 2, wherein the compound represented by the formula (2) satisfies the following requirements:
R₂ and R₂′ each independently represent an alkyl group having 10 to 30 carbon atoms, an alkenyl group having 10 to 30 carbon atoms, or an alkynyl group having 10 to 30 carbon atoms,
R₃ represents an aliphatic compound having 3 to 13 carbon atoms, 0 to 4 oxygen atoms, 0 to 1 nitrogen atom, and 0 to 1 sulfur atom, which may have at least any structure selected from the group consisting of an ether group, an ester group, a nitrile group, an amide group, a nitro group, a sulfide group, a sulfonamide group, a sulfonyl group, and a heterocyclic ring having at least any atom selected from nitrogen, oxygen, and sulfur, and may be bonded to an aromatic compound,
R₄ represents an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound, and
C represents any of structures represented by the following formulae (7) to (9): 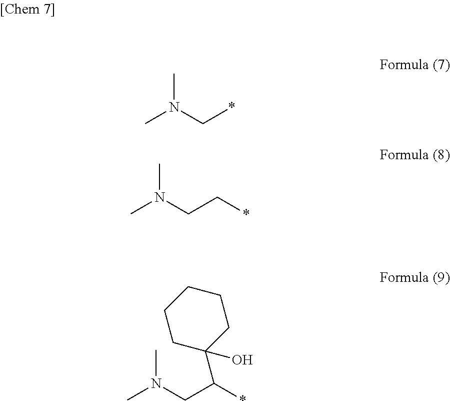
wherein, in the formulae (7) to (9), * represents a bonding position.

5. The ionizable lipid according claim 3, wherein the compounds represented by the formulae (3) to (6) satisfy the following requirements:
R₁ and R₁′ each independently represent an alkyl group having 10 to 30 carbon atoms, an alkenyl group having 10 to 30 carbon atoms, or an alkynyl group having 10 to 30 carbon atoms,
R₅ and R₆ each independently represent an aliphatic compound having 1 to 15 carbon atoms, 0 to 2 oxygen atoms, 0 to 1 nitrogen atom, and 0 to 1 sulfur atom, which may form a ring, may contain an unsaturated bond, and may have at least any structure selected from the group consisting of an ether group, an ester group, a nitrile group, an amide group, a nitro group, a sulfide group, a sulfonamide group, and a heterocyclic ring having at least any atom selected from nitrogen, oxygen, and sulfur, and R₅ and R₆ each may be independent from each other, or may be connected to each other to form a ring,
R₇ represents an aliphatic compound having 5 to 30 carbon atoms, 2 to 4 oxygen atoms, 0 to 1 nitrogen atom, and 0 to 1 sulfur atom, having an ester bond, which may contain an unsaturated bond, may have a branch, may form a ring, and may have at least any structure selected from the group consisting of an aromatic ring, an ether group, and a heterocyclic ring having at least any atom selected from nitrogen, oxygen, and sulfur,
R₈ represents an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound,
R₁₀ represents any of structures represented by the following formulae (10) to (14): 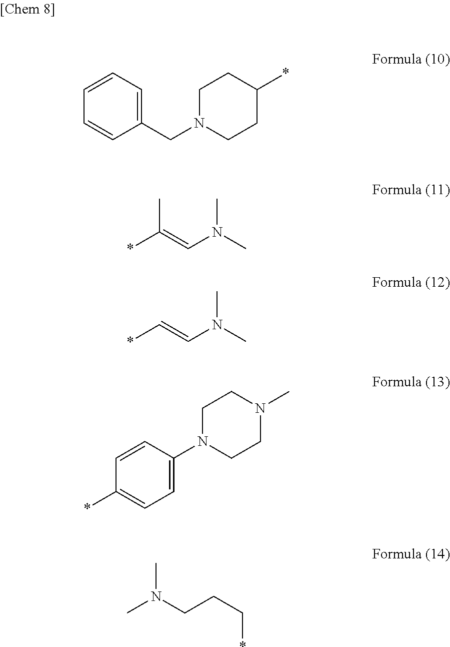
wherein, in the formulae (10) to (14), * represents a bonding position,
R₁₄ represents an alkylene group having 0 to 8 carbon atoms, an alkenediyl group having 0 to 8 carbon atoms, or an alkynediyl group having 0 to 8 carbon atoms,
R₁₅ represents an aliphatic compound having 3 to 13 carbon atoms, 0 to 4 oxygen atoms, 0 to 3 nitrogen atoms, and 0 to 2 sulfur atoms, which may have at least any structure selected from the group consisting of an ether group, an ester group, a nitrile group, an amide group, a nitro group, a sulfide group, and a heterocyclic ring having at least any atom selected from nitrogen, oxygen, and sulfur, and may be bonded to an aromatic compound, and
R₁₆ represents an aliphatic compound having 3 to 13 carbon atoms, 0 to 4 oxygen atoms, 0 to 1 nitrogen atom, and 0 to 1 sulfur atom, which may have at least any structure selected from the group consisting of an ether group, an ester group, a nitrile group, an amide group, a nitro group, a sulfide group, a sulfonamide group, a sulfonyl group, and a heterocyclic ring having at least any atom selected from nitrogen, oxygen, and sulfur, and may be bonded to an aromatic compound.

6. A lipid membrane structure comprising the ionizable lipid according to claim 1 as a constitutional lipid of the membrane.

7. The lipid membrane structure according to claim 6, wherein the lipid membrane structure further comprises a nucleic acid.

8. A nucleic acid transfer agent comprising the ionizable lipid according to claim 1.

9. The nucleic acid transfer agent according to claim 8, wherein the nucleic acid transfer agent further comprises a nucleic acid.

10. A pharmaceutical composition comprising the ionizable lipid according to claim 1.

11. The pharmaceutical composition according to claim 10, wherein the pharmaceutical composition further comprises a nucleic acid.

12. A method for transferring a nucleic acid contained in a nucleic acid transfer agent into cells, comprising bringing the cells into contact with the nucleic acid transfer agent according to claim 8 in vitro.

13. A method for transferring a nucleic acid contained in a nucleic acid transfer agent to target cells of a living body, comprising administrating the nucleic acid transfer agent according to claim 8 into the living body.

14. A method for producing a cellular medicine containing cells that express a gene in a nucleic acid, comprising bringing cells into contact with the nucleic acid transfer agent according to claim 8 to transfer the nucleic acid contained in the nucleic acid transfer agent into the cells.
